(19) United States
(12) Patent Application Publication
(10) Pub. No.: US 2024/0109914 A1
(43) Pub. Date: Apr. 4, 2024

(54) IMIDAZOTHIAZOLE COMPOUNDS AND ANALOGS THEREOF AND METHODS FOR TREATING NEMATODE INFECTIONS IN PLANTS

(71) Applicant: The Governing Council of The University of Toronto, Toronto (CA)

(72) Inventors: Andrew Burns, Toronto (CA); Peter Roy, Toronto (CA); Rachel Baker, Toronto (CA); Mark Lautens, Toronto (CA)

(21) Appl. No.: 18/271,522

(22) PCT Filed: Jan. 13, 2022
(86) PCT No.: PCT/CA2022/050050

§ 371 Date: Jul. 10, 2023

Related U.S. Application Data
(60) Provisional application No. 63/137,010, filed on Jan. 13, 2021.
(60) Provisional application No. 63/137,517, filed on Jan. 14, 2021.

(51) Int. Cl. C07D 513/04; A01N 43/90; A01P 5/00

(52) U.S. Cl. CPC C07D 513/04; A01N 43/90; A01P 5/00

(57) ABSTRACT

The present application relates to the treatment of plant nematode infections. For example, the application relates to the use of one or more compounds of Formula (I) as defined herein, or compositions comprising these compounds, for treatment of a nematode infection or a disease, disorder or condition arising from a nematode infection in a plant in need thereof. Also included are novel compounds within the scope of Formula I and compositions and uses of these compounds for treatment of a nematode infection or a disease, disorder or condition arising from a nematode infection in a plant in need thereof.

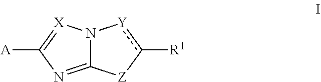

### RELATED APPLICATIONS

The present application claims the benefit of priority from U.S. provisional patent application No. 63/137,010, filed on Jan. 13, 2021, and U.S. provisional patent application No. 63/137,517, filed on Jan. 14, 2021, the contents of each of which are incorporated by reference in their entirety.

### FIELD

The present application relates to the treatment of nematode infections in plants. For example, the application relates to the use of one or more compounds as disclosed herein for treatment of a nematode infection or a disease, disorder or condition arising from a nematode infection in a plant in need thereof.

### INTRODUCTION

Over the next 30 years, the world population is expected to reach 9.8 billion people¹. This level of population growth alone is likely to jeopardize global food security, but as developing nations begin to incorporate more protein, sugar, and animal fats in their diets there will be a corresponding increase in per-capita food consumption as well^{2,3,4}, further compounding the problem. To make matters worse, arable land for agricultural expansion is scarce and efforts at land conversion are inhibited by social and ecological factors³⁻⁷. Intensifying production from currently farmed land will therefore be essential to ensure global food security^{3,7-9}.

Pest organisms and pathogens that infect livestock and damage crops can dramatically reduce the output of farmed land^{4,10,11}. Parasitic nematodes are a particularly destructive agricultural pathogen that infect various commercially valuable animals and plants¹¹⁻¹⁹. Nematode infections of livestock result in global losses to farmers of $10 billion or more annually^{11,16-18}, and plant-parasitic nematodes (PPNs) are responsible for well over $100 billion in crop losses each year²⁰⁻²². PPNs can be especially damaging—lowering crop yields by more than 80% in some circumstances¹⁹. In particular, the plant-parasitic root-knot nematode Meloidogyne incognita, owing to its vast global distribution and broad host range, is arguably the most damaging plant pathogen in the world^{12,14,23,24}.

Although sound agricultural practices, biologicals, and GMOs can reduce PPN infestation, synthetic small-molecule nematicides have been an essential part of a complete crop protection strategy for decades^{21,22,25-27}. In recent years, however, many effective synthetic nematicides have been restricted or banned due to adverse effects on human health and the environment²⁸⁻³¹. For example, the ozone-depleting fumigant methyl bromide, as well as many of the neurotoxic organophosphate and carbamate nematicides, have been targeted for market withdrawal. Though justified, these stricter regulations have limited the number of available nematicides to the point that for several nematode threats there are no control options^{27,28}. Despite the need for safer and more eco-friendly substitutes, only a handful of new small-molecule nematicides have been commercialized over the last 15 years³¹⁻³⁶. Unfortunately, similar problems exist in the animal health sector. Nematode resistance has developed against the majority of anthelmintic drugs that are used to treat infected livestock^{25,38-40}, which has cast doubt upon the long-term utility of an already limited pool of therapies.

Although sound agricultural practices, biologicals, and GMOs can reduce PPN infestation, synthetic small-molecule nematicides have been a significant part of a complete crop protection strategy for decades^{21,22,25-27}. In recent years, however, many effective synthetic nematicides have been restricted or banned due to adverse effects on human health and the environment²⁸⁻³¹. For example, the ozone-depleting fumigant methyl bromide, as well as many of the neurotoxic organophosphate and carbamate nematicides, have been targeted for market withdrawal. Though justified, these stricter regulations have limited the number of available nematicides to the point that for several nematode threats there are no control options^{27,28}. Despite the need for safer and more eco-friendly substitutes, only a handful of new small-molecule nematicides have been commercialized over the last 15 years³¹⁻³⁶. Unfortunately, similar problems exist in the animal health sector. Nematode resistance has developed against the majority of anthelmintic drugs that are used to treat infected livestock^{25,38-40}, which has cast doubt upon the long-term utility of an already limited pool of therapies.

Safer and less environmentally toxic nematicides are desperately needed to replace those that are being withdrawn from the market, and new anthelmintics with novel modes-of-action are also needed to combat the growing resistance to currently used treatments.

### SUMMARY

In some embodiments of the present application, compounds of the application were shown to inhibit the movement of infective larvae from the plant-parasitic root-knot nematode species Meloidgyne incognita and Meloidogyne chitwoodiin vitro as well as to inhibit egg-hatching of the root-knot nematode Meloidogyne hapla. In soil-based experiments, which more accurately simulate real-world conditions, it was shown that compounds of the application can inhibit the infection of tomato plant roots by the plant-parasitic nematodes (PPNs) Meloidogyne incognita and Meloidogyne chitwoodi. Finally, it was shown that compounds of Formula I may be selectively active against PPNs, as in some embodiments of the application, compounds of Formula I were relatively inactive against human HepG2 cells and non-lethal to zebrafish at concentrations up to 45 μM. In contrast, the commercial nematicides, tioxazafen has an LC₅₀ value in zebrafish of at most 15 μM, suggesting that it is at least 3 times more potent at killing fish. Altogether, the results reported herein suggest that compounds of the application can be similarly effective as commercial nematicides against PPNs in soil-based infection assays, but have selectivity for parasitic nematodes that is comparable to, or better than, commercially used compounds.

Accordingly, the present application includes a method for treating or preventing a plant nematode infection comprising administering to a plant in need thereof, an effective amount of one or more compounds of Formula (1) and/or salts and/or solvates thereof,

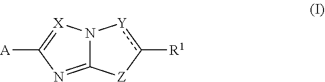

wherein:
[symbol] is a single bond or a double bond;
X is CR² or N;
Y is CR³ or N, when [symbol] is a double bond, or
Y is CHR³ or NR⁴ when [symbol] is a single bond;
Z is O or S; [symbol]
A is a 6 or 10 membered aryl or a 5, 6, 8, 9 or 10 membered heteroaryl that is unsubstituted or substituted with one to five substituents independently selected from halo, cyano, NH₂, CO₂H, C(O)H, C₁₋₄alkyl, C(O)C₁₋₄alkyl, CO₂C₁₋₄alkyl, OC(O)C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), OC₁₋₄alkyleneOC₁₋₄alkyl, C₁₋₄alkyleneOC₁₋₄alkyl and OC₁₋₄alkyl;
R¹, R² and R³ are independently selected from H, halo, cyano, NH₂, CO₂H, C(O)H, C₁₋₄alkyl, C(O)C₁₋₄alkyl, CO₂C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, OC₁₋₄alkyl, OC₁₋₄alkyleneOC₁₋₄alkyl, C₃₋₆cycloalkyl and C₃₋₆heterocycloalkyl;
R⁴ is selected from H, C₁₋₄alkyl, C(O)C₁₋₄alkyl, CO₂C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, C₃₋₆cycloalkyl and C₃₋₆heterocycloalkyl, and
each alkyl group in the compound of Formula I is optionally halo-substituted, provided that:
when A is unsubstituted phenyl or phenyl monosubstituted on the 4-position with halo, Z is S and Y is CR³, then at least one of R¹—R⁴ is other than H and R¹ and/or R³ are not halo, C₁₋₄alkyl, NH₂ or C(O)H; and
when A is pyridinyl or pyrimidinyl, Y is CR² and [symbol] is a double bond, then R¹ and R² are each not C(O)NH₂, C(O)NHC₁₋₄alkyl or C(O)NHC₁₋₄fluoroalkyl.

The present application also includes a method of treating or preventing a disease, disorder or condition in arising from a plant nematode infection comprising administering an effective amount of one or more compounds of the application and/or salts and/or solvates thereof to a plant in need thereof.

The present application also includes a composition comprising one or more carriers and one or more compounds of the application and/or salts and/or solvates thereof. In some embodiments, the composition is a agricultural composition. Accordingly, the present application also includes a agricultural composition comprising one or more pharmaceutically acceptable carriers and one or more compounds of the application and/or salts and/or solvates thereof in an amount effective to treat or prevent a plant nematode infection or to treat or prevent a disease, disorder or condition in arising from a plant nematode infection.

The present application includes a method of treating or preventing a plant nematode infection or a disease, a disorder, or a condition arising from a nematode infection comprising administering one or more compositions of the application to a plant in need thereof.

The present application includes all novel compounds of Formula I and/or salts and/or solvates thereof. In some embodiments, the present application also includes a compound of Formula I-F or a salt and/or solvate thereof:

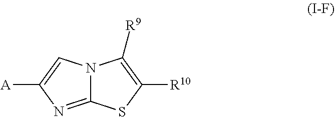

wherein
R⁹ is H or CH₃;
R¹⁰ is H or CH₃;
A¹ is

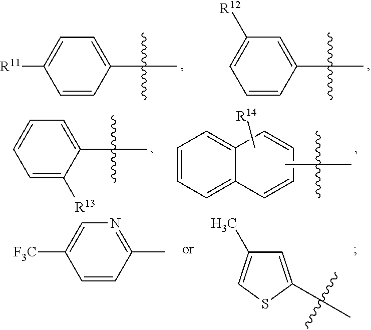

R¹¹ is C₁₋₄fluoroalkyl, OC₁₋₄fluoroalkyl, CN, CO₂C₁₋₄alkyl or C(O)C₁₋₄alkyl;
R¹² is F or I;
R¹³ is F, Cl or I; and
R¹⁴ is H or OC₁₋₄alkyl,
provided that:
when A¹ is

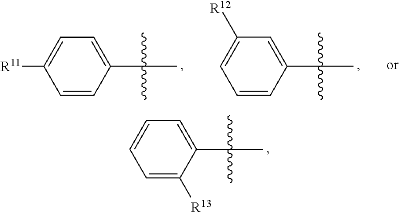

then R¹⁰ is CH₃; and
when R¹¹ is C₁₋₄fluoroalkyl or OC₁₋₄fluoroalkyl, then R⁹ is H.

In some embodiments, the compound of Formula I-F are selected from I-11, I-17, 1-21, I-33, I-34, I-50, I-51, I-52, I-53, I-54, I-56, I-58, I-73, I-75, I-81, I-82, I-83, I-160, I-161, I-162 and I-163, or a salt and/or solvate thereof.

The present application also includes novel compounds 1-28, I-39, I-42, I-43, 1-67, I-70, I-71, I-76 or I-77, or a salt and/or solvate thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DESCRIPTION

I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in this application, the singular forms “a”, “an” and “the” include plural references unless the content clearly dictates otherwise. For example, an embodiment including “a compound” should be understood to present certain aspects with compound or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “compound(s) of the application” and the like as used herein refers to a compound of Formula (1), (I-A), (I-B), (I-C), (I-D), (I-E), (I-F), (I-G), (I-H), (I-J), (I-K), (I-L), (I-M), (I-N), (I-O), (I-P), (I-Q), (I-R), (I-S) or (I-T) and/or salts and/or solvates thereof. Compounds of the application also include any of the novel compounds disclosed herein, and/or salts and/or solvates thereof.

The term “composition of the application” or “composition of the present application” and the like as used herein refers to a composition comprising one or more compounds of the application.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₄alkyl means an alkyl group having 1, 2, 3 or 4 carbon atoms.

The term “aryl” as used herein means carbocyclic groups containing at least one aromatic ring and contains either 6, 9 or 10 carbon atoms.

The term “cycloalkyl,” as used herein means a saturated carbocyclic group. The number of carbon atoms that are possible in the referenced cycloalkyl group are indicated by the numerical prefix “Cₙ₁₋ₙ₂”. For example, the term C₃₋₆cycloalkyl means a cycloalkyl group having 3, 4, 5 or 6 carbon atoms.

The term “heterocycloalkyl” as used herein means a non-aromatic cyclic group containing one or more heteroatoms selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 3, of the ring atoms is replaced with a heteroatom as defined above.

The term “heteroaryl” as used herein, whether it is used alone or as part of another group, means cyclic groups containing at least one heteroaromatic ring containing 5, 6, 8, 9 or 10 atoms in which one or more of the atoms are a heteroatom selected from O, S and N and the remaining atoms are C. When a heteroaryl group contains a numerical prefix this prefix indicates the number of carbon atoms in the corresponding carbocyclic group, in which one or more, suitably 1 to 5, of the ring atoms is replaced with a heteroatom as defined above. Heteroaryl groups are optionally benzofused.

The term “benzofused” as used herein refers to a bicyclic group in which a benzene ring is fused with another ring.

A first ring being “fused” with a second ring means the first ring and the second ring share two adjacent atoms there between.

The term “halo” or “halogen” as used herein refers to a halogen atom and includes fluoro, chloro, bromo and iodo.

The term “halo-substituted” as used herein means that one or more, including all, of the available hydrogen atoms in a referenced groups are substituted with a halogen atom.

The term “fluoro-substituted” as used herein means that one or more, including all, of the available hydrogen atoms in a referenced groups are substituted with a fluorine atom.

The term “available”, as in “available hydrogen atoms” refers to atoms that would be known to a person skilled in the art to be capable of replacement by another atom or group.

[symbol]

The symbol [symbol] over a chemical bond in a chemical group means that bond is the point of attachment of the chemical group to another chemical structure.

The term “solvate” as used herein means a compound, or a salt of a compound, wherein molecules of a suitable solvent are incorporated in the crystal lattice.

The term “nematode” as used herein refers to a worm of the phylum Nematoda.

The expression “disease, disorder or condition arising from a nematode infection” as used herein refers to any disease, disorder or condition that is directly or indirectly caused by the presence of a nematode infection in a plant.

The term “plant” as used herein refers to any species or genera of plant that may be the target of infection by a nematode. The term “plant” also refers to any part of the plant, including, for example, seeds, roots, stems, flowers and leaves.

The term “nematode infection” as used herein refers to an invasion of any part of a subject by a foreign undesirable nematode.

The term “anthelmintic” or “anthelmintics” as used herein refers to a group of antiparasitic drugs used in the treatment and prevention of nematode infections in animals.

As used herein, a compound with “nematicidal activity” or “nematicide” is a compound, which when tested, has measurable nematode-killing activity or results in sterility or reduced fertility in the nematodes such that fewer viable or no offspring result, or compromises the ability of the nematode to infect or reproduce in its host, or interferes with the growth or development of a nematode. The compound may also display nematode repellant properties.

The term “nematicidal composition” as used herein refers to a composition of matter for treating one or more nematode infections.

The term “carrier” as used herein means an inert compound with which the composition is mixed or formulated. The term “carrier” includes, for example, solid or liquid carriers or combinations thereof.

The term “administered”, “administering”, “application” or “applied” as used herein means administration of an effective amount of a compound, including compounds of the application, to a plant.

As used herein, the term “effective amount” or “therapeutically effective amount” means an amount effective, at dosages and for periods of time necessary to achieve a desired result. For example, in the context of treating a nematode infection, or a disease, disorder or condition arising from a nematode infection, an effective amount of a compound is an amount that, for example, reduces the nematode infection compared to the nematode infection without administration of the compound. By “reducing the infection”, it is meant, for example, reducing the amount of the infectious agent in the subject and/or reducing the symptoms of the infection. The amount of a given compound or composition that will correspond to such an amount will vary depending upon various factors, such as the given compound or composition, the formulation, the route of administration, the type of condition, disease or disorder, the identity of the plant being treated, and the like, but can nevertheless be routinely determined by one skilled in the art.

The terms “to treat”, “treating” and “treatment” as used herein and as is well understood in the art, means an approach for obtaining beneficial or desired results, including clinical results. Beneficial or desired clinical results include, but are not limited to, diminishment of extent of nematode infection, stabilization (i.e. not worsening) of the state of the nematode infection, preventing spread of the nematode infection, delay or slowing of infection progression, amelioration or palliation of the nematode infectious state, diminishment of the reoccurrence of nematode infection, diminishment, stabilization, alleviation or amelioration of one or more diseases, disorders or conditions arising from the nematode infection, diminishment of the reoccurrence of one or more diseases, disorders or conditions arising from the nematode infection, and remission of the nematode infection and/or one or more symptoms or conditions arising from the nematode infection, whether partial or total, whether detectable or undetectable. “To treat”, “treating” and “treatment” can also mean prolonging survival as compared to expected survival if not receiving treatment. “To treat”, “treating” and “treatment” as used herein also include prophylactic treatment. For example, a subject with an early nematode infection is treated to prevent progression, or alternatively a subject in remission is treated to prevent recurrence.

“Palliating” an infection, disease, disorder and/or condition means that the extent and/or undesirable manifestations of an infection, disease, disorder and/or condition are lessened and/or time course of the progression is slowed or lengthened, as compared to not treating the infection, disease, disorder and/or condition.

The term “prevention” or “prophylaxis” and the like as used herein refers to a reduction in the risk or probability of a subject becoming afflicted with a nematode infection and/or a disease, disorder and/or condition arising from a nematode infection or manifesting a symptom associated with a nematode infection and/or a disease, disorder and/or condition arising from a nematode infection.

II. Methods and Uses of the Application

The present application includes a method for treating or preventing a plant nematode infection comprising administering to a plant in need thereof, an effective amount of one or more compounds of Formula (1) and/or salts and/or solvates thereof,

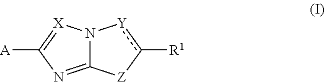

wherein:
[symbol] is a single bond or a double bond;
X is CR² or N;
Y is CR³ or N, when [symbol] is a double bond, or
Y is CHR³ or NR⁴ when [symbol] is a single bond;
Z is O or S;
A is a 6 or 10 membered aryl or a 5, 6, 8, 9 or 10 membered heteroaryl that is unsubstituted or substituted with one to five substituents independently selected from halo, cyano, NH₂, CO₂H, C(O)H, C₁₋₄alkyl, C(O)C₁₋₄alkyl, CO₂C₁₋₄alkyl, OC(O)C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), OC₁₋₄alkyleneOC₁₋₄alkyl, C₁₋₄alkyleneOC₁₋₄alkyl and OC₁₋₄alkyl;
R¹, R² and R³ are independently selected from H, halo, cyano, NH₂, CO₂H, C(O)H, C₁₋₄alkyl, C(O)C₁₋₄alkyl, CO₂C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, OC₁₋₄alkyl, OC₁₋₄alkyleneOC₁₋₄alkyl, C₃₋₆cycloalkyl and C₃₋₆heterocycloalkyl;
R⁴ is selected from H, C₁₋₄alkyl, C(O)C₁₋₄alkyl, CO₂C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, C₃₋₆cycloalkyl and C₃₋₆heterocycloalkyl, and
each alkyl group in the compound of Formula I is optionally halo-substituted, provided that:
when A is unsubstituted phenyl or phenyl monosubstituted on the 4-position with halo, Z is S and Y is CR³, then at least one of R¹—R⁴ is other than H and R¹ and/or R³ are not halo, C₁₋₄alkyl, NH₂ or C(O)H; and
when A is pyridinyl or pyrimidinyl, Y is CR² and [symbol] is a double bond, then R¹ and R² are each not C(O)NH₂, C(O)NHC₁₋₄alkyl or C(O)NHC₁₋₄fluoroalkyl.

The application also includes a use of one or more compounds of the application and/or salts and/or solvates thereof for treating or preventing a nematode infection. The application further includes one or more compounds of the application and/or salts and/or solvates thereof for use for treating or preventing a nematode infection.

The present application also includes a method of treating or preventing a disease, disorder or condition arising from a nematode infection comprising administering an effective amount of one or more compounds of the application and/or salts and/or solvates thereof to a subject in need thereof.

The application also includes a use of one or more compounds of the application and/or salts and/or solvates thereof for treating or preventing a disease, disorder or condition arising from a nematode infection. The application further includes one or more compounds of the application and/or salts and/or solvates thereof for use for treating or preventing a disease, disorder or condition arising from a nematode infection.
In some embodiments, [symbol] is double bond.
In some embodiments, X is CR².
In some embodiments Y is CR³. In some embodiments, Y is N.
In some embodiments, Z is S. In some embodiments, X is O.
In some embodiments, Y is CHR³.

In some embodiments, each alkyl group in the compound of Formula I is optionally fluoro-substituted. In some embodiments, when an alkyl group is halo-substituted and the halo is Cl, Br, and/or I, only one, two or three of the hydrogen atoms in the alkyl group are substituted with the halogen atom.

In some embodiments, A is a 6 or 10 membered aryl that is unsubstituted or substituted. In some embodiments, A is phenyl or naphthyl each of which is unsubstituted or substituted. In some embodiments, A is unsubstituted or substituted phenyl or unsubstituted or substituted naphthyl. In some embodiments, A is unsubstituted or substituted phenyl. In some embodiments, A is substituted phenyl or substituted naphthyl. In some embodiments, A is substituted phenyl. In some embodiments A is unsubstituted phenyl.

In some embodiments, when A is a substituted 6 or 10 membered aryl, the substituents are independently selected from one to three of halo, cyano, NH₂, CO₂H, C(O)H, C₁₋₄alkyl, C(O)C₁₋₄alkyl, CO₂C₁₋₄alkyl, OC(O)C₁₋₄alkyl NHC₁₋₄alkyl, NHC(O)C₁₋₄alkyl, C(O)NH₂, C(O)NHC₁₋₄alkyl, OC₁₋₄alkyleneOC₁₋₄alkyl, C₁₋₄alkyleneOC₁₋₄alkyl and OC₁₋₄alkyl, and each alkyl group is optionally fluoro-substituted. In some embodiments, when A is a substituted 6 or 10 membered aryl, the substituents on A are independently selected from one or two of halo, cyano, NH₂, CO₂H, C(O)H, CH₃, CF₃, CHF₂, OCH₃, OCF₃, OCHF₂, CH₂CH₃, OCH₂CH₃, CH(CH₃)₂, C(CH₃)₃, C(O)CH₃, C(O)CH₂CH₃, C(O)OCH₃, NHCH₃, NHC(O)CH₃, NHC(O)CH₂CH₃, C(O)NH₂, C(O)NHCH₃, CH₂OCH₃ and OCH₂OCH₃. In some embodiments, when A is a substituted 6 or 10 membered aryl, the substituents on A are independently selected from one or two of Cl, F, Br, CH₃, CF₃, CH₂CH₃, OCH₃, OCF₃, OCHF₂ and OCH₂CH₃. In some embodiments, when A is a substituted 6 or 10 membered aryl, the substituents on A are independently selected from one of Cl, F, Br, CH₃, CF₃, CH₂CH₃, OCH₃, OCF₃, OCHF₂ and OCH₂CH₃.

In some embodiments, A is a 5, 6, 8, 9 or 10 membered heteroaryl that is unsubstituted or substituted. In some embodiments, the heteroaryl is selected from pyrrolyl, furanyl, imidazolyl, thienyl, oxazolyl, isoxazolyl, pyrazolyl, thiazolyl, isothiazolyl, oxadiazolyl, isothiazolyl, thiadiazolyl, pyridinyl, pyrazinyl, pyridazinyl, pyrimidinyl, quinolinyl and isoquinolinyl, each of which is unsubstituted or substituted. In some embodiments, the heteroaryl is selected from pyrrolyl, furanyl, imidazolyl, thienyl, oxazolyl, isoxazolyl, pyrazolyl, thiazolyl, isothiazolyl, pyridinyl, pyrazinyl, pyridazinyl and pyrimidinyl, each of which is unsubstituted or substituted. In some embodiments, the heteroaryl is selected from thien-2-yl,thien-3-yl, pyridine-2yl and pyridine-3-yl, each of which is unsubstituted or substituted. In some embodiments, the heteroaryl is selected from quinolinyl, quinoxalinyl, quinazolinyl, indolyl, benzofuranyl, benzothiophenyl, benzooxazolyl, benzothiazolyl, benzimidazolyl, imidazopyridinyl, pyrrolopyridinyl, pyrrolopyrazinyl, pyrroloimidazolyl, pyrrolopyrrolyl, imidazoimidazolyl, and imidazothiazolyl, each of which is unsubstituted or substituted.

In some embodiments, when A is a substituted 5, 6, 8, 9 or 10 membered heteroaryl, the substituents are independently selected from one to three of halo, cyano, NH₂, CO₂H, C(O)H, C₁₋₄alkyl, C(O)C₁₋₄alkyl, CO₂C₁₋₄alkyl, OC(O)C₁₋₄alkyl, NHC₁₋₄alkyl, NHC(O)C₁₋₄alkyl, C(O)NH₂, C(O)NHC₁₋₄alkyl, OC₁₋₄alkyleneOC₁₋₄alkyl, C₁₋₄alkyleneOC₁₋₄alkyl and OC₁₋₄alkyl, and each alkyl group is optionally fluoro-substituted. In some embodiments, when A is a substituted 5, 6, 8, 9 or 10 membered heteroaryl, the substituents on A are independently selected from one or two of halo, cyano, NH₂, CO₂H, C(O)H, CH₃, CF₃, CHF₂, OCH₃, OCF₃, OCHF₂, CH₂CH₃, OCH₂CH₃, CH(CH₃)₂, C(CH₃)₃, C(O)CH₃, C(O)CH₂CH₃, C(O)OCH₃, NHCH₃, NHC(O)CH₃, NHC(O)CH₂CH₃, C(O)NH₂, C(O)NHCH₃, CH₂OCH₃ and OCH₂OCH₃. In some embodiments, when A is a substituted 5, 6 or 10 heteroaryl, the substituents on A are independently selected from one or two of Cl, F, Br, CH₃, CF₃, CH₂CH₃, OCH₃, OCF₃, OCHF₂ and OCH₂CH₃. In some embodiments, when A is a substituted 5, 6 or 10 heteroaryl, the substituents on A are independently selected from one of Cl, F, CH₃ and CF₃.

[symbol]

In some embodiments, X is CR² and Y is CR³ (and therefore, is a double bond) and R¹, R² and R³ are independently selected from H, Cl, Br, I, cyano, NH₂, CO₂H, C(O)H, CH₃, CF₃, CHF₂, OCH₃, OCF₃, OCHF₂, CH₂CH₃, OCH₂CH₃, CH(CH₃)₂, C(CH₃)₃, C(O)CH₃, C(O)CH₂CH₃, C(O)OCH₃, NHCH₃, NHC(O)CH₃, NHC(O)CH₂CH₃, C(O)NH₂, C(O)NHCH₃, CH₂OCH₃, OCH₂OCH₃, cyclopentyl, cyclohexyl, pyrolidin-1yl and piperidin-1-yl. In some embodiments, X is CR² and Y is CR³ and R¹, R² and R³ are independently selected from H, Cl, Br, cyano, NH₂, CO₂H, C(O)H, CH₃, CF₃, CHF₂, OCH₃, OCF₃, OCHF₂, CH₂CH₃, OCH₂CH₃, CH(CH₃)₂, C(CH₃)₃, C(O)CH₃, C(O)CH₂CH₃, C(O)OCH₃, NHCH₃, NHC(O)CH₃, NHC(O)CH₂CH₃, C(O)NH₂, C(O)NHCH₃, CH₂OCH₃, OCH₂OCH₃, pyrolidin-1yl and piperidin-1-yl. In some embodiments, X is CR² and Y is CR³ and R¹, R² and R³ are all H. In some embodiments, X is CR² and Y is CR³ and R² is other than H and R¹ and R³ are both H. In some embodiments, X is CR² and Y is CR³ and R² is selected from C₁₋₄ alkyl, NH₂ and C(O)H and R¹ and R³ are both H. In some embodiments, X is CR² and Y is CR³ and R¹ is other than H and R² and R³ are both H. In some embodiments, X is CR² and Y is CR³ and R¹ is selected from halo and C₁₋₄alkyl and R² and R³ are both H.

In some embodiments, Y is NR⁴ (and therefore [symbol] is a single bond) and R⁴ is selected from H, CH₃, CF₃, CHF₂, CH₂CH₃, CH(CH₃)₂, C(CH₃)₃, C(O)CH₃ and C(O)CH₂CH₃. In some embodiments, Y is NR⁴ and R⁴ is selected from H, CH₃, CF₃, CHF₂, CH₂CH₃, CH(CH₃)₂ and C(CH₃)₃. In some embodiments, Y is NR⁴ and R⁴ is selected from H and CH₃.

In some embodiments, the one or more compounds of Formula I have the following structure:

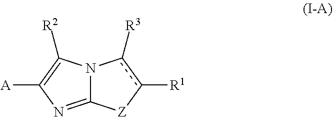

wherein A, R¹, R², R³, Z and [symbol] are as defined in Formula I, including all embodiments thereof. In some embodiments, Z is S in the compounds of Formula I-A.

In some embodiments, the one or more compounds of Formula I have the following structure:

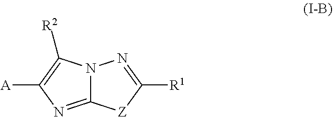

wherein A, R¹, R² and Z as defined in Formula I, including all embodiments thereof. In some embodiments, Z is S in the compounds of Formula I-B.

In some embodiments, the one or more compounds of Formula I have the following structure:

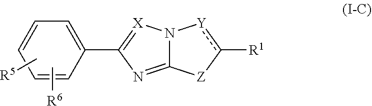

wherein R¹, X, Y, Z and [symbol] are as defined in Formula I, including all embodiments thereof, and R⁵ and R⁶ are selected from H, halo, CN, CH₃, CF₃, OCH₃, OCF₃, OCHF₂, OCH₂CH₃, C(O)CH₃, C(O)OCH₃, C(O)CH₂CH₃, C(O)OCH₃, NH₂, NHC(O)CH₃ and C(O)H, provided that one of R⁵ and R⁶ is not H and when R⁵ or R⁶ is at the para position of the phenyl ring, it is not halo.

In some embodiments, the one or more compounds of Formula I have the following structure:

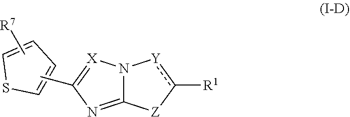

wherein R¹, X, Y, Z and [symbol] are as defined in Formula I, including all embodiments thereof, and R⁷ is selected from H, halo and C₁₋₄alkyl.

In some embodiments, the one or more compounds of Formula I have the following structure:

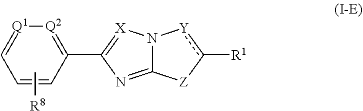

wherein R¹, X, Y, Z and [symbol] are as defined in Formula I, including all embodiments thereof, one of Q1 and Q² is N and the other is C and R⁸ is selected from H, halo, CN, CH₃, CF₃, OCH₃, OCF₃, OCHF₂, OCH₂CH₃, C(O)CH₃, C(O)OCH₃, C(O)CH₂CH₃, C(O)OCH₃, NH₂, NHC(O)CH₃ and C(O)H. In some embodiments, R⁸ is selected from H, Cl and F. In some embodiments, R⁸ is a Cl at the position para to the bond to the remaining portion of the compound of Formula I-E.

In some embodiments, the one or more compounds Formula (I) are selected from:

| Cpd | |
|---|---|
| No. | Structure |
| I-1 | 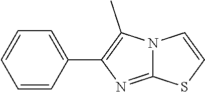 |
| I-2 | 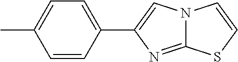 |
| I-3 | 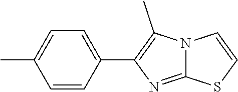 [SMILES: Cc1ccc(-c2nc3sccn3c2C)cc1] |
| I-4 | 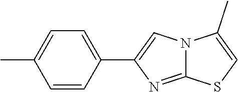 [SMILES: Cc1ccc(-c2cn3c(C)csc3n2)cc1] |
| I-5 | 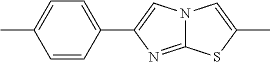 |
| I-6 | 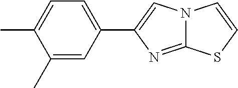 [SMILES: Cc1ccc(-c2cn3ccsc3n2)cc1C] |
| I-7 | 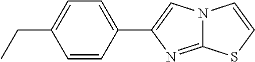 [SMILES: CCc1ccc(-c2cn3ccsc3n2)cc1] |
| I-8 | 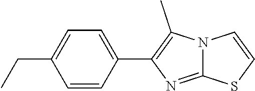 [SMILES: CCc1ccc(-c2nc3sccn3c2C)cc1] |
| I-9 | 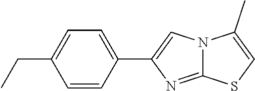 [SMILES: CCc1ccc(-c2cn3c(C)csc3n2)cc1] |
| I-10 | 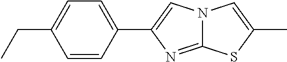 [SMILES: CCc1ccc(-c2cn3cc(C)sc3n2)cc1] |
| I-11 | 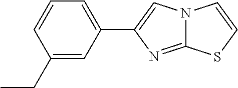 [SMILES: CCc1cccc(-c2cn3ccsc3n2)c1] |
| I-12 | 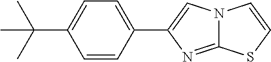 [SMILES: CC(C)(C)c1ccc(-c2cn3ccsc3n2)cc1] |
| I-13 | 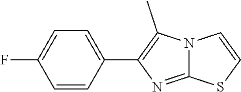 [SMILES: Cc1c(-c2ccc(F)cc2)nc2sccn12] |
| I-14 | 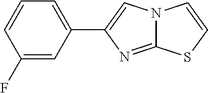 [SMILES: Fc1cccc(-c2cn3ccsc3n2)c1] |
| I-15 | 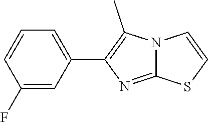 [SMILES: Cc1c(-c2cccc(F)c2)nc2sccn12] |
| I-16 | 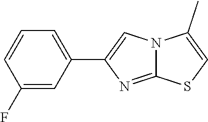 [SMILES: Cc1csc2nc(-c3cccc(F)c3)cn12] |
| I-17 | 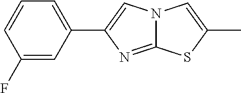 [SMILES: Cc1cn2cc(-c3cccc(F)c3)nc2s1] |
| I-18 | 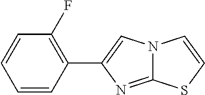 [SMILES: Fc1ccccc1-c1cn2ccsc2n1] |
| I-19 | 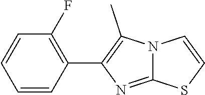 [SMILES: Cc1c(-c2ccccc2F)nc2sccn12] |
| I-20 | 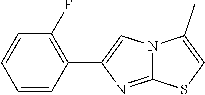 [SMILES: Cc1csc2nc(-c3ccccc3F)cn12] |
| I-21 | 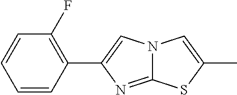 [SMILES: Cc1cn2cc(-c3ccccc3F)nc2s1] |
| I-22 | 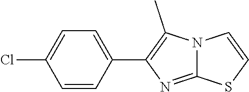 [SMILES: Cc1c(-c2ccc(Cl)cc2)nc2sccn12] |
| I-23 | 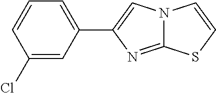 [SMILES: Clc1cccc(-c2cn3ccsc3n2)c1] |
| I-24 | 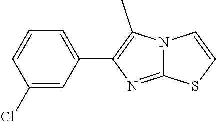 [SMILES: Cc1c(-c2cccc(Cl)c2)nc2sccn12] |
| I-25 | 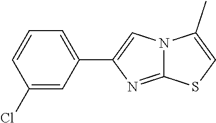 [SMILES: Cc1csc2nc(-c3cccc(Cl)c3)cn12] |
| I-26 | 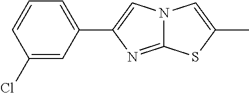 [SMILES: Cc1cn2cc(-c3cccc(Cl)c3)nc2s1] |
| I-27 | 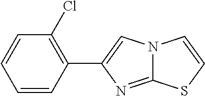 [SMILES: Clc1ccccc1-c1cn2ccsc2n1] |
| I-28 | 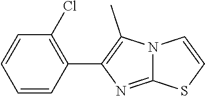 [SMILES: Cc1c(-c2ccccc2Cl)nc2sccn12] |
| I-29 | 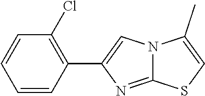 [SMILES: Cc1csc2nc(-c3ccccc3Cl)cn12] |
| I-30 | 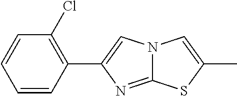 [SMILES: Cc1cn2cc(-c3ccccc3Cl)nc2s1] |
| I-31 | 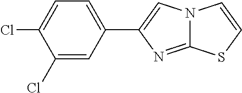 [SMILES: Clc1ccc(-c2cn3ccsc3n2)cc1Cl] |
| I-32 | 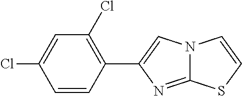 [SMILES: Clc1ccc(-c2cn3ccsc3n2)c(Cl)c1] |
| I-33 | 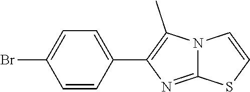 [SMILES: Cc1c(-c2ccc(Br)cc2)nc2sccn12] |
| I-34 | 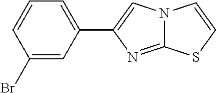 [SMILES: Brc1cccc(-c2cn3ccsc3n2)c1] |
| I-35 | 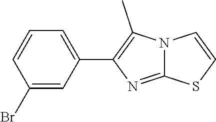 [SMILES: Cc1c(-c2cccc(Br)c2)nc2sccn12] |
| I-36 | 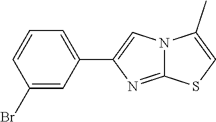 [SMILES: Cc1csc2nc(-c3cccc(Br)c3)cn12] |
| I-37 | 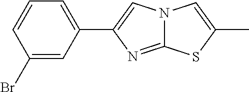 [SMILES: Cc1cn2cc(-c3cccc(Br)c3)nc2s1] |
| I-38 | 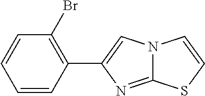 [SMILES: Brc1ccccc1-c1cn2ccsc2n1] |
| I-39 | 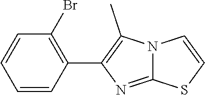 [SMILES: Cc1c(-c2ccccc2Br)nc2sccn12] |
| I-40 | 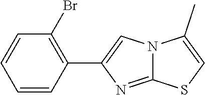 [SMILES: Cc1csc2nc(-c3ccccc3Br)cn12] |
| I-41 | 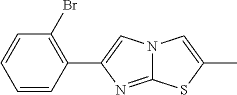 [SMILES: Cc1cn2cc(-c3ccccc3Br)nc2s1] |
| I-42 | 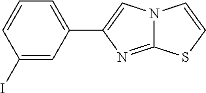 [SMILES: Ic1cccc(-c2cn3ccsc3n2)c1] |
| I-43 | 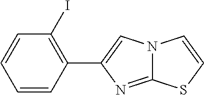 [SMILES: Ic1ccccc1-c1cn2ccsc2n1] |
| I-44 | 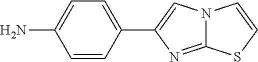 [SMILES: Nc1ccc(-c2cn3ccsc3n2)cc1] |
| I-45 | 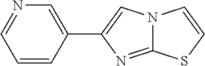 [SMILES: c1cncc(-c2cn3ccsc3n2)c1] |
| I-46 | 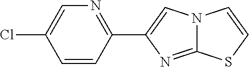 [SMILES: Clc1ccc(-c2cn3ccsc3n2)nc1] |
| I-47 | 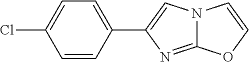 [SMILES: Clc1ccc(-c2cn3ccoc3n2)cc1] |
| I-48 | 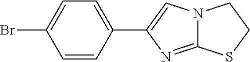 [SMILES: Brc1ccc(-c2cn3c(n2)SCC3)cc1] |
| I-49 | 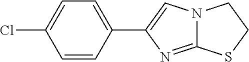 [SMILES: Clc1ccc(-c2cn3c(n2)SCC3)cc1] |
| I-50 | 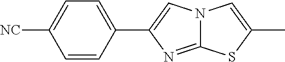 [SMILES: Cc1cn2cc(-c3ccc(C#N)cc3)nc2s1] |
| I-51 | 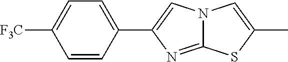 [SMILES: Cc1cn2cc(-c3ccc(C(F)(F)F)cc3)nc2s1] |
| I-52 | 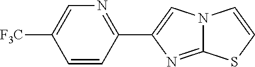 [SMILES: FC(F)(F)c1ccc(-c2cn3ccsc3n2)nc1] |
| I-53 | 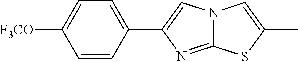 [SMILES: Cc1cn2cc(-c3ccc(OC(F)(F)F)cc3)nc2s1] |
| I-54 | 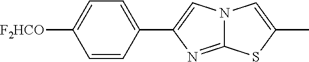 [SMILES: Cc1cn2cc(-c3ccc(OC(F)F)cc3)nc2s1] |
| I-55 | 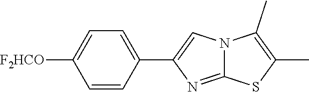 [SMILES: Cc1sc2nc(-c3ccc(OC(F)F)cc3)cn2c1C] |
| I-56 | 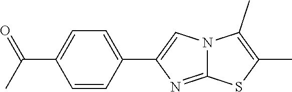 [SMILES: CC(=O)c1ccc(-c2cn3c(C)c(C)sc3n2)cc1] |
| I-57 | 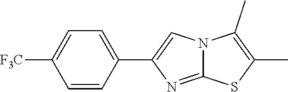 [SMILES: Cc1sc2nc(-c3ccc(C(F)(F)F)cc3)cn2c1C] |
| I-58 | 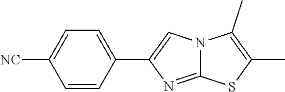 [SMILES: Cc1sc2nc(-c3ccc(C#N)cc3)cn2c1C] |
| I-59 | 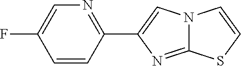 [SMILES: Fc1ccc(-c2cn3ccsc3n2)nc1] |
| I-60 | 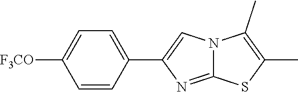 [SMILES: Cc1sc2nc(-c3ccc(OC(F)(F)F)cc3)cn2c1C] |
| I-61 | 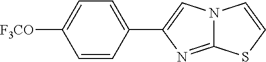 [SMILES: FC(F)(F)Oc1ccc(-c2cn3ccsc3n2)cc1] |
| I-62 | 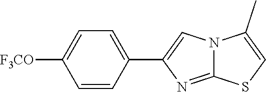 [SMILES: Cc1csc2nc(-c3ccc(OC(F)(F)F)cc3)cn12] |
| I-63 | 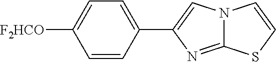 [SMILES: FC(F)Oc1ccc(-c2cn3ccsc3n2)cc1] |
| I-64 | 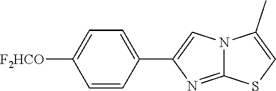 [SMILES: Cc1csc2nc(-c3ccc(OC(F)F)cc3)cn12] |
| I-65 | 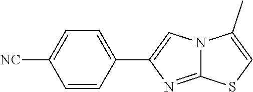 [SMILES: Cc1csc2nc(-c3ccc(C#N)cc3)cn12] |
| I-66 | 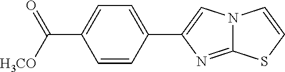 [SMILES: COC(=O)c1ccc(-c2cn3ccsc3n2)cc1] |
| I-67 | 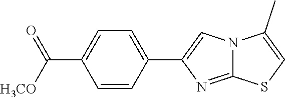 [SMILES: COC(=O)c1ccc(-c2cn3c(C)csc3n2)cc1] |
| I-68 | 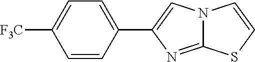 [SMILES: FC(F)(F)c1ccc(-c2cn3ccsc3n2)cc1] |
| I-69 | 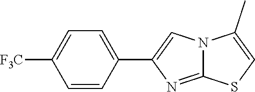 [SMILES: Cc1csc2nc(-c3ccc(C(F)(F)F)cc3)cn12] |
| I-70 | 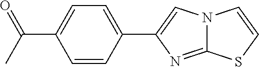 [SMILES: CC(=O)c1ccc(-c2cn3ccsc3n2)cc1] |
| I-71 | 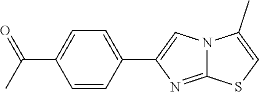 [SMILES: CC(=O)c1ccc(-c2cn3c(C)csc3n2)cc1] |
| I-72 | 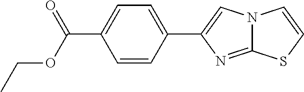 [SMILES: CCOC(=O)c1ccc(-c2cn3ccsc3n2)cc1] |
| I-73 | 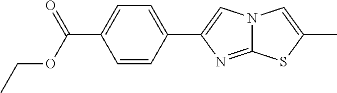 [SMILES: CCOC(=O)c1ccc(-c2cn3cc(C)sc3n2)cc1] |
| I-74 | 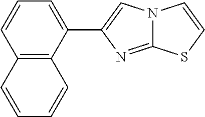 [SMILES: c1ccc2c(-c3cn4ccsc4n3)cccc2c1] |
| I-75 | 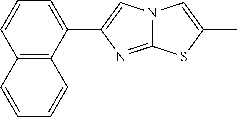 [SMILES: Cc1cn2cc(-c3cccc4ccccc34)nc2s1] |
| I-76 | 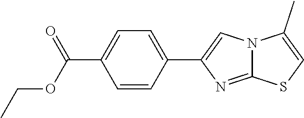 [SMILES: CCOC(=O)c1ccc(-c2cn3c(C)csc3n2)cc1] |
| I-77 | 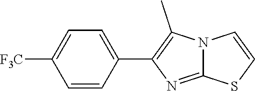 [SMILES: Cc1c(-c2ccc(C(F)(F)F)cc2)nc2sccn12] |
| I-78 | 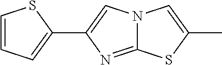 |
| I-79 | 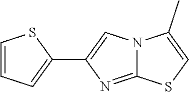 |
| I-80 | 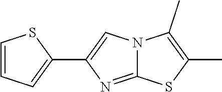 |
| I-81 | 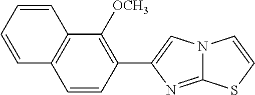 [SMILES: COc1c(-c2cn3ccsc3n2)ccc2ccccc12] |
| I-82 | 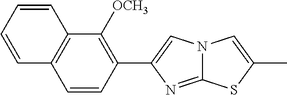 [SMILES: COc1c(-c2cn3cc(C)sc3n2)ccc2ccccc12] |
| I-83 | 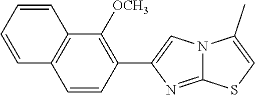 [SMILES: COc1c(-c2cn3c(C)csc3n2)ccc2ccccc12] |
| I-84 | 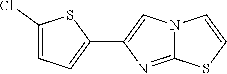 [SMILES: Clc1ccc(-c2cn3ccsc3n2)s1] |
| I-85 | 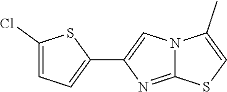 |
| I-86 | 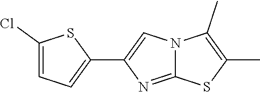 [SMILES: Cc1sc2nc(-c3ccc(Cl)s3)cn2c1C] |
| I-87 | 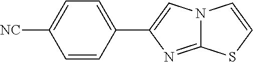 [SMILES: N#Cc1ccc(-c2cn3ccsc3n2)cc1] |
| I-88 | 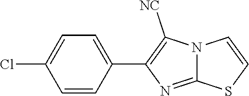 [SMILES: N#Cc1c(-c2ccc(Cl)cc2)nc2sccn12] |
| I-89 | 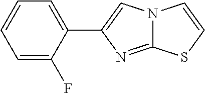 [SMILES: Fc1ccccc1-c1cn2ccsc2n1] |
| I-90 | 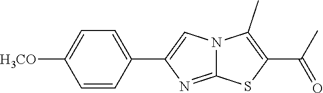 [SMILES: COc1ccc(-c2cn3c(C)c(C(C)=O)sc3n2)cc1] |
| I-91 | 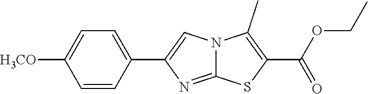 [SMILES: CCOC(=O)c1sc2nc(-c3ccc(OC)cc3)cn2c1C] |
| I-92 | 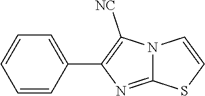 [SMILES: N#Cc1c(-c2ccccc2)nc2sccn12] |
| I-93 | 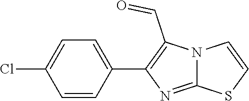 [SMILES: O=Cc1c(-c2ccc(Cl)cc2)nc2sccn12] |
| I-94 | 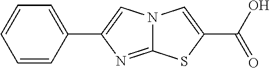 [SMILES: O=C(O)c1cn2cc(-c3ccccc3)nc2s1] |
| I-95 | 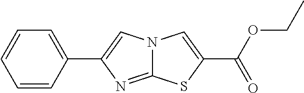 [SMILES: CCOC(=O)c1cn2cc(-c3ccccc3)nc2s1] |
| I-96 | 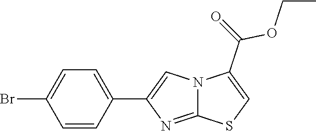 [SMILES: CCOC(=O)c1csc2nc(-c3ccc(Br)cc3)cn12] |
| I-97 | 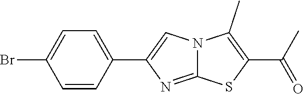 [SMILES: CC(=O)c1sc2nc(-c3ccc(Br)cc3)cn2c1C] |
| I-98 | 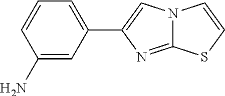 [SMILES: Nc1cccc(-c2cn3ccsc3n2)c1] |
| I-99 | 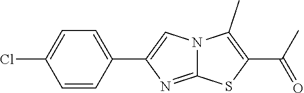 [SMILES: CC(=O)c1sc2nc(-c3ccc(Cl)cc3)cn2c1C] |
| I-100 | 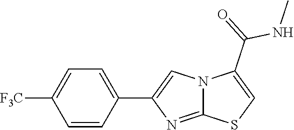 [SMILES: CNC(=O)c1csc2nc(-c3ccc(C(F)(F)F)cc3)cn12] |
| I-101 | 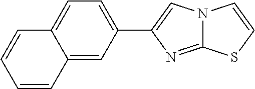 [SMILES: c1ccc2cc(-c3cn4ccsc4n3)ccc2c1] |
| I-102 | 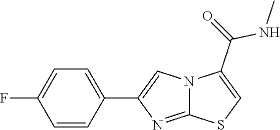 [SMILES: CNC(=O)c1csc2nc(-c3ccc(F)cc3)cn12] |
| I-103 | 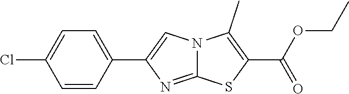 [SMILES: CCOC(=O)c1sc2nc(-c3ccc(Cl)cc3)cn2c1C] |
| I-104 | 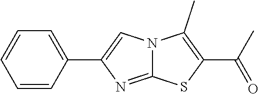 [SMILES: CC(=O)c1sc2nc(-c3ccccc3)cn2c1C] |
| I-105 | 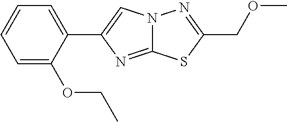 [SMILES: CCOc1ccccc1-c1cn2nc(COC)sc2n1] |
| I-106 | 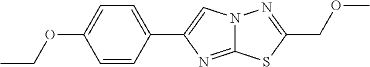 [SMILES: CCOc1ccc(-c2cn3nc(COC)sc3n2)cc1] |
| I-107 | 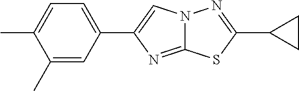 [SMILES: Cc1ccc(-c2cn3nc(C4CC4)sc3n2)cc1C] |
| I-108 | 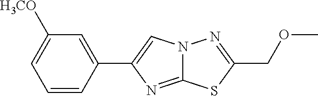 [SMILES: COCc1nn2cc(-c3cccc(OC)c3)nc2s1] |
| I-109 | 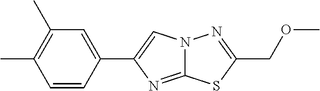 |
| I-110 | 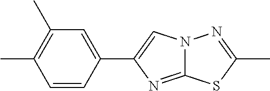 |
| I-111 | 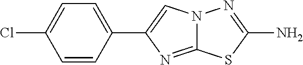 [SMILES: Nc1nn2cc(-c3ccc(Cl)cc3)nc2s1] |
| I-112 | 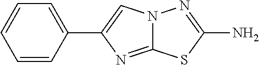 [SMILES: Nc1nn2cc(-c3ccccc3)nc2s1] |
| I-113 | 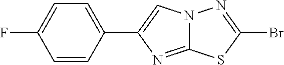 [SMILES: Fc1ccc(-c2cn3nc(Br)sc3n2)cc1] |
| I-114 | 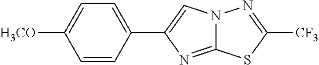 [SMILES: COc1ccc(-c2cn3nc(C(F)(F)F)sc3n2)cc1] |
| I-115 | 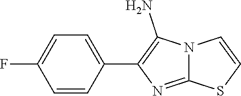 [SMILES: Nc1c(-c2ccc(F)cc2)nc2sccn12] |
| I-116 | 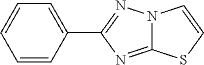 [SMILES: c1ccc(-c2nc3sccn3n2)cc1] |
| I-117 | 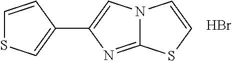 [SMILES: c1cc(-c2cn3ccsc3n2)cs1 ; Br] |
| I-118 | 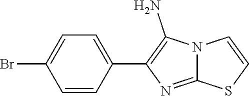 [SMILES: Nc1c(-c2ccc(Br)cc2)nc2sccn12] |
| I-119 | 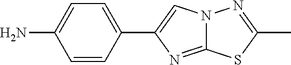 [SMILES: Cc1nn2cc(-c3ccc(N)cc3)nc2s1] |
| I-120 | 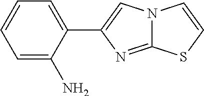 [SMILES: Nc1ccccc1-c1cn2ccsc2n1] |
| I-121 | 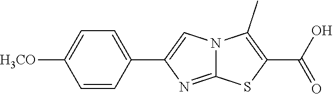 [SMILES: COc1ccc(-c2cn3c(C)c(C(=O)O)sc3n2)cc1] |
| I-122 | 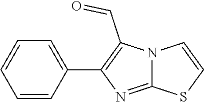 [SMILES: O=Cc1c(-c2ccccc2)nc2sccn12] |
| I-123 | 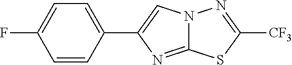 [SMILES: Fc1ccc(-c2cn3nc(C(F)(F)F)sc3n2)cc1] |
| I-124 | 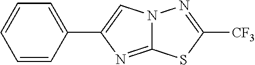 [SMILES: FC(F)(F)c1nn2cc(-c3ccccc3)nc2s1] |
| I-125 | 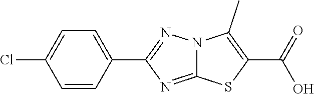 [SMILES: Cc1c(C(=O)O)sc2nc(-c3ccc(Cl)cc3)nn12] |
| I-126 | 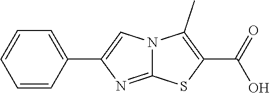 [SMILES: Cc1c(C(=O)O)sc2nc(-c3ccccc3)cn12] |
| I-127 | 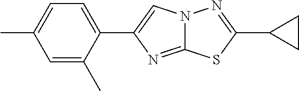 [SMILES: Cc1ccc(-c2cn3nc(C4CC4)sc3n2)c(C)c1] |
| I-128 | 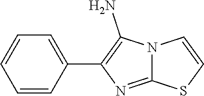 [SMILES: Nc1c(-c2ccccc2)nc2sccn12] |
| I-129 | 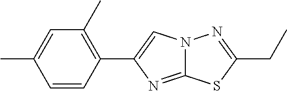 [SMILES: CCc1nn2cc(-c3ccc(C)cc3C)nc2s1] |
| I-130 | 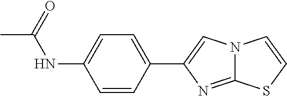 [SMILES: CC(=O)Nc1ccc(-c2cn3ccsc3n2)cc1] |
| I-131 | 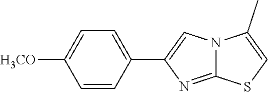 [SMILES: COc1ccc(-c2cn3c(C)csc3n2)cc1] |
| I-132 | 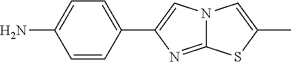 [SMILES: Cc1cn2cc(-c3ccc(N)cc3)nc2s1] |
| I-133 | 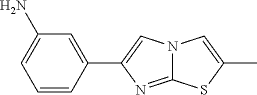 [SMILES: Cc1cn2cc(-c3cccc(N)c3)nc2s1] |
| I-134 | 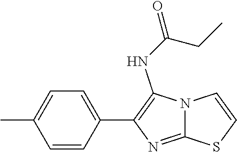 [SMILES: CCC(=O)Nc1c(-c2ccc(C)cc2)nc2sccn12] |
| I-135 | 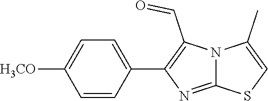 [SMILES: COc1ccc(-c2nc3scc(C)n3c2C=O)cc1] |
| I-136 | 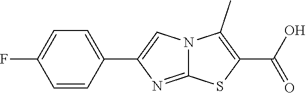 [SMILES: Cc1c(C(=O)O)sc2nc(-c3ccc(F)cc3)cn12] |
| I-137 | 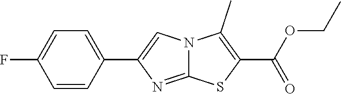 [SMILES: CCOC(=O)c1sc2nc(-c3ccc(F)cc3)cn2c1C] |
| I-138 | 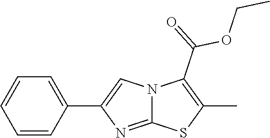 [SMILES: CCOC(=O)c1c(C)sc2nc(-c3ccccc3)cn12] |
| I-139 | 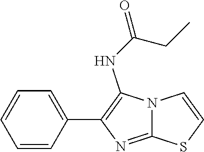 [SMILES: CCC(=O)Nc1c(-c2ccccc2)nc2sccn12] |
| I-140 | 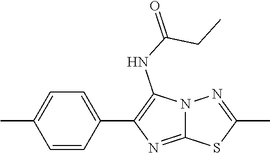 |
| I-141 | 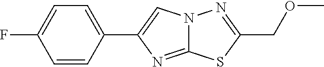 [SMILES: COCc1nn2cc(-c3ccc(F)cc3)nc2s1] |
| I-142 | 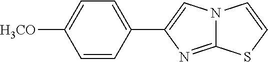 [SMILES: COc1ccc(-c2cn3ccsc3n2)cc1] |
| I-143 | 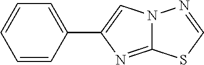 [SMILES: c1ccc(-c2cn3ncsc3n2)cc1] |
| I-144 | 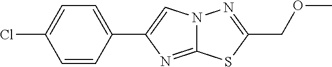 [SMILES: COCc1nn2cc(-c3ccc(Cl)cc3)nc2s1] |
| I-145 | 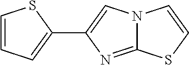 [SMILES: c1csc(-c2cn3ccsc3n2)c1] |
| I-146 | 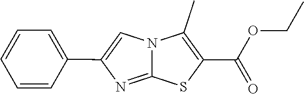 [SMILES: CCOC(=O)c1sc2nc(-c3ccccc3)cn2c1C] |
| I-147 | 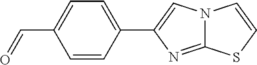 [SMILES: O=Cc1ccc(-c2cn3ccsc3n2)cc1] |
| I-148 | 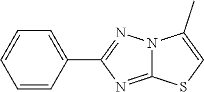 |
| I-149 | 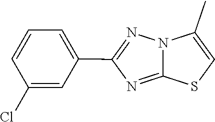 [SMILES: Cc1csc2nc(-c3cccc(Cl)c3)nn12] |
| I-150 | 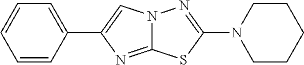 [SMILES: c1ccc(-c2cn3nc(N4CCCCC4)sc3n2)cc1] |
| I-151 | 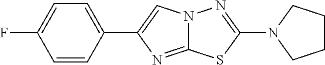 [SMILES: Fc1ccc(-c2cn3nc(N4CCCC4)sc3n2)cc1] |
| I-152 | 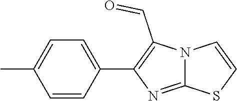 [SMILES: Cc1ccc(-c2nc3sccn3c2C=O)cc1] |
| I-153 | 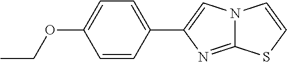 [SMILES: CCOc1ccc(-c2cn3ccsc3n2)cc1] |
| I-154 | 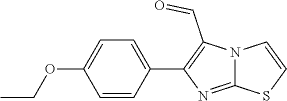 [SMILES: CCOc1ccc(-c2nc3sccn3c2C=O)cc1] |
| I-155 | 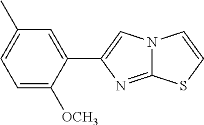 [SMILES: COc1ccc(C)cc1-c1cn2ccsc2n1] |
| I-156 | 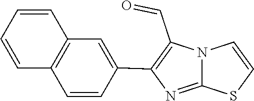 [SMILES: O=Cc1c(-c2ccc3ccccc3c2)nc2sccn12] |
| I-157 | 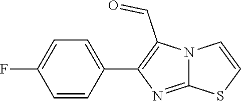 [SMILES: O=Cc1c(-c2ccc(F)cc2)nc2sccn12] |
| I-158 | 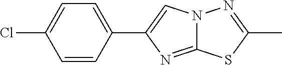 [SMILES: Cc1nn2cc(-c3ccc(Cl)cc3)nc2s1] |
| I-159 | 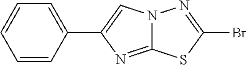 [SMILES: Brc1nn2cc(-c3ccccc3)nc2s1] |
| I-160 | 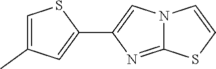 |
| I-161 | 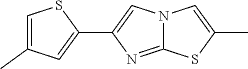 |
| I-162 | 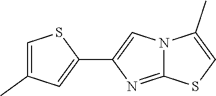 |
| | and |
| I-163 | 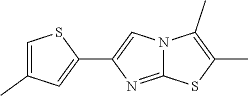 |

and/or salts and/or solvates thereof.

In some embodiments, the one or more compounds Formula (I) are selected from:

| Cpd | |
|---|---|
| No. | Structure |
| I-2 | 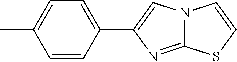 |
| I-7 | 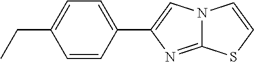 [SMILES: CCc1ccc(-c2cn3ccsc3n2)cc1] |
| I-10 | 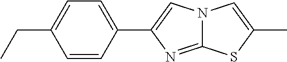 [SMILES: CCc1ccc(-c2cn3cc(C)sc3n2)cc1] |
| I-13 | 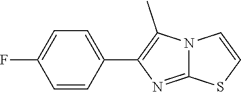 [SMILES: Cc1c(-c2ccc(F)cc2)nc2sccn12] |
| I-14 | 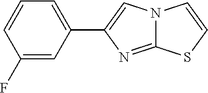 [SMILES: Fc1cccc(-c2cn3ccsc3n2)c1] |
| I-23 | 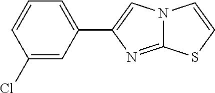 [SMILES: Clc1cccc(-c2cn3ccsc3n2)c1] |
| I-33 | 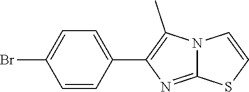 [SMILES: Cc1c(-c2ccc(Br)cc2)nc2sccn12] |
| I-34 | 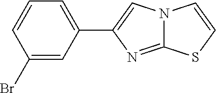 [SMILES: Brc1cccc(-c2cn3ccsc3n2)c1] |
| I-46 | 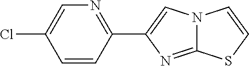 [SMILES: Clc1ccc(-c2cn3ccsc3n2)nc1] |
| I-47 | 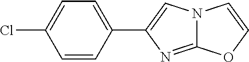 [SMILES: Clc1ccc(-c2cn3ccoc3n2)cc1] |
| I-48 | 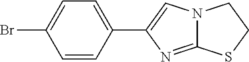 [SMILES: Brc1ccc(-c2cn3c(n2)SCC3)cc1] |
| I-49 | 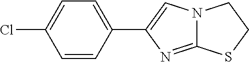 [SMILES: Clc1ccc(-c2cn3c(n2)SCC3)cc1] |
| I-52 | 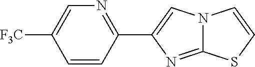 [SMILES: FC(F)(F)c1ccc(-c2cn3ccsc3n2)nc1] |
| I-63 | 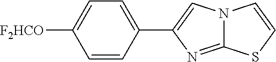 [SMILES: FC(F)Oc1ccc(-c2cn3ccsc3n2)cc1] |
| I-84 | 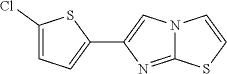 [SMILES: Clc1ccc(-c2cn3ccsc3n2)s1] |
| I-89 | 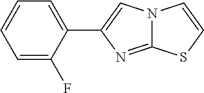 [SMILES: Fc1ccccc1-c1cn2ccsc2n1] |
| I-93 | 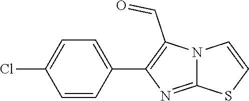 [SMILES: O=Cc1c(-c2ccc(Cl)cc2)nc2sccn12] |
| I-113 | 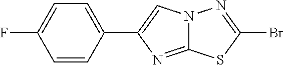 [SMILES: Fc1ccc(-c2cn3nc(Br)sc3n2)cc1] |
| I-122 | 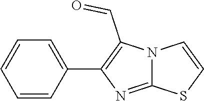 [SMILES: O=Cc1c(-c2ccccc2)nc2sccn12] |
| I-128 | 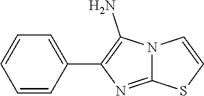 [SMILES: Nc1c(-c2ccccc2)nc2sccn12] |
| I-152 | 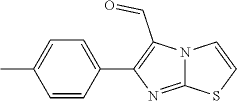 [SMILES: Cc1ccc(-c2nc3sccn3c2C=O)cc1] |
| I-154 | 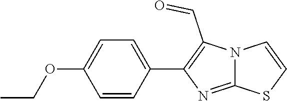 [SMILES: CCOc1ccc(-c2nc3sccn3c2C=O)cc1] |
| I-157 | 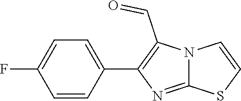 [SMILES: O=Cc1c(-c2ccc(F)cc2)nc2sccn12] |
| I-159 | 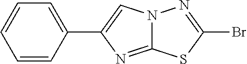 [SMILES: Brc1nn2cc(-c3ccccc3)nc2s1] |
| | and |
| I-161 | 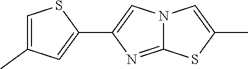 |

and/or salts and/or solvates thereof.

In some embodiments, the present application also includes a method for treating or preventing a plant nematode infection comprising administering to a plant in need thereof, an effective amount of one or more compounds of the application, and/or salts and/or solvates thereof.

The application also includes a use of one or more compounds of the application and/or salts and/or solvates thereof for treating or preventing a nematode infection. The application further includes one or more compounds of the application and/or salts and/or solvates thereof for use for treating or preventing a nematode infection.

The present application also includes a method of treating or preventing a disease, disorder or condition arising from a nematode infection comprising administering an effective amount of one or more compounds of the application and/or salts and/or solvates thereof to a subject in need thereof.

The application also includes a use of one or more compounds of the application and/or salts and/or solvates thereof for treating or preventing a disease, disorder or condition arising from a nematode infection. The application further includes one or more compounds of the application and/or salts and/or solvates thereof for use for treating or preventing a disease, disorder or condition arising from a nematode infection.

In some embodiments the salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable is for example any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt. In some embodiments, the salt is a bromide or chloride salt.

A base addition salt suitable is for example any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are agriculturally acceptable. Examples of such solvents include water (resulting solvate is called a hydrate) and ethanol and the like.

The compounds of the application useful in the present application are available from commercial sources or can be prepared using methods known in the art. For example, some of the compounds of the application can be purchased from ChemBridge Corporation, Life Chemicals and MolPort.

In some embodiments, the compounds of the application wherein X is CH are prepared as shown in Scheme 1:

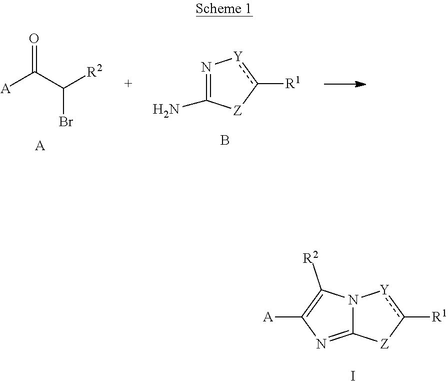

Therefore various α-bromoketones of Formula A, wherein A and R² are as defined in Formula I, are reacted with excess amounts of heteroaryl amino [symbol] compounds of Formula B, wherein [symbol] Y, Z and R¹ are as defined in Formula I, in a suitable solvent, such as a polar organic solvent, under conditions to provide the compounds of Formula 1. In some embodiments, the conditions to provide the compounds of Formula I are refluxing conditions until the disappearance of the α-bromoketone is evident by TLC, optionally followed by treatment with, for example TiCl₄, under conditions to provide the compounds of Formula 1.

In some embodiments, α-bromoketones of Formula A, wherein A is as defined in Formula I, are prepared as shown in Scheme 2:

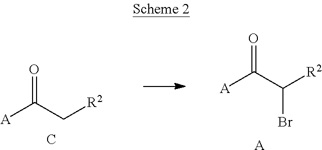

Therefore ketones of Formula C, wherein A and R² are as defined in Formula I, are brominated, for example by reaction with N-bromosuccinimide, in the presence of an acid, such as p-toluene sulfonic acid in a suitable organic solvent, by reaction with Br² in a suitable organic solvent, or by reaction with CuBr² in a suitable organic solvent to provide compounds of Formula A, wherein A and R² are as defined in Formula 1.

Compounds of Formula B, wherein [symbol] Y, Z and R¹ are as defined in Formula I, compounds of Formula C, wherein A are R² is as defined in Formula I, and compounds of Formula I, wherein X is N are either commercially available or prepared using methods known in the art.

In the above schemes, the preparation methods referenced also apply to the synthesis of compounds of Formulae I-A to I-T as defined herein as well as to all other compound Formulae covering novel compounds of the application.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

In some embodiments, the plant nematode infection is an infection of an endoparasitic nematode. In some embodiments, the nematode infection is an infection of an ectoparasitic nematode.

In some embodiments, the plant nematode infection is an infection of a nematode selected from the following genera: Meloidogyne, Heterodera, Globodera, Pratylenchus, Rotylenchulus, Hoplolaimus, Bolonolaimus, Longidorus, Paratrichodorus, Ditylenchus, Bursaphalencus, Xiphinema, Nacobbus, Aphelenchoides, Helicotylenchus, Radopholus, Hirschmanniella, Tylenchorhynchus, Trichodorus, Anguina, Criconema, Criconemella, Criconemoides, Mesocriconema, Dolichodorus, Hemicycliophora, Hemicriconemoides, Scutellonema, Tylenchulus, Subanguina, Hypsoperine, Macroposthonia, Melinius, Punctodera, and Quinisulcius.

In some embodiments, the plant nematode infection is an infection of a nematode of the genus Meloidogyne.

In some embodiments, the infection of a plant nematode of the genus Meloidogyne is an infection of a nematode belonging to the species Meloidogyne incognita.

In some embodiments, the infection of a plant nematode of the genus Meloidogyne is an infection of a nematode belonging to the species Meloidogyne chitwoodi.

In some embodiments, the infection of a plant nematode of the genus Meloidogyne is an infection of a nematode belonging to the species Meloidogyne hapla.

When used, for example, with respect to the methods of treatment, uses, compositions and kits of the application, a plant “in need thereof” is any plant, that has been diagnosed with, is suspected of having, may come in to contact with, and/or was previously treated for a nematode infection or a disease, disorder or condition arising from a nematode infection.

In some embodiments the plant is a cultivated plant. In some embodiments, the plant is an agricultural crop plant. In some embodiments, the plant includes, but is not limited to, soybeans, cotton, flax, hemp, jute, corn, tobacco, nuts, almonds, coffee, tea, pepper, grapevines, hops, wheat, barley, rye, oats, rice, maize, sorghum, apples, pears, plums, peaches, banana, plantains, cherries, strawberries, raspberries, blackberries, beans, lentils, peas, soya, oilseed rape, mustard, poppies, olives, sunflowers, coconut, castor, cocoa, ground nuts, spinach, asparagus, lettuce, cabbages, carrots, onions, tomatoes, potatoes, bell peppers, cucumbers, melons, pumpkins, sugar cane, sugar beet, fodder beet, avocado, cinnamon, camphor, oranges, tangerines, lemons, limes, grapefruit, latex plants, ornamental plants, and/or turf grasses.

In some embodiments, the disease, disorder or condition arising from a nematode infection in a plant includes, but is not limited to, stunted growth, bulb discolouration, swollen stems, root knots (or galls), root cysts, root lesions, root necrosis, toppling (or blackhead disease), and pine wilt, for example.

When used, for example, in respect to plant treatments, the compounds of the application may be delivered by several means including pre-planting, post-planting and as a feed additive, drench, or external application.

In some embodiments, the methods and uses of the application comprise applying to the plant, to the soil surrounding the plant, and/or to the seeds of the plant an effective amount of one or more compounds of the application. In some embodiments, the applying is by foliar application, for example by spraying an effective amount of one or more compounds of the application at least on to the plant leaves. In some embodiments, the applying is to the seeds of the plant, for example, as a seed coating.

In the context of treating or preventing a nematode infection or a disease, disorder or condition caused by a nematode infection in a plant, an effective amount of the one or more compounds of the application, is an amount that, for example, reduces the amount of infection by the nematode in the plant compared to the amount of infection by the nematode in the plant without administration of the one or more compounds of the application. Reducing the amount of infection may be assessed, for example, by detecting an amount of viable or living nematodes in the plant, and/or by observing or assessing the extent of a disease, disorder or condition caused by a nematode infection.

The dosage of the one or more compounds of the application in plants, varies depending on many factors such as the pharmacodynamic properties thereof, the mode of administration, the age, health and weight/mass of the plant, the nature and extent of the symptoms, the frequency of the treatment and the type of concurrent treatment, if any. One of skill in the art can determine the appropriate dosage based on the above factors. The one or more compounds of the application thereof may be administered initially in a suitable dosage that may be adjusted as required, depending on the response.

Treatment methods comprise administering to a plant one or more compounds of the application, and optionally consists of a single administration, or alternatively comprises a series of administrations. The length of the treatment period depends on a variety of factors, such as the severity of the infection, disease, disorder or condition, the age and size of the plant, the dosage of the one or more compounds of the application, the activity of one or more compounds of the application, or a combination thereof.

In some embodiments, the one or more compounds of the application are administered or used as soon as possible after exposure to the nematode. In some embodiments, the one or more compounds of the application are administered or used until treatment of the nematode infection, disease disorder or condition is achieved. For example, until complete elimination of the nematode is achieved, or until the number of nematode has been reduced to the point where the plant's defenses are no longer overwhelmed and can kill any remaining nematode.

In some embodiments, the present application includes methods of reducing the viability or fecundity or slowing the growth or development or inhibiting the infectivity of a nematode using one or more compounds of the application.

In some embodiments, the present application includes methods of reducing the viability or fecundity or slowing the growth or development or inhibiting the infectivity of a nematode using a compound of the application, the methods comprising administering an effective amount of one or more compounds of the application to a plant.

In some embodiments, the one or more compounds of the application are applied to plants at any suitable rate, the selection of which can be made by a person skilled in the art. Factors to consider include, for example, the identity of the plant, the identity of the nematode, the identity of the plant disease, disorder or condition, the severity of the nematode infection, the severity of the plant disease, disorder or condition, the age of the plant, the activity of the one or more compounds of the application and the concentration of the one or more compounds of the application, or a combination thereof.

In some embodiments, the foliage of the plant and/or the soil surrounding the plant is contacted with the one or more compounds of the application.

In some embodiments, the nematode infects plants and the one or more compounds are administered to the soil or to plants. In some embodiments, the one or more compounds are administered to soil before planting. In some embodiments, the one or more compounds are administered to soil after planting. In some embodiments, the one or more compounds are administered to soil using a drip system. In some embodiments, the one or more compounds are administered to soil using a drench system. In some embodiments, the one or more compounds are administered to plant roots or plant foliage (e.g., leaves, stems). In some embodiments the one or more compounds are tilled into the soil or administered in furrow. In some embodiments, the one or more compounds are administered to seeds. In some embodiments, the one or more compounds are applied as a seed coating.

It will also be appreciated that the effective amount of the one or more compounds of the application for the administration or use in plants may increase or decrease over the course of a particular regime. In some instances, chronic administration or use is required. In some embodiments, the one or more compounds of the application are administered or used in an amount and for a duration sufficient to control a disease, disorder or condition or eliminate the disease, disorder or condition caused by the plant nematode. In some embodiments, the one or more compounds of the application are administered or used in an amount and for a duration sufficient to control a nematode infection or eliminate the nematode infection in a plant.

The one or more compounds of the application are used either used alone or in combination with other known agents useful for treating or preventing a plant nematode infection or a disease, disorder or condition arising from a plant nematode infection. When used in combination with other agents useful for treating a plant nematode infection or a disease, disorder or condition arising from a plant nematode infection, it is an embodiment that the one or more compounds of the application are administered contemporaneously with those agents. As used herein, “contemporaneous administration” of two substances to a subject means providing each of the two substances so that they are both active in the plant at the same time.

III. Compositions of the Application

A compound of the application is suitably used on their own but will generally be administered in the form of a composition in which the one or more compounds of the application (the active ingredient) are suitably formulated in a conventional manner into compositions using one or more carriers. Accordingly, the present application also includes a composition for treating or preventing a nematode infection or a disease, a disorder, or a condition arising from a nematode infection in a plant comprising an effective amount of one or more compounds of the application, and one or more carriers. In some embodiments, the one or more compounds of the application are present in an amount that is effective to treat or prevent a nematode infection or a disease, a disorder, or a condition arising from a nematode infection in a plant.

In some embodiments, the present application includes a method of treating or preventing a nematode infection in a plant or a disease, a disorder, or a condition arising from a nematode infection in a plant comprising administering one or more compositions of the application to a plant in need thereof.

In some embodiments, the present application also includes a use of one or more compositions of the application for treating or preventing a nematode infection or a disease, a disorder, or a condition arising from a nematode infection in a plant in need thereof. The present application also includes a use of one or more compositions of the application for preparation of a medicament for treating or preventing a nematode infection or a disease, a disorder, or a condition arising from a nematode infection in a plant in need thereof. Also included is one or more compositions of the application for use to treat or prevent a nematode infection or a disease, a disorder, or a condition arising from a nematode infection in a plant in need thereof.

In some embodiments, the one or more carriers are selected from any solid or liquid carrier that is compatible with the treatments of plants.

In some embodiments, the one or more carriers is one or more agricultural excipients or one or more solvents or combinations thereof.

In some embodiments, the one or more solvents is any solvent that is compatible or suitable for the treatment of plants, such as water. In some embodiments, the solvent comprises a mixture of one or more solvents.

In some embodiments, the composition of the application is a liquid concentrate that will be diluted, for example with water, prior to use (e.g. prior to application to plants). Dilution amounts will depend, for example on the type of plant and the size of the area to be treated, and can be readily determined by a person skilled in the art. In some embodiments, the concentrate is diluted to apply or administer an effective amount of the one or more compounds of the application to the plant.

In some embodiments, the composition is a solid composition that is reconstituted or dissolved in one or more solvents, such as water, prior to use (e.g., prior to application to plants).

In some embodiments, the solid composition is reconstituted or dissolved in one or more solvents to apply or administer an effective amount of the one or more compounds of the application to the plant.

In some embodiments, depending on the mode of administration, the composition will comprise from about 0.05 wt % to about 99.95 wt % or about 0.10 wt % to about 70 wt %, of the one or more compounds of the application, and from about 1 wt % to about 99.95 wt % or about 30 wt % to about 99.90 wt % of the carrier, all percentages by weight being based on the total composition.

In some embodiments, the composition of the application is a ready to use composition and the amount of the one or more compounds of the application in the composition is about 0.001 μM to about 100 mM, 0.01 μM to about 10 mM, 0.1 μM to about 500 μM, about 1.0 μM to about 250 μM, or about 5.0 μM to about 100 μM.

In some embodiments, the one or more agricultural excipients is a surfactant, a permeation enhancer, a co-solvent, a fertilizer, a wetting agent, a sticker/spreader, a stabilizer, or an emulsifier.

For example, in some embodiments, the compositions of the application may comprise one or more aqueous surfactants. Examples of surfactants that can be used include, Span 20, Span 40, Span 80, Span 85, Tween 20, Tween 40, Tween 80, Tween 85, Triton X 100, Makon 10, Igepal CO 630, Brij 35, Brij 97, Tergitol TMN 6, Dowfax 3B2, Physan and Toximul TA 15, and mixtures thereof. In some embodiments, the surfactant is a cationic surfactant. In another embodiment of the present application, the cationic surfactant is cetyltrimethylammonium chloride.

In some embodiments, the compositions of the application may comprise a one or more permeation enhancers (e.g., cyclodextrin).

In some embodiments, the compositions of the application may comprise one or more co-solvents. Examples of co-solvents that can be used include ethyl lactate, methyl soyate/ethyl lactate co-solvent blends (e.g., Steposol), isopropanol, acetone, 1,2-propanediol, n-alkylpyrrolidones (e.g., the Agsolex series), a petroleum based-oil (e.g., aromatic 200) or a mineral oil (e.g., paraffin oil), or mixtures thereof.

In some embodiments, the compositions of the application may comprise one or more other pesticides (e.g., nematicide, insecticide or fungicide) such as an avermectin (e.g., abamectin), milbemycin, imidacloprid, aldicarb, oxamyl, fenamiphos, fosthiazate, metam sodium, etridiazole, penta-chloro-nitrobenzene (PCNB), flutolanil, metalaxyl, mefonoxam, fosetyl-al, fluensulfone, fluopyram, fluazaindolizine, iprodione, spirotetramat, and tioxazafen, or mixtures thereof. Useful fungicides include, but are not limited to, silthiofam, fludioxonil, myclobutanil, azoxystrobin, chlorothalonil, propiconazole, tebuconazole, pyraclostrobin, fluopyram and iprodione, or mixtures thereof. In some embodiments, the compositions of the application may also comprise one or more herbicides (e.g., trifloxysulfuron, glyphosate, halosulfuron) and/or other chemicals for disease control (e.g., chitosan).

In some embodiments, the compositions of the present application may comprise one or more fertilizers. In some embodiments, the fertilizer comprises primary, secondary and tertiary nutrients, for example nitrogen, phosphorous, potassium, calcium, magnesium, sulfur, zinc, manganese, iron, copper molybdenum, boron, cobalt, nickel and silicon.

In some embodiments, the compositions of the present application may comprise one or more wetting agents. In some embodiments, the wetting agent is an alcohol ethoxylate, alkylphenol ethoxylate, fatty acid ethoxylate, fatty acid ester or silicone polymer, or a mixture thereof.

In some embodiments, the compositions of the present application may comprise one or more stabilizers/emulsifiers. In some embodiments, the stabilizer/emulsifier is a polysaccharide or protein, or a mixture thereof. In another embodiment the stabilizer/emulsifier is guar gum.

In some embodiments, the compositions of the present application may comprise one or more stickers or spreaders.

In some embodiments, the compositions of the application optionally include further components. For example, inorganic bases such as an alkali metal hydroxide (e.g. potassium or sodium hydroxide), an alkali metal carbonate (e.g. potassium or sodium carbonate) or an alkali metal bicarbonate (e.g. sodium or potassium bicarbonate) can be used in combination with the amine to provide a composition with a desired pH.

In some embodiments, the compositions of the present application further include one or more additional acids (for example inorganic acids such as phosphoric acid or organic acids such as acetic acid), for example to provide a composition with a desired pH.

In some embodiments, the composition is prepared by a method comprising mixing the one or more compounds of the application, and optionally, the further components with one or more carriers under conditions to obtain the composition.

In some embodiments, the present application includes a kit for preventing and/or treating a nematode infection or a plant disease caused by a plant infection by a nematode comprising one or more compounds or compositions of the application; and instructions for administration of the one or more compounds or compositions of the application, to a plant in need thereof.

In some embodiments the instructions for administration comprise details for diluting, reconstituting or dissolving the one or more compositions of the application so that an effective amount of the one or more compounds of the application, are administered to the plant. In some embodiments the instructions for administration comprise details for preparing one or more compositions of the application, and optionally, diluting, reconstituting or dissolving the one or more compositions of the application so that an effective amount of the one or more compounds of the application, are administered to the plant.

In some embodiments, the one or more compositions of the application are applied to plants at any suitable rate, the selection of which can be made by a person skilled in the art. Factors to consider include, for example, the identity of the plant, the identity of the nematode, the identity of the plant disease, disorder or condition, the severity of the nematode infection, the severity of the plant disease, disorder or condition, the age of the plant, the concentration of the composition of the application and/or a combination thereof. For example, plants that are planted in rows (row crops) tend to use smaller volumes of water, therefore application rates for a row crop may be about 0.5 L to about 1 L of a composition diluted in about 10 L to about 80 L of water per acre. For vegetable crops application rates may be about 1 L to about 2 L of a composition in about 40 L to about 100 L of water per acre. In some embodiments, the compositions of the present application are applied 1 to 10 times, 2 to 8 times or 4 to 6 times. In some embodiments, about 0.1 L to about 2 L of a composition per acre of crop is applied one to 10 times with applications being made at least one day to at least one week apart. In all embodiments, the composition is diluted so that an effective amount, as defined above, of the one or more compounds of the application are applied to the plants.

In some embodiments, the foliage of the plant and/or the soil surrounding the plant is contacted with the one or more compositions of the application.

In some embodiments, the nematode infects plants and the one or more compositions are administered to the soil or to plants. In some embodiments, the one or more compositions are administered to soil before planting. In some embodiments, the one or more compositions are administered to soil after planting. In some embodiments, the one or more compositions are administered to soil using a drip system. In some embodiments, the one or more compositions are administered to soil using a drench system. In some embodiments, the one or more compositions are administered to plant roots or plant foliage (e.g., leaves, stems). In some embodiments the one or more compositions are tilled into the soil or administered in furrow. In some embodiments, the one or more compositions are administered to seeds.

In some embodiments, the one or more compositions are solid or powder and are administered by spreading.

In some embodiments, the methods of the application comprise administering one or more compositions of the application through one or more means selected from pre-planting, post-planting, as a feed additive, a drench and an external application.

It will also be appreciated that the effective amount of the one or more compositions of the application used for the administration or use may increase or decrease over the course of a particular regime. In some instances, chronic administration or use is required. In some embodiments, the one or more compositions of the application are administered or used in an amount and for a duration sufficient to control a disease, disorder or condition or eliminate the disease, disorder or condition caused by the plant nematode. In some embodiments, the one or more compositions of the application are administered or used in an amount and for a duration sufficient to control a nematode infection or eliminate the nematode infection in a plant.

IV. Novel Compounds

The present application also includes all novel compounds which fall within the scope of Formula I and therefore are useful in the methods and uses described herein. In some embodiments, the present application also includes a compound of Formula I-F or a salt and/or solvate thereof:

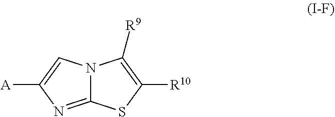

wherein:
R⁹ is H or CH₃,
R¹⁰ is H or CH₃;
A¹ is

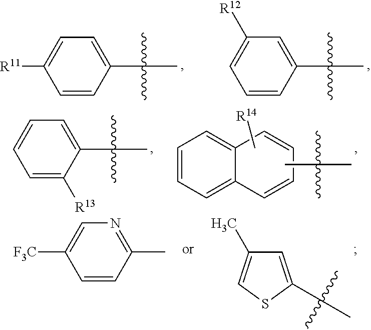

R¹¹ is C₁₋₄fluoroalkyl, OC₁₋₄fluoroalkyl, CN, CO₂C₁₋₄alkyl or C(O)C₁₋₄alkyl;
R¹² is F or I;
R¹³ is F, Cl or I; and
R¹⁴ is H or OC₁₋₄alkyl,
provided that:
when A¹ is

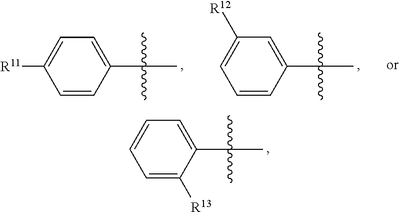

then R¹⁰ is CH₃; and
when R¹¹ is C₁₋₄fluoroalkyl or OC₁₋₄fluoroalkyl, then R⁹ is H.

In some embodiments, the compound of Formula I-F are selected from I-11, 1-17, 1-21, I-33, I-34, 1-50, 1-51, 1-52, 1-53, 1-54, 1-56, 1-58, I-73, I-75, I-81, I-82, I-83, 1-160, 1-161, 1-162 and I-163, or a salt and/or solvate thereof.

The present application also includes novel compounds 1-28, I-39, I-42, I-43, 1-67, I-70, I-71, I-76 or I-77, or a salt and/or solvate thereof.

In some embodiments, the present application includes a compound of Formula I-G, or a salt and/or solvate thereof:

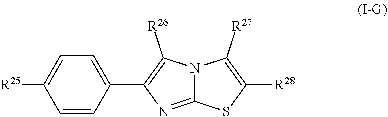

wherein:
R²⁵ is selected from F, Cl, Br, Me, OMe, CF₃, OCF₃, OCF₂H, CN and COOH;
R²⁶ is selected from H, Me and CHO; and
R²⁷ and R²⁸ are independently selected from H and Me,
with the provisos that:
(1) if R²⁵ is F or Cl or Br or Me or OMe then:
   a. R²⁶, R²⁷ and R²⁸ are not simultaneously H,
   b. R²⁶ is not Me if R²⁷ and R²⁸ are simultaneously H,
   c. R²⁷ is not Me if R²⁶ and R²⁸ are simultaneously H,
   d. R²⁸ is not Me if R²⁶ and R²⁷ are simultaneously H,
   e. R²⁶ and R²⁷ are not Me if R²⁸ is H,
   f. R²⁷ and R²⁸ are not Me if R²⁶ is H,
   g. R²⁶, R²⁷ and R²⁸ are not simultaneously Me,
   h. R²⁷ and R²⁸ are not simultaneously H if R²⁶ is CHO,
   i. R²⁷ is not Me if R²⁸ is H and R²⁶ is CHO,
   j. R²⁸ is not Me if R²⁷ is H and R²⁶ is CHO, and
   k. R²⁷ and R²⁸ are not simultaneously Me if R²⁶ is CHO;
(2) if R²⁵ is CF₃ then:
   a. R²⁶, R²⁷ and R²⁸ are not simultaneously H,
   b. R²⁷ is not Me if R²⁶ and R²⁸ are simultaneously H,
   c. R²⁷ and R²⁸ are not Me if R²⁶ is H,
   d. R²⁷ and R²⁸ are not simultaneously H if R²⁶ is CHO,
   e. R²⁷ is not Me if R²⁸ is H and R²⁶ is CHO,
   f. R²⁸ is not Me if R²⁷ is H and R²⁶ is CHO, and
   g. R²⁷ and R²⁸ are not simultaneously Me if R²⁶ is CHO;
(3) if R²⁵ is OCF₂H then:
   a. R²⁶, R²⁷ and R²⁸ are not simultaneously H,
   b. R²⁷ is not Me if R²⁶ and R²⁸ are H, and
   c. R²⁷ and R²⁸ are not simultaneously Me if R²⁶ is H;
(4) if R²⁵ is OCF₃ then:
   a. R²⁶, R²⁷ and R²⁸ are not simultaneously H,
   b. R²⁷ is not Me if R²⁶ and R²⁸ are simultaneously H,
   c. R²⁷ and R²⁸ are not simultaneously Me if R²⁶ is H, and
   d. R²⁷ and R²⁸ are not simultaneously H if R²⁶ is CHO;
(5) if R²⁵ is CN then:
   a. R²⁶, R²⁷ and R²⁸ are not simultaneously H,
   b. R²⁷ is not Me if R²⁶ and R²⁸ are simultaneously H,
   c. R²⁸ is not Me if R²⁶ and R²⁷ are simultaneously H,
   d. R²⁷ and R²⁸ are not simultaneously Me if R²⁶ is H,
   e. R²⁷ and R²⁸ are not simultaneously H if R²⁶ is CHO,
   f. R²⁷ is not Me if R²⁸ is H and R²⁶ is CHO, and
   g. R²⁸ is not Me if R²⁷ is H and R²⁶ is CHO; and
(6) if R²⁵ is COOH then R²⁶, R²⁷ and R²⁸ are not simultaneously H.

In some embodiments, the present application includes a compound of Formula I-H, or a salt and/or solvate thereof:

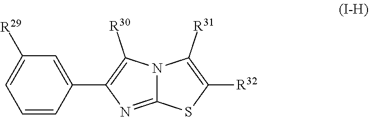

wherein:
R²⁹ is selected from F, Cl, Br, Me, OMe, CF₃, OCF₃, OCF₂H, CN and COOH;
R³⁰ is selected from H, Me and CHO; and
R³¹ and R³² are independently selected from H and Me;
with the provisos that:
(1) if R²⁹ is F then:
   a. R³⁰, R³¹ and R³² are not simultaneously H,
   b. R³⁰ is not Me if R³¹ and R³² are simultaneously H,
   c. R³¹ is not Me if R³⁰ and R³² are simultaneously H,
   d. R³⁰ and R³¹ are not simultaneously Me if R³² is H,
   e. R³¹ and R³² are not simultaneously Me if R³⁰ is H,
   f. R³⁰, R³¹ and R³² are not simultaneously Me,
   g. R³¹ and R³² are not simultaneously H if R³⁰ is CHO,
   h. R³¹ is not Me if R³² is H and R³⁰ is CHO, and
   i. R³² is not Me if R³¹ is H and R³⁰ is CHO;
(2) if R²⁹ is Cl or Br then:
   a. R³⁰, R³¹ and R³² are not simultaneously H,
   b. R³⁰ is not Me if R³¹ and R³² are simultaneously H,
   c. R³¹ is not Me if R³⁰ and R³² are simultaneously H,
   d. R³² is not Me if R³⁰ and R³¹ are simultaneously H,
   e. R³⁰ and R³¹ are not simultaneously Me if R³² is H,
   f. R³¹ and R³² are not simultaneously Me if R³⁰ is H,
   g. R³⁰, R³¹ and R³² are not simultaneously Me,
   h. R³¹ and R³² are not simultaneously H if R³⁰ is CHO,
   i. R³¹ is not Me if R³² is H and R³⁰ is CHO, and
   j. R³² is not Me if R³¹ is H and R³⁰ is CHO;
(3) if R²⁹ is Me then:
   a. R³⁰, R³¹ and R³² are not simultaneously H,
   b. R³⁰ is not Me if R³¹ and R³² are simultaneously H,
   c. R³¹ is not Me if R³⁰ and R³² are simultaneously H,
   d. R³¹ and R³² are not simultaneously Me if R³⁰ is H,
   e. R³¹ and R³² are not simultaneously H if R³⁰ is CHO,
   f. R³¹ is not Me if R³² is H and R³⁰ is CHO, and
   g. R³² is not Me if R³¹ is H and R³⁰ is CHO;
(4) if R²⁹ is OMe then:
   a. R³⁰, R³¹ and R³² are not simultaneously H,
   b. R³¹ is not Me if R³⁰ and R³² are simultaneously H,
   c. R³² is not Me if R³⁰ and R³¹ are simultaneously H,
   d. R³¹ and R³² are not simultaneously Me if R³⁰ is H,
   e. R³¹ and R³² are not simultaneously H if R³⁰ is CHO,
   f. R³¹ is not Me if R³² is H and R³⁰ is CHO,
   g. R³² is not Me if R³¹ is H and R³⁰ is CHO, and
   h. R³¹ and R³² are simultaneously Me if R³⁰ is CHO;
(5) if R²⁹ is CF₃ then:
   a. R³⁰, R³¹ and R³² are simultaneously H,
   b. R³¹ is not Me if R³⁰ and R³² are simultaneously H,
   c. R³² is not Me if R³⁰ and R³¹ are simultaneously H,
   d. R³¹ and R³² are not simultaneously Me if R³⁰ is H,
   e. R³¹ and R³² are not simultaneously be H if R³⁰ is CHO,
   f. R³¹ is not Me if R³² is H and R³⁰ is CHO, and
   g. R³² is not Me if R³¹ is H and R³⁰ is CHO;
(6) if R²⁹ is OCF₂H then:
   a. R³⁰, R³¹ and R³² are not simultaneously H,
   b. R³¹ is not Me if R³⁰ and R³² are simultaneously H, and
   c. R³¹ and R³² are not simultaneously Me if R³⁰ is H;
(7) if R²⁹ is OCF₃ then:
   a. R³⁰, R³¹ and R³² are not simultaneously H, and
   b. R³¹ and R³² are not simultaneously H if R³⁰ is CHO;
(8) if R²⁹ is CN then:
   a. R³⁰, R³¹ and R³² are not simultaneously H,
   b. R³¹ is not Me if R³⁰ and R³² are simultaneously H,
   c. R³¹ and R³² are not simultaneously Me if R³⁰ is H,
   d. R³¹ and R³² are not simultaneously H if R³⁰ is CHO,
   e. R³¹ is not Me if R³² is H and R³⁰ is CHO, and
   f. R³² is not Me if R³¹ is H and R³⁰ is CHO; and
(9) if R²⁹ is COOH then R³⁰, R³¹ and R³² are not simultaneously H.

In some embodiments, the present application includes a compound of Formula I-J, or a salt and/or solvate thereof:

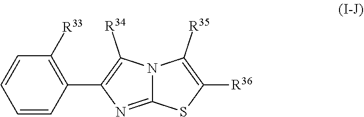

wherein:
R³³ is selected from F, Cl, Br, Me, OMe, CF₃, OCF₃, OCF₂H, CN and COOH;
R³⁴ is selected from H, Me and CHO; and
R³⁵ and R³⁶ are independently selected from H and Me;
with the provisos that:
(1) if R³³ is F then:
   a. R³⁴, R³⁵ and R³⁶ are not simultaneously H,
   b. R³⁴ is not Me if R³⁵ and R³⁶ are simultaneously H,
   c. R³⁵ is not Me if R³⁴ and R³⁶ are simultaneously H,
   d. R³⁴ and R³⁵ are not simultaneously Me if R³⁶ is H,
   e. R³⁵ and R³⁶ are not simultaneously Me if R³⁴ is H,
   f. R³⁴, R³⁵ and R³⁶ are not simultaneously Me,
   g. R³⁵ and R³⁶ are not simultaneously H if R³⁴ is CHO,
   h. R³⁵ is not Me if R³⁶ is H and R³⁴ is CHO, and
   i. R³⁶ is not Me if R³⁵ is H and R³⁴ is CHO;
(2) if R³³ is Cl or Br then:
   a. R³⁴, R³⁵ and R³⁶ are not simultaneously H,
   b. R³⁵ is not Me if R³⁴ and R³⁶ are simultaneously H,
   c. R³⁶ is not Me if R³⁴ and R³⁵ are simultaneously H,
   d. R³⁵ and R³⁶ are not simultaneously Me if R³⁴ is H,
   e. R³⁵ and R³⁶ are not simultaneously H if R³⁴ is CHO,
   f. R³⁵ is not Me if R³⁶ is H and R³⁴ is CHO, and
   g. R³⁶ is not Me if R³⁵ is H and R³⁴ is CHO;
(3) if R³³ is Me or CF₃ then:
   a. R³⁴, R³⁵ and R³⁶ are not simultaneously H,
   b. R³⁵ is not Me if R³⁴ and R³⁶ are simultaneously H,
   c. R³⁵ and R³⁶ are not simultaneously Me if R³⁴ is H,
   d. R³⁵ and R³⁶ are not simultaneously H if R³⁴ is CHO,
   e. R³⁵ is not Me if R³⁶ is H and R³⁴ is CHO, and
   f. R³⁶ is not Me if R³⁵ is H and R³⁴ is CHO;
(4) if R³³ is OMe then:
   a. R³⁴, R³⁵ and R³⁶ are not simultaneously H,
   b. R³⁵ is not Me if R³⁴ and R³⁶ are simultaneously H,
   c. R³⁶ is not Me if R³⁴ and R³⁵ are simultaneously H,
   d. R³⁵ and R³⁶ are not simultaneously Me if R³⁴ is H,
   e. R³⁵ and R³⁶ are not simultaneously H if R³⁴ is CHO,
   f. R³⁵ is not Me if R³⁶ is H and R³⁴ is CHO,
   g. R³⁶ is not Me if R³⁵ is H and R³⁴ is CHO, and
   h. R³⁵ and R³⁶ are not simultaneously Me if R³⁴ is CHO;
(5) if R³³ is CN then R³⁴, R³⁵ and R³⁶ are not simultaneously H;
(6) if R³³ is COOH then:
   a. R³⁴, R³⁵ and R³⁶ are not simultaneously H, and
   b. R³⁵ is not Me if R³⁴ and R³⁶ are simultaneously H;
(7) if R³³ is OCF₂H then:
   a. R³⁴, R³⁵ and R³⁶ are not simultaneously H,
   b. R³⁵ is not Me if R³⁴ and R³⁶ are simultaneously H, and
   c. R³⁵ and R³⁶ are not simultaneously Me if R³⁴ is H; and
(8) if R³³ is OCF₃ then:
   a. R³⁴, R³⁵ and R³⁶ are not simultaneously H, and
   b. R³⁵ and R³⁶ are not simultaneously H if R³⁴ is CHO.

In some embodiments, the present application includes a compound of Formula I-K, or a salt and/or solvate thereof:

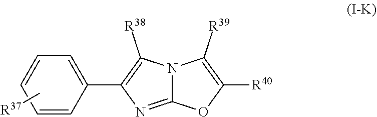

wherein:
R³⁷ represents one or more substituents each of which is independently selected from F, Cl, Br, Me, Et, Pr, iPr, OMe, OEt, OPr, OiPr, CF₃, OCF₃, OCF₂H, CN and COOH;
R³⁸ is selected from H, Me and CHO; and
R³⁹ and R⁴⁰ are independently selected from H and Me;
with the provisos that:
(1) if R³⁷ is 4-OMe then:
   a. R³⁸, R³⁹ and R₄ are not simultaneously H,
   b. R³⁹ is not Me if R³⁸ and R⁴⁰ are simultaneously H, and
   c. R³⁹ and R⁴⁰ are not simultaneously Me if R³⁸ is H;
(2) if R³⁷ is 4-F then:
   a. R³⁸, R³⁹ and R⁴⁰ are not simultaneously H,
   b. R⁴⁰ is not Me if R³⁸ and R³⁹ are simultaneously H, and
   c. R³⁹ and R⁴⁰ are not simultaneously H if R³⁸ is CHO;
(3) if R³⁷ is 3-F or 3,4-diF or 2,3,4-triF or 2,4,5-triF or 2-Cl or 2-Me or 3-OMe or 3-CF₃ or 2-Cl,4-F or 3-OMe,4-Cl or 3-OMe,4-F then:
   a. R³⁸, R³⁹ and R⁴⁰ are not simultaneously H;
(4) if R³⁷ is 2,4-diF or 2,5-diCl then:
   a. R³⁸, R³⁹ and R⁴⁰ are not simultaneously H, and
   b. R³⁹ and R⁴⁰ are not simultaneously H if R³⁸ is CHO;
(5) if R³⁷ is 4-Cl then:
   a. R³⁸, R³⁹ and R⁴⁰ are not simultaneously H,
   b. R³⁹ is not Me if R³⁸ and R⁴⁰ are simultaneously H,
   c. R³⁹ and R⁴⁰ are not simultaneously Me if R³⁸ is H, and
   d. R³⁹ and R⁴⁰ are not simultaneously H if R³⁸ is CHO;
(6) if R³⁷ is 4-Br then:
   a. R³⁹ is not Me if R³⁸ and R⁴⁰ are simultaneously H, and
   b. R³⁹ and R⁴⁰ are not simultaneously Me if R³⁸ is H; and
(7) if R³⁷ is 3-Me then:
   a. R³⁸, R³⁹ and R⁴⁰ are not simultaneously H, and
   b. R³⁹ and R⁴⁰ are not simultaneously Me if R³⁸ is H.

In some embodiments, the present application includes a compound of Formula I-L, or a salt and/or solvate thereof:

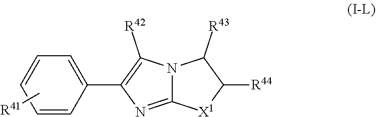

wherein:
X¹ is O or S;
R⁴¹ represents one or more optional substituents each of which is independently selected from F, Cl, Br, Me, Et, Pr, iPr, OMe, OEt, OPr, OiPr,
CF₃, OCF₃, OCF₂H, CN and COOH;
R⁴² is selected from H, Me and CHO; and
R⁴³ and R⁴⁴ is selected from H and Me;
with the provisos that:
(1) if X¹=S and the phenyl ring is unsubstituted then:
   a. R⁴², R⁴³ and R⁴⁴ are not simultaneously H,
   b. R⁴² is not Me if R⁴³ and R⁴⁴ are simultaneously H,
   c. R⁴³ is not Me if R⁴² and R⁴⁴ are simultaneously H,
   d. R⁴³ and R⁴⁴ are not simultaneously H if R⁴² is CHO,
   e. R⁴³ is not Me if R⁴² is CHO and R⁴⁴ is H,
   f. R44 is not Me if R⁴² is CHO and R⁴³ is H;
(2) if X¹=S and R⁴¹ is 2-F or 3-Cl or 2-Br or 3-Br or 2-CN or 3-CN or 4-CN or 2-COOH or 3-000H or 4-COOH then:
   a. R⁴², R⁴³ and R⁴⁴ are not simultaneously H;
(3) if X¹=S and R⁴¹ is 4-F or 4-Cl or 2,4-diCl or 3,4-diCl or 2,3,4-triCl or 4-Br or 4-Me or 4-Et or 4-iPr or 2-OMe or 3-OMe or 4-OMe or 2,4-OMe or 2,5-OMe or 4-OEt or 3-CF₃ then:
   a. R⁴², R⁴³ and R⁴⁴ are not simultaneously H,
   b. R⁴³ and R⁴⁴ are not simultaneously H if R⁴² is CHO;
(4) if X¹=S and R⁴¹ is 2,5-diF or 3,4-diF or 2-Me or 2,4-diMe or 3,4-diMe or 2,4,6-triMe or 2,3,5,6-tetraMe or 4-Pr or 3,4-diOMe or 3,4-diOEt or 4-OPr or 4-OiPr or 3-Me,4-F or 3-Me,4-Cl or 3-Me,4-OMe or 2-OMe,5-Me or 2-OMe,5-iPr or 2-OEt,5-Me or 2-Me,4-OMe,6-Me or 2-OMe,3-Me,5-Me then:
   a. R⁴³ and R⁴⁴ are not simultaneously H if R⁴² is CHO;
(5) if X¹=0 and the phenyl ring is unsubstituted then:
   a. R⁴², R⁴³ and R⁴⁴ are not simultaneously H,
   b. R⁴³ and R⁴⁴ are not simultaneously H if R⁴² is CHO;
(6) if X¹=0 and R⁴¹ is 2,4-diF or 4-CN or 4-COOH then:
   a. R⁴², R⁴³ and R⁴⁴ are not simultaneously H;
(7) if X¹=0 and R⁴¹ is 2-Me or 3-Me or 4-Me then:
   a. R⁴³ and R⁴⁴ are not simultaneously H if R⁴² is CHO.

In some embodiments, the present application includes a compound of Formula I-M, or a salt and/or solvate thereof:

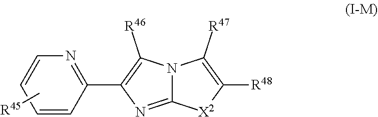

wherein:
X² is O or S;
R⁴⁵ represents one or more optional substituents each of which is independently selected from F, Cl, Br, Me, Et, Pr, iPr, OMe, OEt, OPr, OiPr,
CF₃, OCF₃, OCF₂H, CN and COOH;
R⁴⁶ is selected from H, Me and CHO; and
R⁴⁷ and R⁴⁸ is selected from H and Me;
with the provisos that:
(1) if X²=S and the pyridine ring is unsubstituted then:
   a. R⁴⁶, R⁴⁷ and R⁴⁸ are not simultaneously H,
   b. R⁴⁷ is not Me if R⁴⁶ and R⁴⁸ are simultaneously H,
   c. R⁴⁸ is not Me if R⁴⁶ and R⁴⁷ are simultaneously H,
   d. R⁴⁷ and R⁴⁸ are not simultaneously Me if R⁴⁶ is H,
   e. R⁴⁷ is not Me if R⁴⁶ is CHO and R⁴⁸ is H,
   f. R⁴⁸ is not Me if R⁴⁶ is CHO and R⁴⁷ is H,
   g. R⁴⁷ and R⁴⁸ are not simultaneously Me if R⁴⁶ is CHO, and
   h. R⁴⁷ and R⁴⁸ are not simultaneously H if R⁴⁶ is CHO;
(2) if X²=S and R⁴⁵ is 5-F then:
   a. R⁴⁶, R⁴⁷ and R⁴⁸ are not simultaneously H,
   b. R⁴⁶ is not Me if R⁴⁷ and R⁴⁸ are simultaneously H,
   c. R⁴⁷ is not Me if R⁴⁶ and R⁴⁸ are simultaneously H,
   d. R⁴⁶ and R⁴⁷ are not simultaneously Me if R⁴⁸ is H,
   e. R⁴⁷ and R⁴⁸ are not simultaneously Me if R⁴⁶ is H, and
   f. R⁴⁶, R⁴⁷ and R⁴⁸ are not simultaneously Me;
(3) if X²=S and R⁴⁵ is 5-Cl or 5-Br then:
   a. R⁴⁶, R⁴⁷ and R⁴⁸ are not simultaneously H, and
   b. R⁴⁷ is not Me if R⁴⁶ and R⁴⁸ are H; and
(4) if X²=S and R⁴⁵ is 6-COOH then R⁴⁶, R⁴⁷ and R⁴⁸ are not simultaneously H.

In some embodiments, the present application includes a compound of Formula I-N, or a salt and/or solvate thereof:

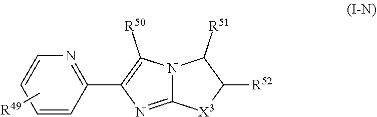

wherein:
X³ is O or S;
R⁴⁹ represents one or more optional substituents each of which is independently selected from F, Cl, Br, Me, Et, Pr, iPr, OMe, OEt, OPr, OiPr,
CF₃, OCF₃, OCF₂H, CN and COOH;
R⁵⁰ is selected from H, Me and CHO; and
R⁵¹ and R⁵² are independently selected from H and Me;
with the provisos that:
(1) if X³=S and the pyridine ring is unsubstituted then:
   a. R⁵⁰, R⁵¹ and R⁵² are not simultaneously H, and
   b. R⁵⁰ is not CHO if R⁵¹ and R⁵⁰ are simultaneously H;
(2) if X³=0 and R⁴⁹ is 6-Me then R⁵⁰ R⁵¹ and R⁵² are not simultaneously H; and
(3) if X³=0 and R⁴⁹ is 6-CN or 6-COOH then R⁵⁰ is not Me if R⁵¹ and R⁵² are simultaneously H.

In some embodiments, the present application includes a compound of Formula 1-0, or a salt and/or solvate thereof:

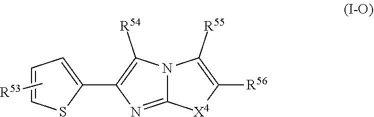

wherein:
X⁴ is O or S;
R⁵³ represents one or more optional substituents each of which is independently selected from F, Cl, Br, Me, Et, Pr, iPr, OMe, OEt, OPr, OiPr, CF₃, OCF₃, OCF₂H, CN and COOH;
R⁵⁴ is selected from H, Me and CHO; and
R⁵⁵ and R⁵⁶ are independently selected from H and Me;
with the provisos that:
(1) if X⁴=S and the thiophene ring is unsubstituted then:
   a. R⁵⁴, R⁵⁵ and R⁵⁶ are not simultaneously H,
   b. R⁵⁴ is not Me if R⁵⁵ and R⁵⁶ are simultaneously H,
   c. R⁵⁵ is not Me if R⁵⁴ and R⁵⁶ are simultaneously H,
   d. R⁵⁶ is not Me if R⁵⁴ and R⁵⁵ are simultaneously H,
   e. R⁵⁴ and R⁵⁵ are not simultaneously Me if R⁵⁶ is H,
   f. R⁵⁵ and R⁵⁶ are not simultaneously Me if R⁵⁴ is H,
   g. R⁵⁵ is not Me if R⁵⁴ is CHO and R⁵⁶ is H,
   h. R⁵⁶ is not Me if R⁵⁴ is CHO and R⁵⁵ is H,
   i. R⁵⁵ and R⁵⁶ are not simultaneously H if R⁵⁴ is CHO,
   j. R⁵⁵ and R⁵⁶ are not simultaneously Me if R⁵⁴ is CHO, and
   k. R⁵⁴, R⁵⁵ and R⁵⁶ are not simultaneously Me;
(2) if X⁴=S and R⁵³ is 5-Cl then:
   a. R⁵⁴, R⁵⁵ and R⁵⁶ are not simultaneously H,
   b. R⁵⁶ is not Me if R⁵⁴ and R⁵⁵ are simultaneously H,
   c. R⁵⁵ is not Me if R⁵⁴ and R⁵⁶ are simultaneously H,
   d. R⁵⁵ and R⁵⁶ are not simultaneously Me if R⁵⁴ is H,
   e. R⁵⁵ and R⁵⁶ are not simultaneously H if R⁵⁴ is CHO, and
   f. R⁵⁵ is not Me if R⁵⁴ is CHO and R⁵⁶ is H;
(3) if X⁴=S and R⁵³ is 5-Br or 5-Me or 5-Et then:
   a. R⁵⁴, R⁵⁵ and R⁵⁶ are not simultaneously H,
   b. R⁵⁵ is not Me if R⁵⁴ and R⁵⁶ are simultaneously H,
   c. R⁵⁵ and R⁵⁶ are not simultaneously Me if R⁵⁴ is H,
   d. R⁵⁵ is not Me if R⁵⁴ is CHO and R⁵⁶ is H, and
   e. R⁵⁵ and R⁵⁶ are not simultaneously H if R⁵⁴ is CHO; and
(4) if X⁴=S and R⁵³ is 4-Br or 3-Me then:
   a. R⁵⁴, R⁵⁵ and R⁵⁶ are not simultaneously H,
   b. R⁵⁵ is not Me if R⁵³ and R⁵⁶ are simultaneously H, and
   c. R⁵⁵ and R⁵⁶ are not simultaneously Me if R⁵⁴ is H.

In some embodiments, the present application includes a compound of Formula I-P, or a salt and/or solvate thereof:

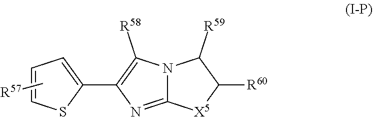

wherein:
X⁵ is O or S;
R⁵⁷ represents one or more optional substituents each of which is independently selected from F, Cl, Br, Me, Et, Pr, iPr, OMe, OEt, OPr, OiPr, CF₃, OCF₃, OCF₂H, CN and COOH;
R⁵⁸ is selected from H, Me and CHO; and
R⁵⁹ and R⁶⁰ are independently selected from H and Me;
with the provisos that:
(1) if X⁵=S and the thiophene ring is unsubstituted then:
   a. R⁵⁸, R⁵⁹ and R⁶⁰ are not simultaneously H, and
   b. R⁵⁸ and R⁶⁰ are not simultaneously H if R2 is CHO;
(2) if X⁵=S and R⁵⁷ is 4-Me or 5-Me then R⁵⁹ and R⁶⁰ are not simultaneously H if R⁵⁸ is CHO; and
(3) if X⁵=O and R⁵⁷ is 4-Me or 5-Me then R⁵⁹ and R⁶⁰ are not simultaneously H if R⁵⁸ is CHO.

In some embodiments, the present application includes a compound of Formula 1-Q, or a salt and/or solvate thereof:

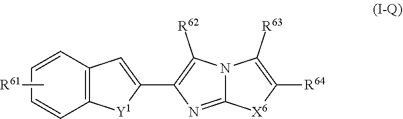

wherein:
X⁶ is O or S;
Y¹ is O or S;
R⁶¹ represents one or more optional substituents each of which is independently selected from F, Cl, Br, Me, Et, Pr, iPr, OMe, OEt, OPr, OiPr, CF₃, OCF₃, OCF₂H, CN and COOH;
R⁶² is selected from H, Me and CHO;
R⁶³ and R⁶⁴ are independently selected from H and Me;
with the provisos that:
(1) if X⁶ and Y¹ are S and the benzothiophene ring is unsubstituted or R⁶¹ is 3-Me then:
   a. R⁶², R⁶³ and R⁶⁴ are not simultaneously H,
   b. R⁶³ is not Me if R⁶² and R⁶⁴ are simultaneously H,
   c. R⁶³ and R⁶⁴ are not simultaneously Me if R⁶² is H;
(2) if X⁶ is S and Y¹ is O and the benzofuran ring is unsubstituted or R⁶¹ is 5-Br or R⁶¹ is 3-Et then:
   a. R⁶², R⁶³ and R⁶⁴ are not simultaneously H,
   b. R⁶³ is not Me if R⁶² and R⁶⁴ are simultaneously H,
   c. R⁶³ and R⁶⁴ are not simultaneously Me if R⁶² is H;
(3) if X⁶ is S and Y¹ is O and R⁶¹ is 5-Cl then R⁶³ is not Me if R⁶² and R⁶⁴ are H.

In some embodiments, the present application includes a compound of Formula I-R, or a salt and/or solvate thereof:

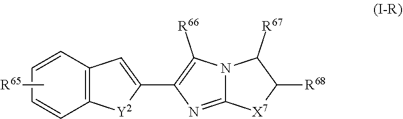

wherein:
X⁷ is O or S;
Y² is O or S;
R⁶⁵ represents one or more optional substituents each of which is independently selected from F, Cl, Br, Me, Et, Pr, iPr, OMe, OEt, OPr, OiPr, CF₃, OCF₃, OCF₂H, CN and COOH;
R⁶⁶ is selected from H, Me and CHO; and
R⁶⁷ and R⁶⁸ are independently selected from H and Me;
with the proviso that:
if X⁷ is S and Y² is O and the benzofuran ring is unsubstituted then R⁶⁵, R⁶⁶ and R67 are not simultaneously H.

In some embodiments, the present application includes a compound of Formula 1-S, or a salt and/or solvate thereof:

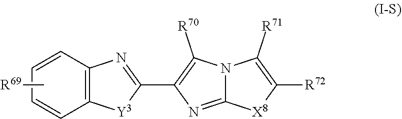

wherein:
X⁸ is O or S;
Y³ is O or S;
R⁶⁹ represents one or more optional substituents each of which is independently selected from F, Cl, Br, Me, Et, Pr, iPr, OMe, OEt, OPr, OiPr, CF₃, OCF₃, OCF₂H, CN and COOH;
R⁷⁰ is selected from H, Me and CHO; and
R⁷¹ and R⁷² are independently selected from H and Me;
with the proviso that:
(1) if X⁸ and Y³ are S and the benzothiazole ring is unsubstituted then:
   a. R⁷⁰, R⁷¹ and R⁷² are not simultaneously H,
   b. R⁷¹ is not Me if R⁷⁰ and R⁷² are simultaneously H,
   c. R⁷¹ and R⁷² are not simultaneously Me if R⁷⁰ is H.

In some embodiments, the present application includes a compound of Formula I-T, or a salt and/or solvate thereof:

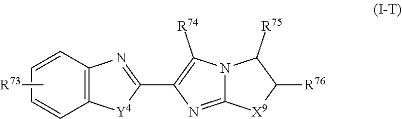

wherein:
X⁹ is O or S;
Y⁴ is O or S;
R⁷³ represents one or more optional substituents each of which is independently selected from F, Cl, Br, Me, Et, Pr, iPr, OMe, OEt, OPr, OiPr, CF₃, OCF₃, OCF₂H, CN and COOH;
R⁷⁴ is selected from H, Me and CHO;
R⁷⁵ and R⁷⁶ are independently selected from H and Me;
with the proviso that if X⁹ and Y⁴ are simultaneously S and the benzothiazole ring is unsubstituted then R⁷⁴, R⁷⁵ and R⁷⁶ are not simultaneously H.

The present application also includes the following novel compounds:
(1) A compound of one of the following formulae, or a salt and/or solvate thereof:

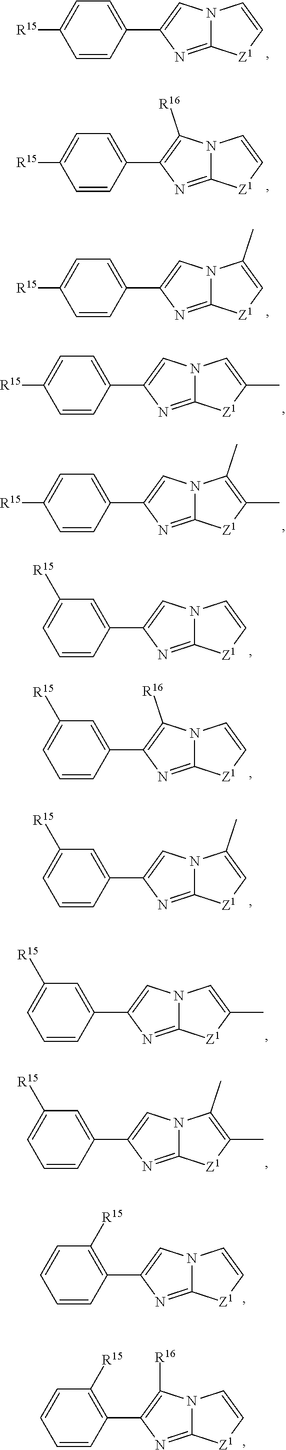

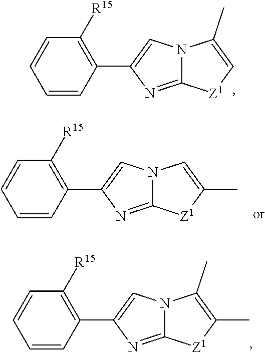

wherein
R¹⁵ is H, CH₃, CH₂CH₃, CH₂CH₂CH₃, F, CI, CN, CF₃, OCF₃, OCHF₂, C(O)OH, C(O)H, C(O)OCH₃, C(O)OCH₂CH₃, C(O)OCH₂CH₂CH₃, OCH₃, OCH₂CH₃, OCH₂CH₂CH₃, C(O)CH₃, C(O)CH₂CH₃ or C(O)CH₂CH₂CH₃;
R¹⁶ is CH₃ or C(O)H; and
Z¹ is O or S, and
provided that the compound is not:

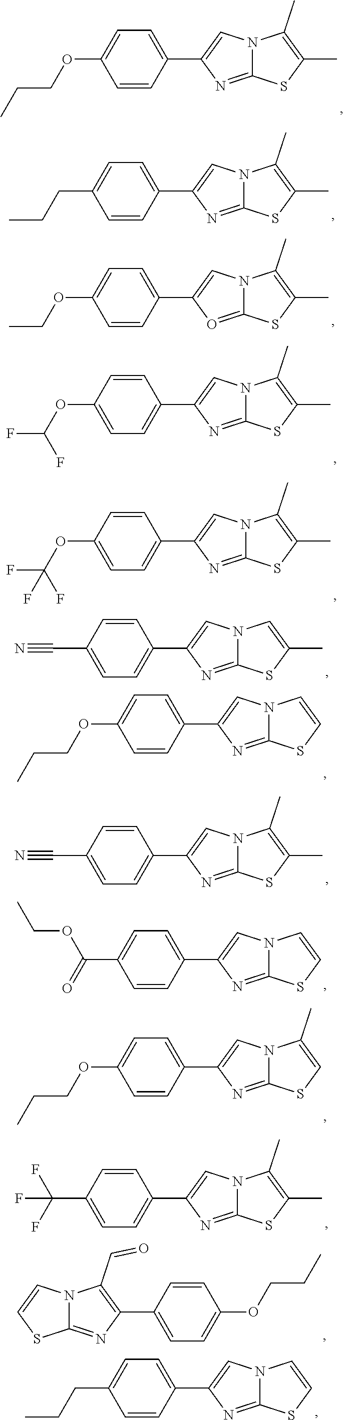

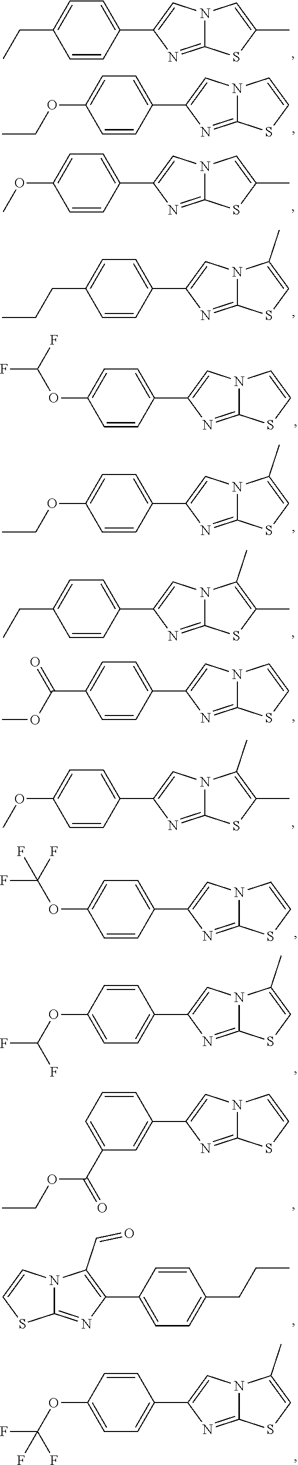

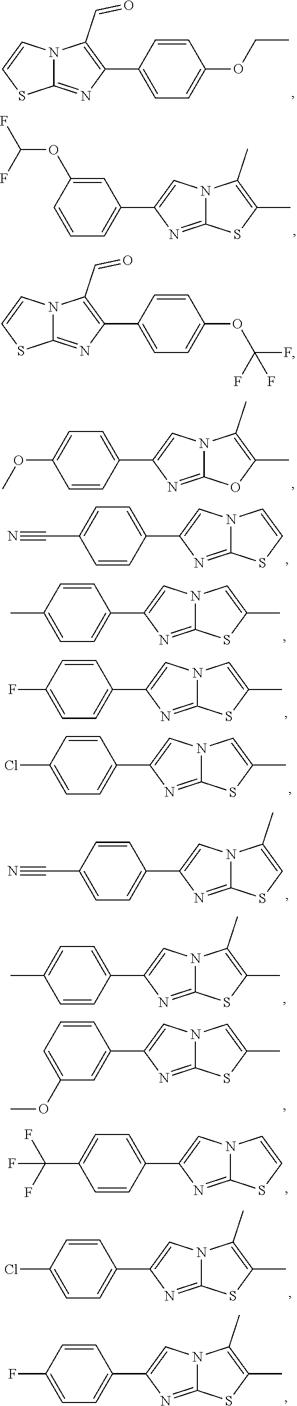

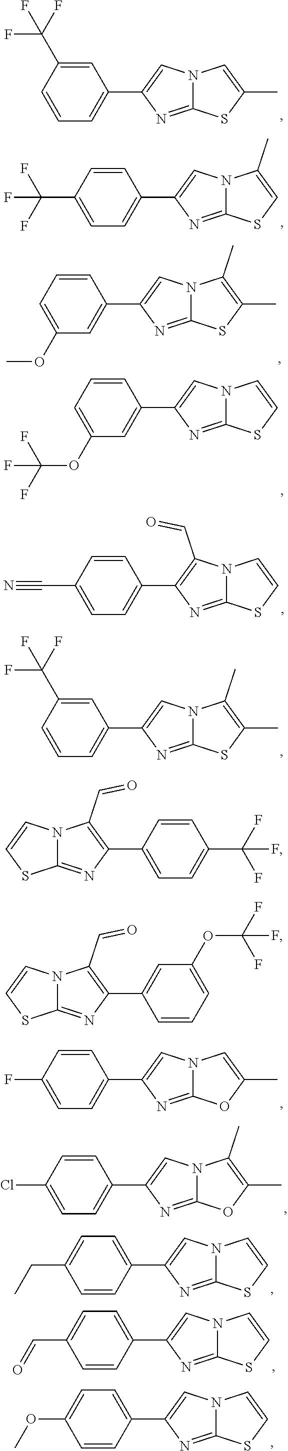

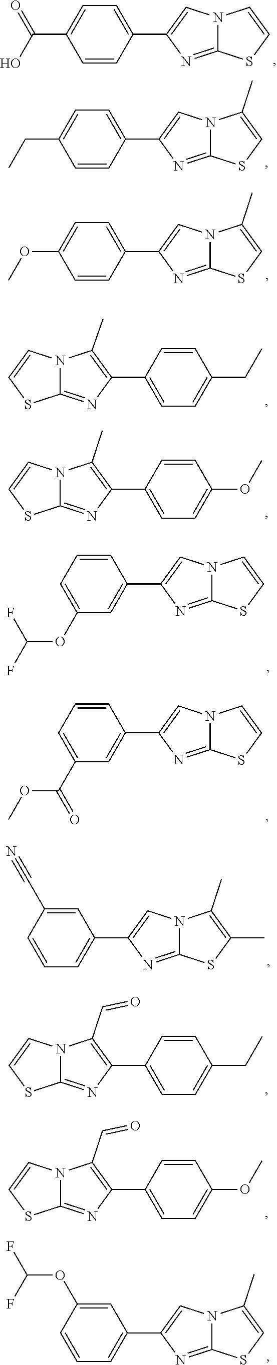

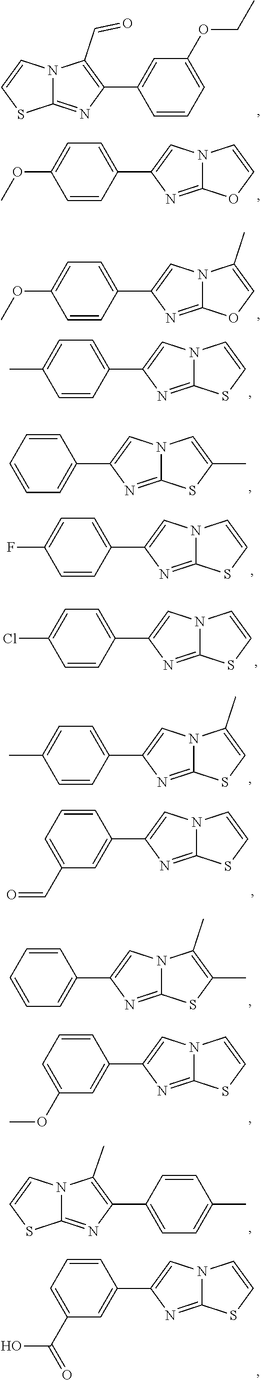

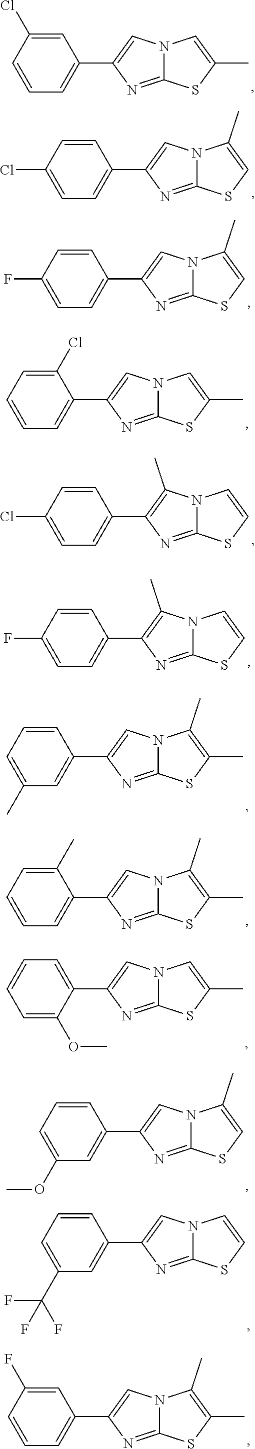

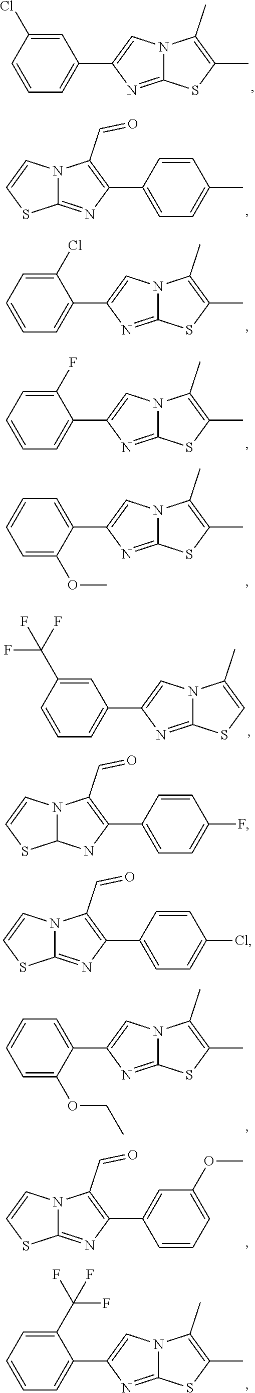

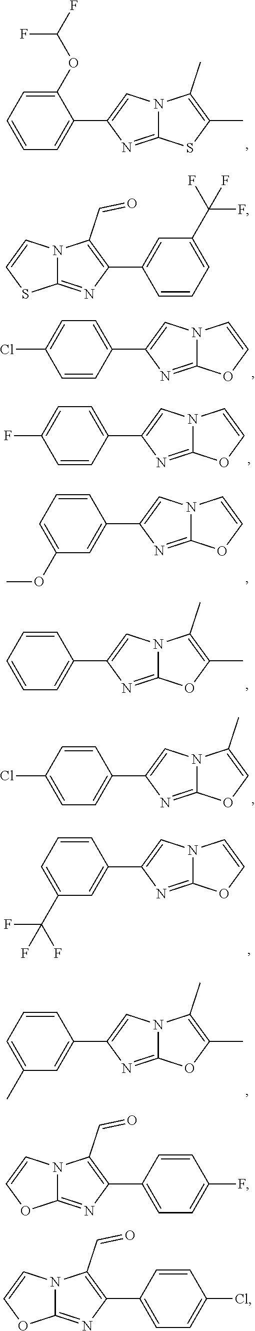

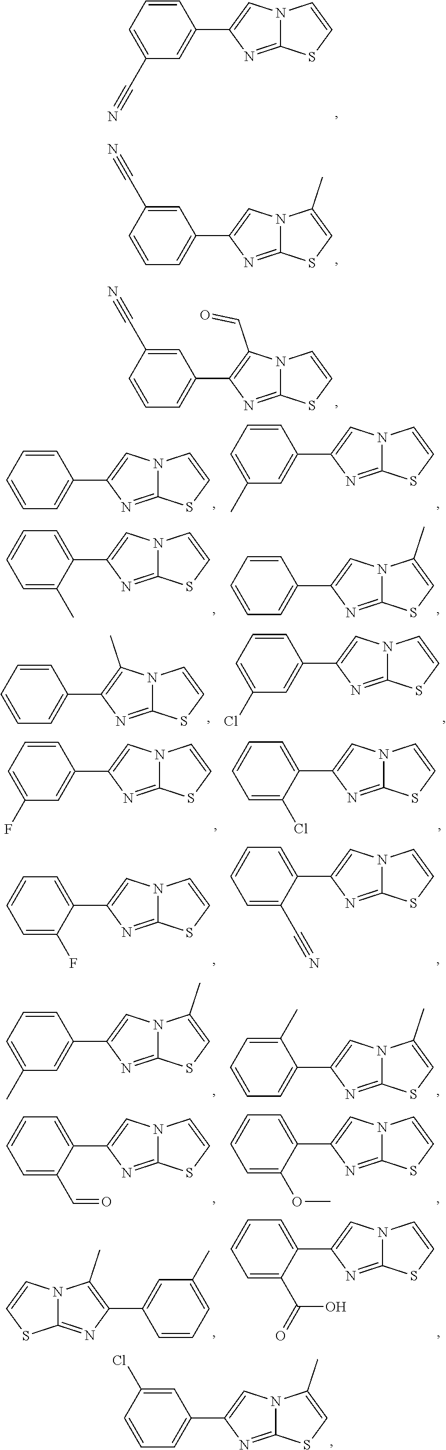

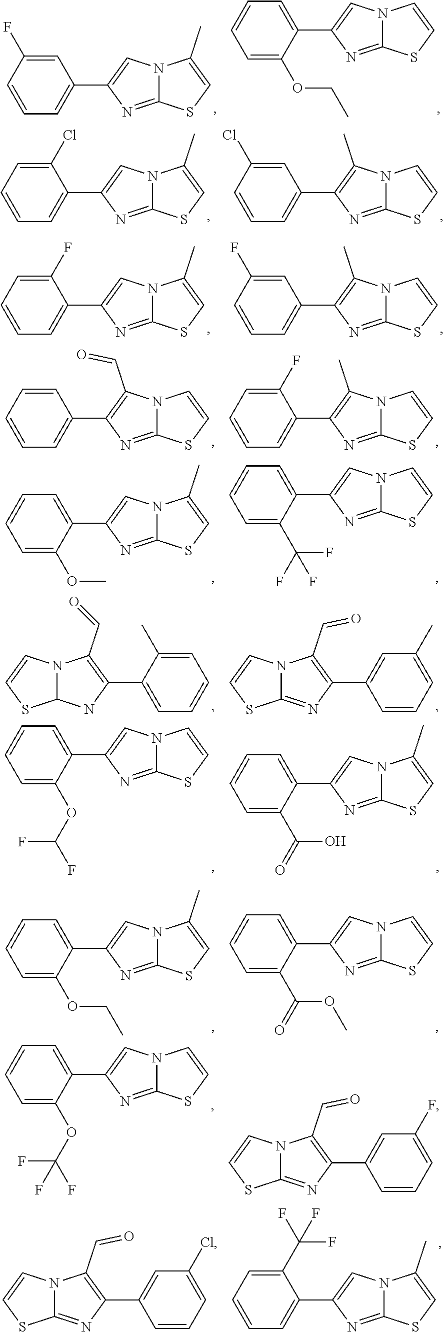

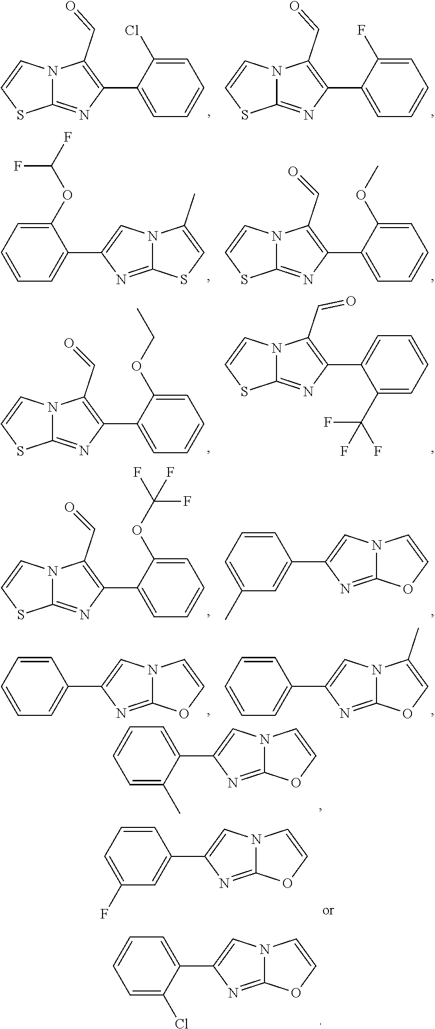

(2) A compound of one of the following formulae, or a salt and/or solvate thereof:

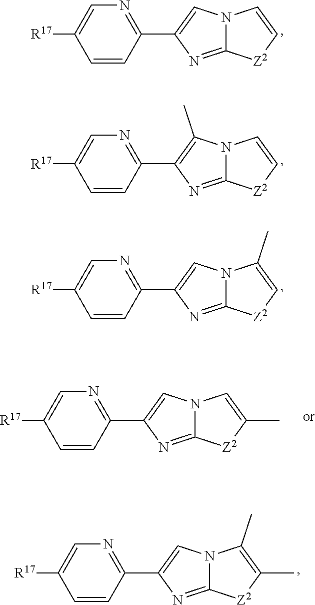

wherein:
R¹⁷ is H, CH₃, CH₂CH₃, CH₂CH₂CH₃, F, Cl, CN, CF₃, OCF₃, OCHF₂, C(O)OH, C(O)H, C(O)OCH₃, C(O)OCH₂CH₃, C(O)OCH₂CH₂CH₃, OCH₃, OCH₂CH₃, OCH₂CH₂CH₃, C(O)CH₃, C(O)CH₂CH₃ or C(O)CH₂CH₂CH₃; and
Z² is O or S, and
provided that the compound is not:

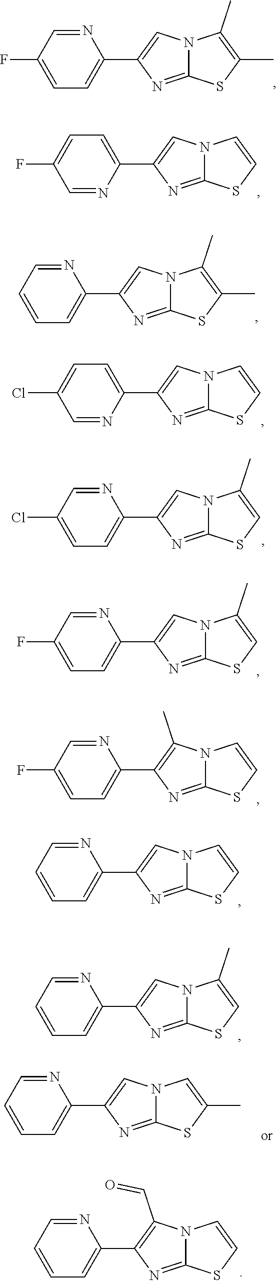

(3) A compound of one of the following formulae, or a salt and/or solvate thereof:

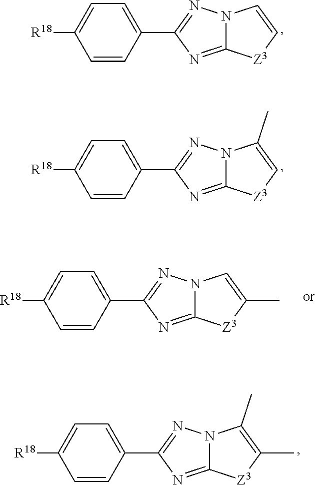

wherein:
R¹⁸ is H, CH₃, CH₂CH₃, CH₂CH₂CH₃, F, Cl, CN, CF₃, OCF₃, OCHF₂, C(O)OH, C(O)H, C(O)OCH₃, C(O)OCH₂CH₃, C(O)OCH₂CH₂CH₃, OCH₃, OCH₂CH₃, OCH₂CH₂CH₃, C(O)CH₃, C(O)CH₂CH₃ or C(O)CH₂CH₂CH₃; and
Z³ is O or S, and
provided that the compound is not:

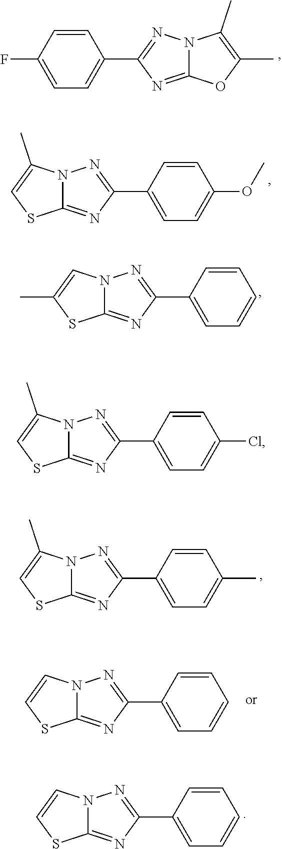

(4) A compound of one of the following formulae, or a salt and/or solvate thereof:

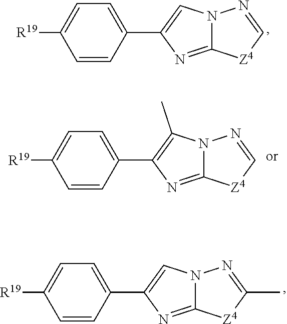

wherein:
R¹⁹ is H, CH₃, CH₂CH₃, CH₂CH₂CH₃, F, Cl, CN, CF₃, OCF₃, OCHF₂, C(O)OH, C(O)H, C(O)OCH₃, C(O)OCH₂CH₃, C(O)OCH₂CH₂CH₃, OCH₃, OCH₂CH₃, OCH₂CH₂CH₃, C(O)CH₃, C(O)CH₂CH₃ or C(O)CH₂CH₂CH₃; and
Z⁴ is O or S, and
provided that the compound is not:

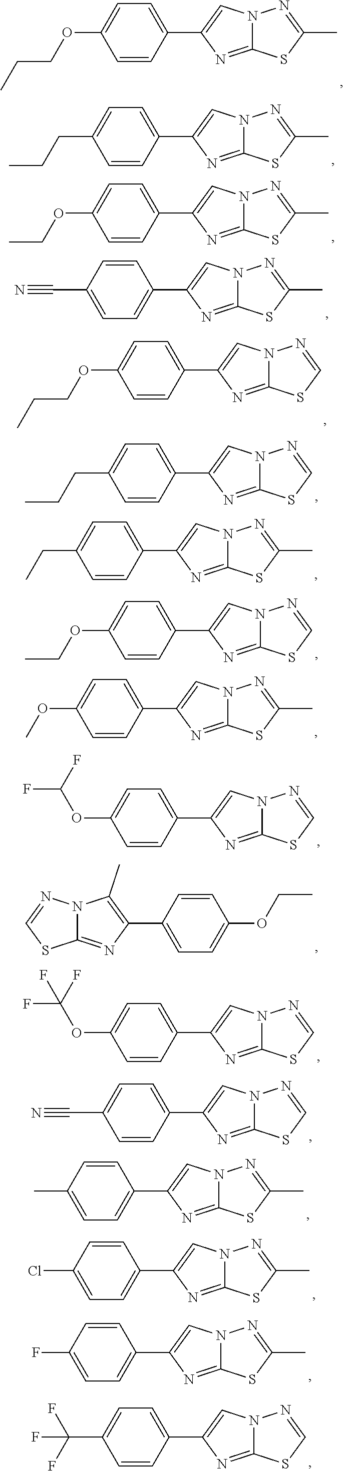

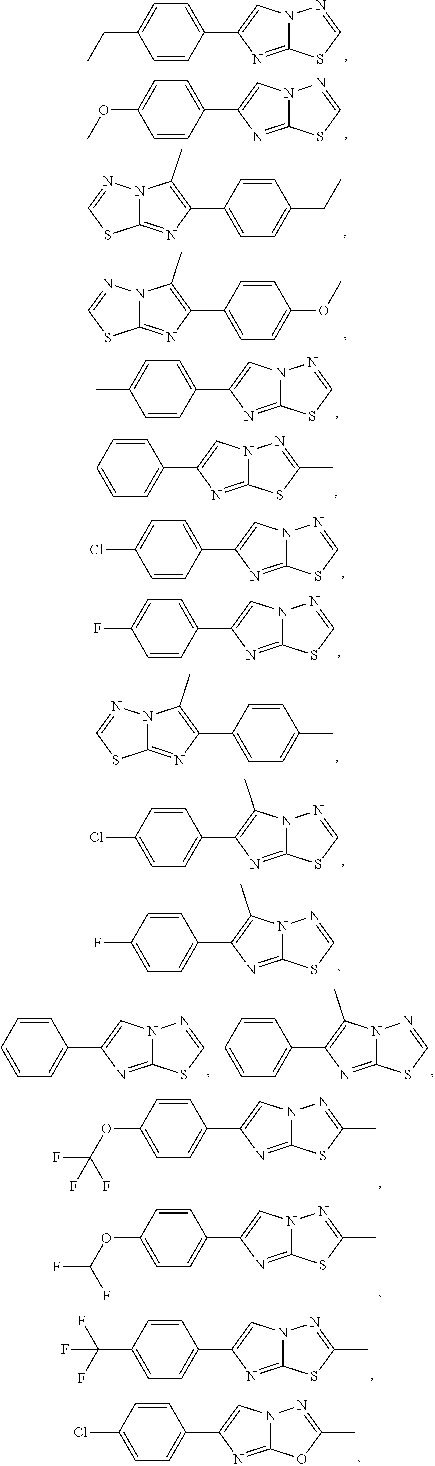

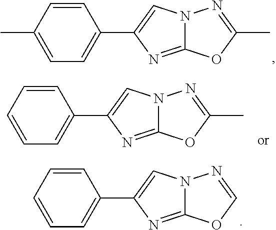

(5) A compound of one of the following formulae, or a salt and/or solvate thereof:

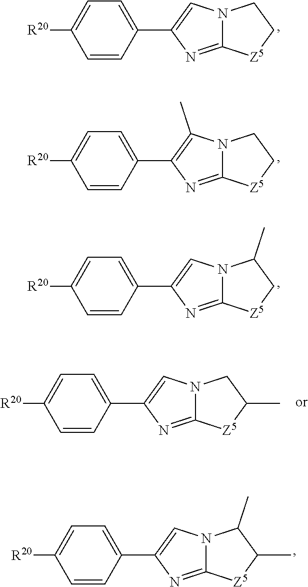

wherein:
R²⁰ is H, CH₃, CH₂CH₃, CH₂CH₂CH₃, F, Cl, CN, CF₃, OCF₃, OCHF₂, C(O)OH, C(O)H, C(O)OCH₃, C(O)OCH₂CH₃, C(O)OCH₂CH₂CH₃, OCH₃, OCH₂CH₃, OCH₂CH₂CH₃, C(O)CH₃, C(O)CH₂CH₃ or C(O)CH₂CH₂CH₃; and
Z⁵ is O or S, and
provided that the compound is not:

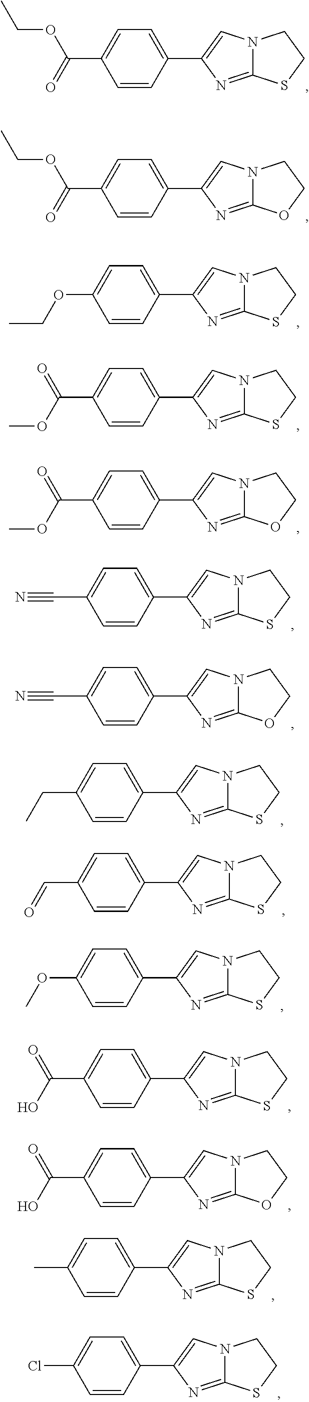

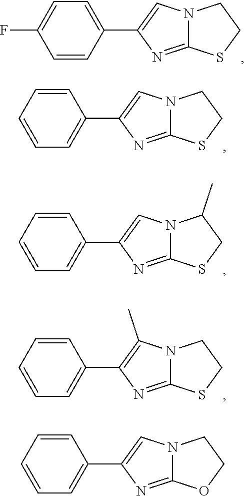

(6) A compound of one of the following formulae, or a salt and/or solvate thereof:

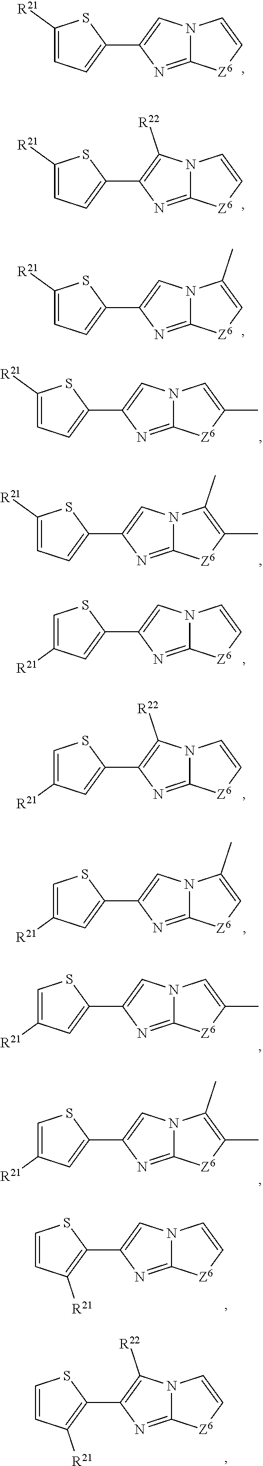

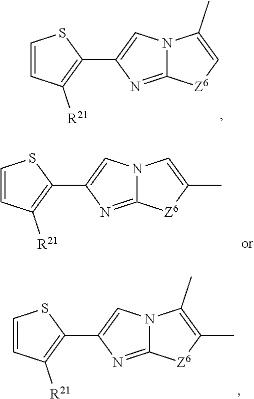

wherein:
R²¹ is H, CH₃, F or Cl;
R²² is CH₃ or C(O)H; and
Z⁶ is O or S, and
provided that the compound is not:

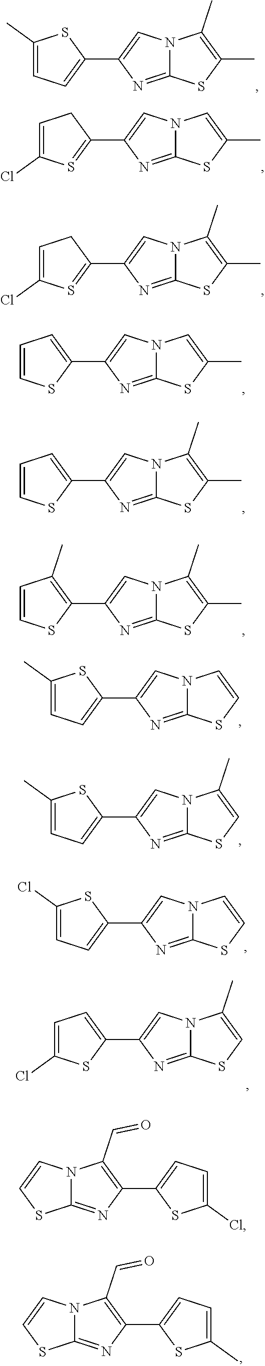

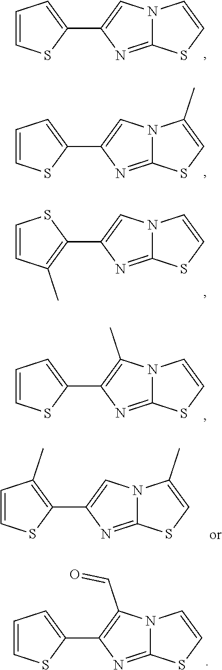

(7) A compound of one of the following formulae, or a salt and/or solvate thereof:

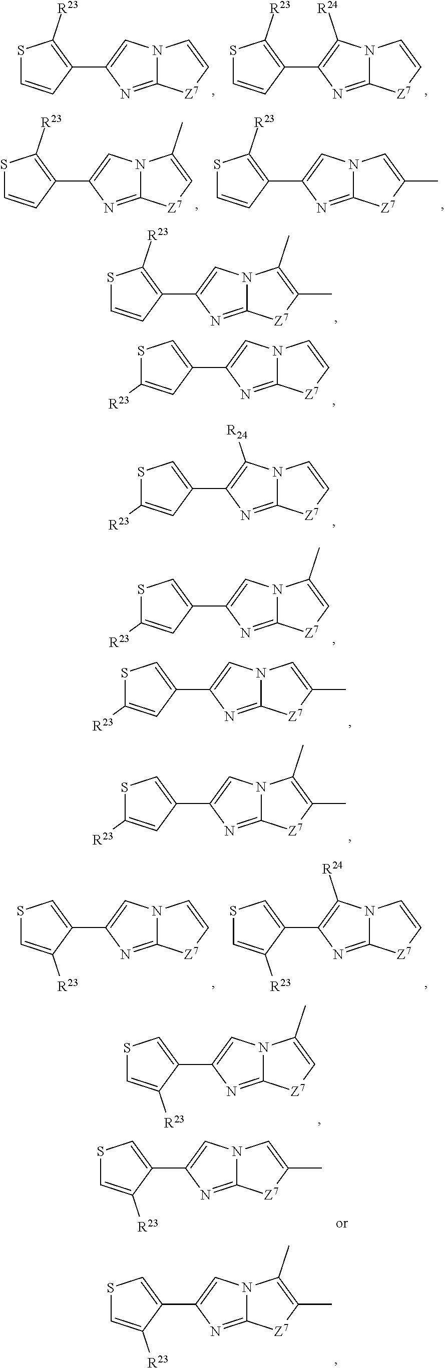

wherein:
R²³ is H, CH₃, F or Cl;
R²⁴ is CH₃ or C(O)H; and
Z⁷ is O or S, and
provided that the compound is not:

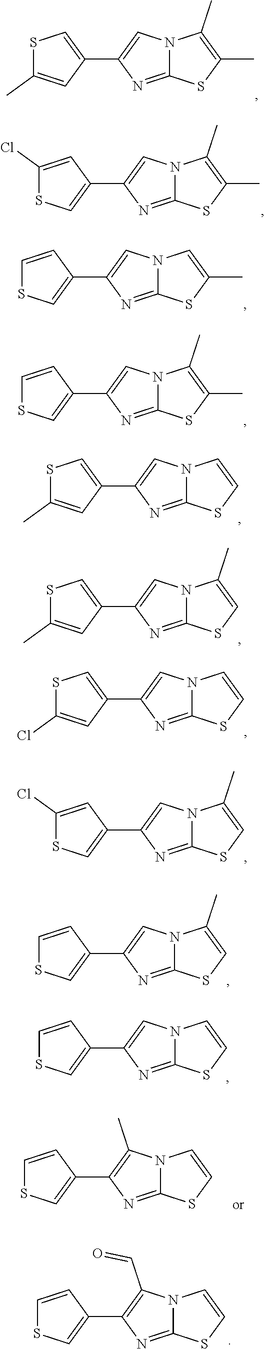

The following non-limiting examples are illustrative of the present application. As is apparent to those skilled in the art, many of the details of the examples may be changed while still practicing the methods, compositions and kits described herein.

### V. Examples

### Preparation of Compounds of Formula I

### Commercial Chemical Sources

In some embodiments, the compounds of the application useful in the present application are available from commercial sources such as Sigma, ChemBridge, Princeton, ChemDiv, Enamine, Life Chemicals, Maybridge, Otava, Specs, TimTec, Vitas-M, AKL Research, Key Organics, and MolPort. Compounds 1-6, 1-7, 1-10, 1-12, 1-18, 1-23, 1-26, 1-27, 1-30 to 1-32, 1-34, 1-37, 1-38, 1-41, 1-44, 1-45, 1-48, 1-49 and 1-87 to 1-159 were obtained from commercial sources.

### General Methods for Compound Synthesis and Characterization

Work-up and isolation of compounds was performed using standard benchtop techniques. All commercial reagents were purchased from chemical suppliers (Sigma-Aldrich, Combi-Blocks, Alfa Aesar, or Strem Chemicals) and used without further purification. Dry solvents were obtained using standard procedures (THF was distilled over sodium/benzophenone, dichloromethane was distilled over calcium hydride). Reactions were monitored using thin-layer chromatography (TLC) on EMD Silica Gel 60 F254 plates. Visualization was performed under UV light (254 nm) or using potassium permanganate (KMnO₄) or I₂ stain. Flash column chromatography was performed on Siliaflash P60 40-63 μm silica gel purchased from Silicycle.

NMR characterization data was obtained at 293K on a Varian Mercury 300 MHz, Varian Mercury 400 MHz, Bruker Advance Ill 400 MHz, Agilent DD2 500 MHz equipped with a 5 mm Xses cold probe or Agilent DD2 600 MHz. ¹H spectra were referenced to the residual solvent signal (CDCl₃=7.26 ppm, DMSO-d₆=2.50 ppm). ¹³C{¹H} spectra were referenced to the residual solvent signal (CDCl₃=77.16 ppm, DMSO-d₆=39.52 ppm). Data for ¹H NMR are reported as follows: chemical shift (δ ppm), multiplicity (s=singlet, d=doublet, t=triplet, q=quartet, m=multiplet), coupling constant (Hz), integration. NMR spectra were recorded at the University of Toronto Department of Chemistry NMR facility.

Infrared spectra were recorded on a Perkin-Elmer Spectrum 100 instrument equipped with a single-bounce diamond/ZnSe ATR accessory in the solid state and are reported in wavenumber (cm⁻¹) units.

Melting point ranges were done on a Fisher-Johns Melting Point Apparatus and are reported uncorrected.

High resolution mass spectra (HRMS) were recorded at the Advanced Instrumentation for Molecular Structure (AIMS) in the Department of Chemistry at the University of Toronto.

### Synthesis of 6-(4-chlorophenyl)imidazo[2,1-b]oxazole (compound 1-47)

Step 1: A solution of 2-amino-oxazole (0.150 g, 1.79 mol) in acetonitrile (2 ml) was slowly added to a solution of 2-bromo-1-(4-chlorophenyl)ethanone (0.84 g, 0.36 mol) in THE (3 ml). The reaction mixture was stirred at room temperature for 24 hrs then cooled to 0° C. A precipitate formed and was filtered off. The solid was washed with three portions of 30 ml of cold acetonitrile and dried at 50° C. under vacuum, yielding 0.402 g (71%) of 1-(4-chlorophenyl)-2-(2-iminooxazol-3-yl)-ethanone hydrobromide as an off-white solid.

Step 2: The 1-(4-chlorophenyl)-2-(2-iminooxazol-3-yl)-ethanone hydrobromide salt (0.200 g, 0.63 mol) was introduced in a microwave vial and flushed with nitrogen. Anhydrous toluene (3 ml) was added and the mixture was cooled to −10° C. and TiCl₄ (0.0346 ml, 0.315 mol) was added. The reaction becomes deep red and a dark precipitate was formed. The reaction mixture was allowed to warm to room temperature. The mixture was then brought to reflux and kept a reflux for 3.5 hrs. The reaction mixture was cooled to room temperature. The toluene was decanted and iced water (10 ml) was added to the residue (caution), which turned from dark brown to beige. The resulting suspension treated with saturated aqueous sodium carbonate solution until pH 8. A saturated aqueous sodium chloride solution was added (20 ml), followed by 40 ml of ethyl acetate and the mixture was stirred vigorously for one hour. The organic phase was separated and the aqueous phase was further extracted with two portions of 40 ml of ethyl acetate. The organic extracts were dried over sodium sulfate, concentrated onto celite and purified on silca gel (ISCO with ethylacetate/hexane 0-50%) to give the title compound as a white solid (0.055 g, 46% yield). 1 H NMR (500 MHz, chloroform-d) δ=7.64 (d, J=8.6 Hz, 2H), 7.33-7.30 (m, 1H), 7.27 (d, J=8.7 Hz, 2H), 7.25 (d, J=1.7 Hz, 1H), 7.21 (s,1 H).

### Synthesis and Characterization of Compounds 1-3 to 1-5, 1-8, 1-9, I-11, 1-13 to I-17, 1-19 to 1-22, 1-24, 1-25, 1-28, 1-29, 1-33, 1-35, 1-36, 1-39, 1-40, 1-42, 1-43

### Step 1. Synthesis of propiophenones

### Step 1.1. Synthesis of para-propiophenones

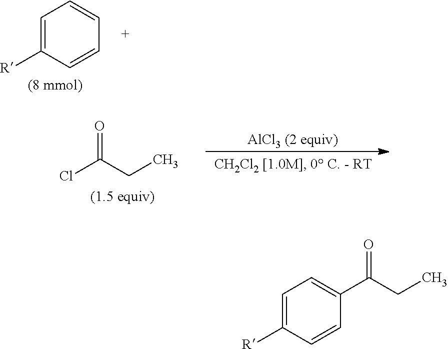

Non-commercially available para-substituted propiophenones (wherein R′ is as defined for the substituent groups on A in the compounds of Formula I) were prepared by electrophilic aromatic substitution using a literature procedure (Yates, C. M. WO 2018/165520 A1, 2018). The crude material was used in the next step without further purification.

### Step 1.2. Synthesis of ortho- and meta-parapropiophenones

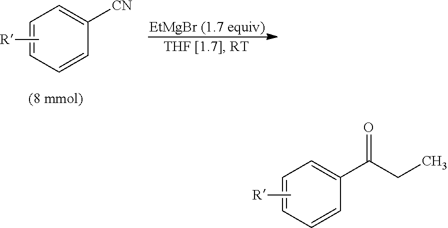

To a flame-dried round bottom flask under Ar was added the benzonitrile (wherein R′ is as defined for the substituent groups on A in the compounds of Formula I, 8 mmol, 1 equiv) then dry THE (4.6 mL). Ethylmagnesium bromide (4.6 mL, [3.0 M] in diethyl ether) was added slowly to the solution at room temperature. The reaction was stirred at room temperature until consumption of the nitrile was evident by TLC. Water (2 mL) was added slowly, followed by 4 M aqueous HCl (6 mL) and the mixture was stirred. After 1 hour, ethyl acetate and water were added to the reaction vessel and the mixture was transferred to a separatory funnel. After separation of the phases, the organic phase was washed with water then saturated sodium chloride, dried over MgSO₄, and concentrated on a rotary evaporator. The ortho- or meta-propiophenone was used in the next step without further purification.

### Step 1.3. Synthesis of 2-bromopropiophenone

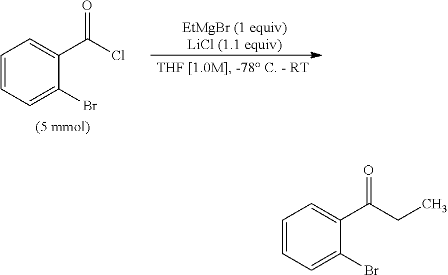

2-bromopropiophenone was prepared according to a literature procedure (Janody, S.; Jazzar, et al. Chem. -A Eur. J. 2014, 20 (35), 11084-11090).

### Step 2. Synthesis of α-bromoketones

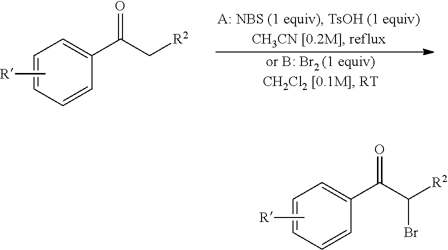

Method A (for R²=H): 2-bromo-acetophenone analogues were synthesized from the corresponding commercially available acetophenone according to literature procedures (Chundawat, T. S.; et al. Med. Chem. Res. 2016, 25 (10), 2335-2348).

Method B (for R²=CH₃): 2-bromo-propiophenone analogues were synthesized according to literature procedures (Lagoja, I. M. et al. J. Med. Chem. 2003, 46 (8), 1546-1553). The crude material was used in the next step without further purification.

### Step 3. Synthesis of imidazo[2,1-b]thiazoles

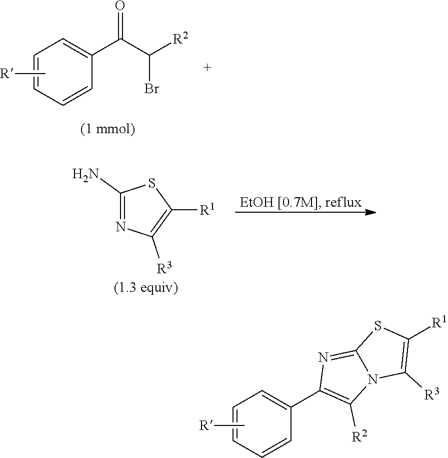

The imidazo[2,1-b]thiazoles were prepared according to a modified literature procedure (Pyl, T. et al. Justus Liebigs Ann. Chem. 1961, 643 (1), 145-153). To a 2 dram vial was added the α-bromoketone (1 mmol, 1 equiv), 2-aminothiazole (1.3 mmol, 1.3 equiv), and EtOH (3 mL) and the reaction mixture was stirred at reflux until disappearance of the α-bromoketone was evident by TLC. The mixture was concentrated, then purified by column chromatography using the given eluent to provide the imidazo[2,1-b]thiazole. Using the above procedures, the following compounds were prepared:

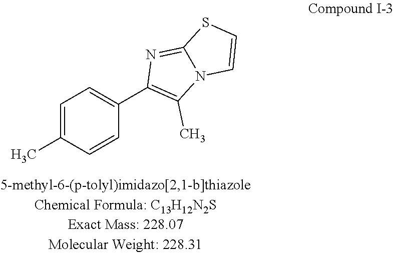 [SMILES: Cc1ccc(-c2nc3sccn3c2C)cc1]

Purified using pentanes-EtOAc (16:4 to 15:5 v:v). Yellow solid (56%, MP=96-99° C.). ¹H NMR (CDCl₃, 500 MHz): 7.61 (d, J=8.2 Hz, 2H), 7.29 (d, J=4.4 Hz, 1H), 7.24 (dt, J=7.9, 0.7 Hz, 2H), 6.81 (dd, J=4.5, 0.8 Hz, 1H), 2.59 (s, 3H), 2.39 (s, 3H). ¹³C{¹H}-NMR (CDCl₃, 125 MHz): 147.4, 143.2, 136.7, 132.2, 129.3, 127.3, 117.3, 116.9, 112.2, 21.4, 10.8. IR (neat): 3088, 2919, 2853, 2006, 1542, 1472, 1452, 1366, 1245, 1094, 819, 719, 681. Mass: ESI+, calc. for C₁₃H₁₃N₂S 229.07994 [M+H]⁺, found 229.07997.

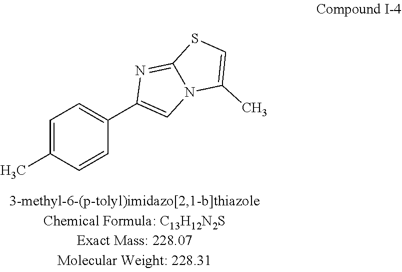

Purified using pentanes-EtOAc (16:4 to 15:5 v:v). Yellow solid (31%). ¹H NMR (CDCl₃, 500 MHz): 7.74 (d, J=8.2 Hz, 2H), 7.59 (d, J=1.1 Hz, 1H), 7.21 (d, J=7.8 Hz, 2H), 6.40 (tq, J=2.5, 1.5 Hz, 1H), 2.42 (q, J=1.7 Hz, 3H), 2.37 (s, 3H). ¹³C{¹H}-NMR (CDCl₃, 125 MHz): 149.7, 147.9, 137.1, 131.6, 129.4, 127.8, 125.2, 106.5, 105.7, 21.3, 13.5.

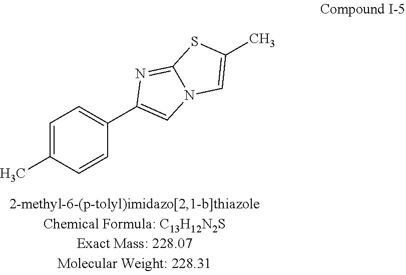

Purified using pentanes-EtOAc (16:4 to 15:5 v:v). Pale yellow solid (33%, MP=154-156° C.). ¹H NMR (CDCl₃, 500 MHz): 7.69 (d, J=8.2 Hz, 2H), 7.58 (s, 1H), 7.20 (d, J=7.9 Hz, 2H), 7.12 (q, J=1.4 Hz, 1H), 2.42 (d, J=1.5 Hz, 3H), 2.36 (s, 3H). ¹³C{¹H}-NMR (CDCl₃, 125 MHz): 149.6, 146.7, 137.1, 131.4, 129.5, 126.5, 125.1, 115.2, 107.4, 21.4, 14.2. IR (neat): 3105, 2923, 2854, 1548, 1469, 1192, 824, 787, 735. Mass: ESI+, calc. for C₁₃H₁₃N₂S 229.07994 [M+H]⁺, found 229.07939.

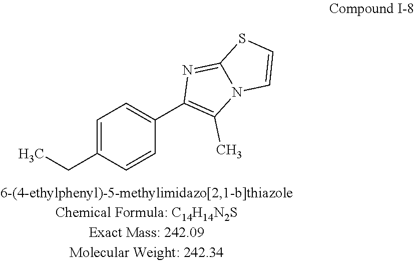 [SMILES: CCc1ccc(-c2nc3sccn3c2C)cc1]

Purified using pentanes-EtOAc (16:4 to 15:5 v:v). Yellow solid (30%, MP=61-65° C.). ¹H NMR (CDCl₃, 400 MHz): 7.63 (d, J=8.2 Hz, 2H), 7.29 (d, J=4.5 Hz, 1H), 7.27 (d, J=8.2 Hz, 2H), 6.81 (d, J=4.5 Hz, 1H), 2.69 (q, J=7.6 Hz, 2H), 2.59 (s, 3H), 1.27 (t, J=7.6 Hz, 3H). ¹³C{¹H}-NMR (CDCl₃, 125 MHz): 147.4, 143.3, 143.0, 132.5, 128.1, 127.4, 117.3, 116.9, 112.2, 28.7, 15.7, 10.9. IR (neat): 3067, 2961, 2916, 2871, 2011, 1548, 1480, 1010, 845, 718, 663. Mass: ESI+, calc. for C₁₄H₁₅N₂S 243.09559 [M+H]⁺, found 243.09594.

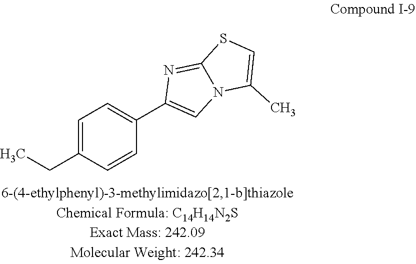 [SMILES: CCc1ccc(-c2cn3c(C)csc3n2)cc1]

Purified using pentanes-EtOAc (16:4 v:v). Orange solid (26%, MP=74-77° C.). ¹H NMR (CDCl₃, 500 MHz): 7.76 (d, J=8.2 Hz, 2H), 7.58 (s, 1H), 7.24 (d, J=8.6 Hz, 2H), 6.39 (q, J=1.3 Hz, 1H), 2.67 (q, J=7.6 Hz, 2H), 2.41 (d, J=1.3 Hz, 3H), 1.26 (t, J=7.6 Hz, 3H). ¹³C{¹H}-NMR (CDCl₃, 125 MHz): 149.8, 148.0, 143.6, 131.8, 128.3, 127.8, 125.3, 106.6, 105.7, 28.8, 15.7, 13.5. IR (neat): 3109, 2964, 2930, 2865, 2007, 1470, 1415, 1185, 835, 743, 694. Mass: ESI+, calc. for C₁₄H₁₅N₂S 243.09559 [M+H]⁺, found 243.09610.

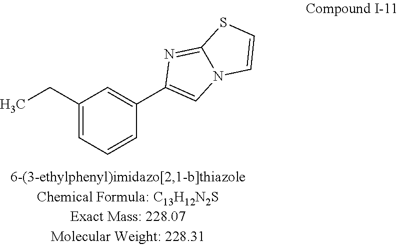 [SMILES: CCc1cccc(-c2cn3ccsc3n2)c1]

Purified using pentanes-EtOAc (15:5 v:v). Yellow oil (46%).1H NMR (CDCl₃, 500 MHz): 7.75-7.71 (m, 2H), 7.60 (d, J=7.7 Hz, 1H), 7.42 (q, J=4.4 Hz, 1H), 7.31 (t, J=7.6 Hz, 1H), 7.13 (ddt, J=7.7, 1.9, 1.0 Hz, 1H), 6.81 (q, J=4.6 Hz, 1H), 2.70 (q, J=7.6 Hz, 2H), 1.28 (t, J=7.6 Hz, 3H). ¹³C{¹H}-NMR (CDCl₃, 125 MHz): 150.2, 148.2, 144.9, 134.1, 128.8, 127.2, 125.0, 122.7, 118.6, 112.6, 108.0, 29.1, 15.8. IR (neat): 3115, 2967, 2933, 2007, 1613, 1555, 1462, 1212, 856, 801, 722. Mass: ESI+, calc. for C₁₃H₁₃N₂S 229.07994 [M+H]⁺, found 229.08017.

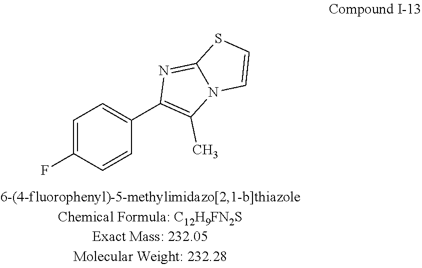 [SMILES: Cc1c(-c2ccc(F)cc2)nc2sccn12]

Purified using pentanes-EtOAc (15:5 to 10:10 v:v). Yellow solid (27%, MP=70-73° C.). ¹H NMR (CDCl₃, 500 MHz): 7.66 (dd, J=8.9, 5.4 Hz, 2H), 7.29 (d, J=4.5 Hz, 1H), 7.11 (t, J=8.8 Hz, 2H), 6.82 (d, J=4.5 Hz, 1H), 2.56 (s, 3H). ¹³C{¹H}-NMR (CDCl₃, 125 MHz): 162.0 (d, J=245.9 Hz), 147.6, 142.4, 131.2 (d, J=3.2 Hz), 129.0 (d, J=8.0 Hz), 117.4, 116.8, 115.5 (d, J=21.4 Hz), 112.4, 10.7. ¹⁹F{¹H}-NMR (CDCl₃, 375 MHz): -114.7. IR (neat): 3046, 2916, 2007, 1503, 1222, 1092, 842, 715, 660. Mass: ESI+, calc. for C₁₂H₁₀FN₂S 233.05487 [M+H]⁺, found 233.05438.

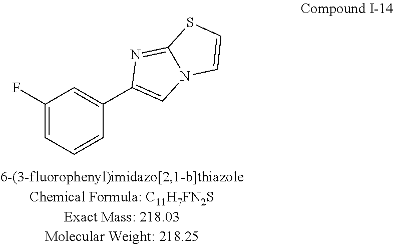 [SMILES: Fc1cccc(-c2cn3ccsc3n2)c1]

Purified using pentanes-EtOAc (15:5 v:v). Yellow solid (41%). ¹H-NMR (CDCl₃, 500 MHz): 7.74 (s, 1H), 7.59 (ddd, J=7.8, 1.6, 1.0 Hz, 1H), 7.53 (ddd, J=10.3, 2.6, 1.5 Hz, 1H), 7.43 (d, J=4.5 Hz, 1H), 7.35 (td, J=8.0, 6.0 Hz, 1H), 6.97 (tdd, J=8.3, 2.6, 1.0 Hz, 1H), 6.84 (d, J=4.5 Hz, 1H). ¹³C{¹H}-NMR (CDCl₃, 125 MHz): 163.4 (d, J=244.9 Hz), 150.5, 146.8 (d, J=2.9 Hz), 136.4 (d, J=8.3 Hz), 130.3 (d, J=8.5 Hz), 120.9 (d, J=2.8 Hz), 118.6, 114.3 (d, J=21.3 Hz), 113.1, 112.2 (d, J=22.8 Hz), 108.6. ¹⁹F{¹H}-NMR (CDCl₃, 375 MHz): -113.3.

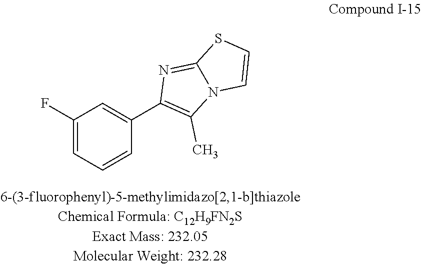 [SMILES: Cc1c(-c2cccc(F)c2)nc2sccn12]

Purified using pentanes-EtOAc (14:6 v:v). Pale orange solid (37%, MP=125-128° C.). ¹H-NMR (CDCl₃, 500 MHz): 7.50 (ddd, J=7.8, 1.6, 1.0 Hz, 1H), 7.44 (ddd, J=10.4, 2.6, 1.6 Hz, 1H), 7.38 (td, J=8.0, 6.0 Hz, 1H), 7.31 (d, J=4.5 Hz, 1H), 6.98 (tdd, J=8.4, 2.6, 1.0 Hz, 1H), 6.85 (d, J=4.5 Hz, 1H), 2.61 (s, 3H). ¹³C{¹H}-NMR (CDCl₃, 125 MHz): 163.2 (d, J=244.7 Hz), 147.7, 142.0, 137.3 (d, J=8.3 Hz), 130.1 (d, J=8.5 Hz), 122.9 (d, J=2.8 Hz), 118.2, 116.8, 114.1 (d, J=22.7 Hz), 113.8 (d, J=21.2 Hz), 112.8, 10.9. ¹⁹F{¹H}-NMR (CDCl₃, 375 MHz): -113.3. IR (neat): 3063, 2011, 1589, 1438, 1270, 1208, 873, 787, 708, 660. Mass: DART+, calc. for C₁₂H₁₀N₂FS 233.05432 [M+H]⁺, found 233.05410.

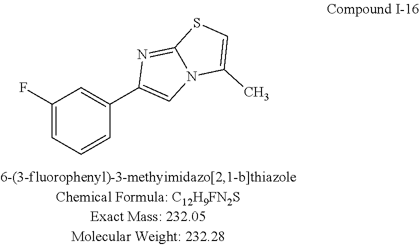 [SMILES: Cc1csc2nc(-c3cccc(F)c3)cn12]

Purified using pentanes-EtOAc (15:5 v:v). Yellow solid (38%, MP=132-135° C.). ¹H-NMR (CDCl₃, 500 MHz): 7.63 (s, 1H), 7.61 (ddd, J=7.8, 1.5, 0.9 Hz, 1H), 7.55 (ddd, J=10.2, 2.5, 1.5 Hz, 1H), 7.35 (td, J=8.0, 6.0 Hz, 1H), 6.96 (tdd, J=8.4, 2.6, 1.0 Hz, 1H), 6.44 (q, J=1.3 Hz, 1H), 2.43 (d, J=1.3 Hz, 3H). ¹³C{¹H}-NMR (CDCl₃, 125 MHz): 163.3 (d, J=244.8 Hz), 149.9, 146.5, 136.4 (d, J=8.4 Hz), 130.1 (d, J=8.4 Hz), 127.7, 120.7 (d, J=2.8 Hz), 114.1 (d, J=21.3 Hz), 112.1 (d, J=22.9 Hz), 107.1, 106.5, 13.4. ¹⁹F{¹H}-NMR (CDCl₃, 375 MHz): -113.4. IR (neat): 3087, 2919, 2004, 1617, 1472, 1294, 1175, 869, 715, 660. Mass: DART+, calc. for C₁₂H₁₀N₂FS 233.05432 [M+H]⁺, found 233.05483.

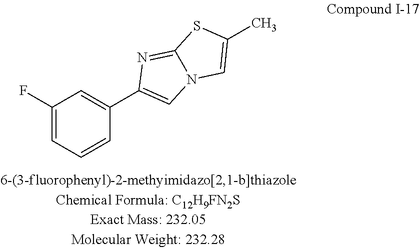 [SMILES: Cc1cn2cc(-c3cccc(F)c3)nc2s1]

Purified using pentanes-EtOAc (15:5 v:v). Yellow solid (35%, MP=93-98° C.). ¹H-NMR (CDCl₃, 500 MHz): 7.60 (s, 1H), 7.56 (ddd, J=7.8, 1.6, 0.9 Hz, 1H), 7.50 (ddd, J=10.3, 2.6, 1.5 Hz, 1H), 7.33 (td, J=7.9, 6.0 Hz, 1H), 7.12 (q, J=1.4 Hz, 1H), 6.94 (tdd, J=8.4, 2.6, 1.0 Hz, 1H), 2.41 (d, J=1.5 Hz, 3H). ¹³C{¹H}-NMR (CDCl₃, 125 MHz): 163.4 (d, J=244.5 Hz), 149.9, 145.5 (d, J=2.8 Hz), 136.6 (d, J=8.4 Hz), 130.2 (d, J=8.4 Hz), 127.0, 120.7 (d, J=2.8 Hz), 115.2, 114.0 (d, J=21.3 Hz), 112.0 (d, J=23.0 Hz), 108.3, 14.2. ¹⁹F{¹H}-NMR (CDCl₃, 375 MHz): -113.4. IR (neat): 3153, 3077, 2919, 2007, 1616, 1589, 1442, 1174, 872, 783, 732, 667. Mass: DART+, calc. for C₁₂H₁₀₁N₂FS 233.05432 [M+H]⁺, found 233.05444.

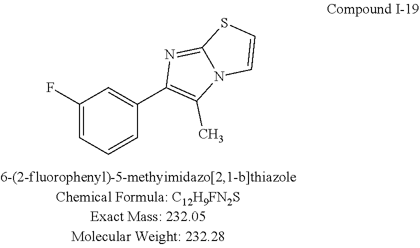 [SMILES: Cc1c(-c2cccc(F)c2)nc2sccn12]

Purified using pentanes-EtOAc (14:6 to 10:10 v:v). Pale yellow solid (26%, MP=84-88° C.). ¹H-NMR (CDCl₃, 500 MHz): 7.68 (td, J=7.6, 1.8 Hz, 1H), 7.34-7.29 (m, 2H), 7.22 (td, J=7.5, 1.2 Hz, 1H), 7.14 (ddd, J=10.6, 8.2, 1.2 Hz, 1H), 6.85 (d, J=4.5 Hz, 1H), 2.45 (d, J=2.7 Hz, 3H). ¹³C{¹H}-NMR (CDCl₃, 125 MHz): 159.5 (d, J=247.1 Hz), 147.9, 137.9, 131.6 (d, J=3.5 Hz), 129.2 (d, J=8.2 Hz), 124.4 (d, J=3.5 Hz), 122.8 (d, J=14.6 Hz), 119.9, 116.9, 115.9 (d, J=22.6 Hz), 112.6, 10.4. ¹⁹F{¹H}-NMR (CDCl₃, 375 MHz): -114.8. IR (neat): 3132, 3043, 2007, 1493, 1442, 1222, 1092, 760, 719, 663. Mass: DART+, calc. for C₁₂H₁₀N₂FS 233.05432 [M+H]⁺, found 233.05369.

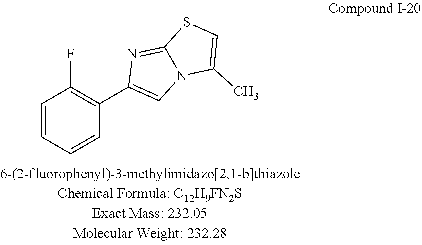 [SMILES: Cc1csc2nc(-c3ccccc3F)cn12]

Purified using pentanes-EtOAc (15:5 v:v). Yellow solid (31%, MP=75-79° C.). ¹H-NMR (CDCl₃, 500 MHz): 8.24-8.20 (m, 1H), 7.81 (d, J=3.8 Hz, 1H), 7.25-7.20 (m, 2H), 7.14-7.09 (m, 1H), 6.42 (q, J=1.3 Hz, 1H), 2.44 (d, J=1.3 Hz, 3H). ¹³C{¹H}-NMR (CDCl₃, 125 MHz): 159.8 (d, J=248.2 Hz), 149.5, 141.2 (d, J=2.4 Hz), 128.3 (d, J=4.1 Hz), 128.2 (d, J=8.5 Hz), 127.9, 124.6 (d, J=3.2 Hz), 122.1 (d, J=12.6 Hz), 115.7 (d, J=22.1 Hz), 110.4 (d, J=15.7 Hz), 107.0, 13.5. ¹⁹F{¹H}-NMR (CDCl₃, 375 MHz): -114.3. IR (neat): 3071, 2923, 2007, 1480, 1435, 1185, 1068, 763, 694. Mass: DART+, calc. for C₁₂H₁₁N₂FS 233.05432 [M+H]⁺, found 233.05448.

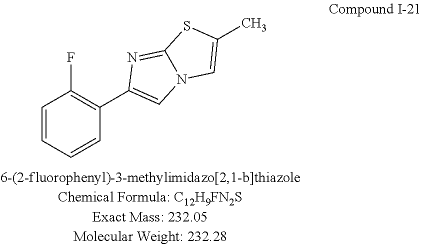 [SMILES: Cc1cn2cc(-c3ccccc3F)nc2s1]

Purified using pentanes-EtOAc (16:4 v:v). Pale yellow solid (32%, MP=104-108° C.). ¹H-NMR (CDCl₃, 500 MHz): 8.22-8.15 (m, 1H), 7.80 (d, J=3.9 Hz, 1H), 7.24-7.19 (m, 2H), 7.14 (q, J=1.4 Hz, 1H), 7.12-7.07 (m, 1H), 2.42 (d, J=1.4 Hz, 3H). ¹³C{¹H}-NMR (CDCl₃, 125 MHz): 159.7 (d, J=248.1 Hz), 149.3, 139.9 (d, J=2.6 Hz), 128.1 (d, J=8.5 Hz), 128.1 (d, J=4.0 Hz), 126.9, 124.5 (d, J=3.2 Hz), 122.1 (d, J=12.6 Hz), 115.7 (d, J=22.1 Hz), 115.2, 112.0 (d, J=15.7 Hz), 14.2. ¹⁹F{¹H}-NMR (CDCl₃, 375 MHz): -114.2. IR (neat): 3053, 2927, 2011, 1480, 1435, 1212, 1065, 759, 739, 715. Mass: DART+, calc. for C₁₂H₁₀N₂FS 233.05432 [M+H]⁺, found 233.05447.

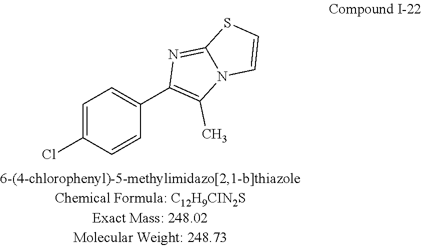 [SMILES: Cc1c(-c2ccc(Cl)cc2)nc2sccn12]

Purified using pentanes-EtOAc (15:5 to 10:10 v:v). Pale orange solid (25%, MP=135-137° C.). ¹H NMR (CDCl₃, 500 MHz): 7.65 (d, J=8.6 Hz, 2H), 7.39 (d, J=8.7 Hz, 2H), 7.30 (d, J=4.5 Hz, 1H), 6.85 (d, J=4.5 Hz, 1H), 2.59 (s, 3H). ¹³C{¹H}-NMR (CDCl₃, 125 MHz): 147.7, 142.1, 133.6, 132.8, 128.8, 128.6, 117.9, 116.8, 112.7, 10.9. IR (neat): 3105, 2926, 2007, 1541, 1490, 1400, 1092, 1007, 831. Mass: ESI+, calc. for C₁₂H₁₀ClN₂S 249.02532 [M+H]⁺, found 249.02481.

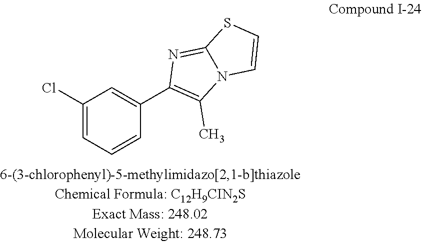 [SMILES: Cc1c(-c2cccc(Cl)c2)nc2sccn12]

Purified using pentanes-EtOAc (16:4 to 15:5 v:v). Pale orange solid (80%, MP=84-88° C.).1H NMR (CDCl₃, 500 MHz): 7.72 (t, J=1.9 Hz, 1H), 7.60 (dt, J=7.8, 1.4 Hz, 1H), 7.35 (t, J=7.8 Hz, 1H), 7.31 (dd, J=4.5, 0.6 Hz, 1H), 7.26 (ddd, J=8.0, 2.2, 1.1 Hz, 1H), 6.87-6.84 (m, 1H), 2.61 (d, J=0.6 Hz, 3H). ¹³C{¹H}-NMR (CDCl₃, 125 MHz): 147.79, 141.82, 136.92, 134.59, 129.87, 127.31, 126.94, 125.34, 118.24, 116.83, 112.81, 10.91. IR (neat): 3050, 2006, 1600, 1483, 1007, 811, 777, 698. Mass: ESI+, calc. for C₁₂H₁₀CIl₂S 249.02532 [M+H]⁺, found 249.02563.

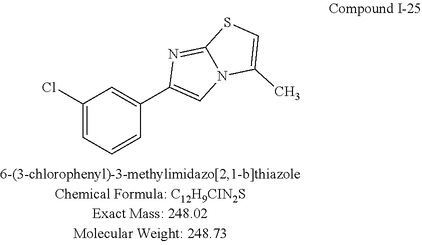 [SMILES: Cc1csc2nc(-c3cccc(Cl)c3)cn12]

Purified using pentanes-EtOAc (15:5 v:v). Yellow solid (42%). ¹H-NMR (CDCl₃, 500 MHz): 7.84 (ddd, J=2.1, 1.6, 0.4 Hz, 1H), 7.71 (ddd, J=7.7, 1.6, 1.1 Hz, 1H), 7.62 (s, 1H), 7.32 (td, J=7.9, 0.4 Hz, 1H), 7.23 (ddd, J=8.0, 2.1, 1.1 Hz, 1H), 6.43 (q, J=1.3 Hz, 1H), 2.42 (d, J=1.3 Hz, 3H). ¹³C{¹H}-NMR (CDCl₃, 125 MHz): 150.0, 146.2, 136.0, 134.7, 129.9, 127.7, 127.2, 125.2, 123.2, 107.2, 106.5, 13.4.

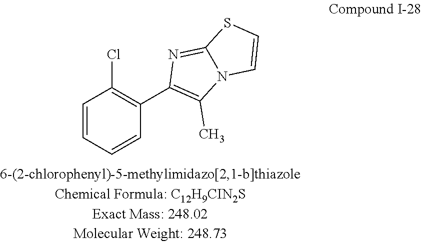 [SMILES: Cc1c(-c2ccccc2Cl)nc2sccn12]

Purified using pentanes-EtOAc (16:4 to 15:5 v:v). Orange solid (19%, MP=145-148° C.). ¹H NMR (CDCl₃, 400 MHz): 7.54-7.49 (m, 1H), 7.49-7.45 (m, 1H), 7.36-7.27 (m, 3H), 6.89-6.84 (m, 1H), 2.39 (s, 3H). ¹³C{¹H}-NMR (CDCl₃, 125 MHz): 147.5, 141.0, 133.7, 133.6, 132.6, 129.9, 129.2, 126.8, 119.7, 116.9, 112.7, 10.7. IR (neat): 3132, 3036, 2926, 2855, 2008, 1479, 1360, 1037, 859, 759, 722, 667. Mass: ESI+, calc. for C₁₂H₁₀CIl₂S 249.02532 [M+H]⁺, found 249.02559.

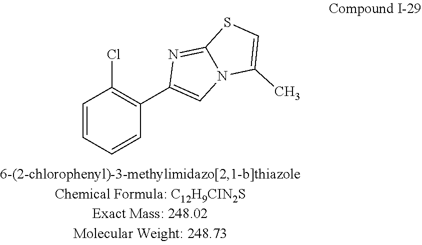 [SMILES: Cc1csc2nc(-c3ccccc3Cl)cn12]

Purified using pentanes-EtOAc (16:4 v:v). Yellow solid (28%, MP=82-85° C.). ¹H NMR (CDCl₃, 500 MHz): 8.21 (dd, J=7.9, 1.7 Hz, 1H), 8.05 (s, 1H), 7.44 (dd, J=8.0, 1.2 Hz, 1H), 7.35 (ddd, J=7.9, 7.3, 1.3 Hz, 1H), 7.21 (ddd, J=8.0, 7.3, 1.7 Hz, 1H), 6.44 (q, J=1.3 Hz, 1H), 2.45 (d, J=1.3 Hz, 3H). ¹³C{¹H}-NMR (CDCl₃, 125 MHz): 149.0, 143.7, 132.6, 130.9, 130.5, 130.4, 128.1, 128.0, 127.1, 110.7, 107.2, 13.6. IR (neat): 3060, 2919, 2007, 1473, 1404, 1195, 1041, 736, 688. Mass: ESI+, calc. for C₁₂H₁₀CIl₂S 249.02532 [M+H]⁺, found 249.02531.

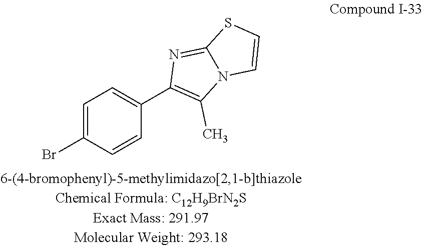 [SMILES: Cc1c(-c2ccc(Br)cc2)nc2sccn12]

Purified using pentanes-EtOAc (15:5 to 10:10 v:v). Pale yellow solid (27%, MP=146-149° C.). ¹H NMR (CDCl₃, 500 MHz): 7.61-7.57 (m, 2H), 7.56-7.52 (m, 2H), 7.32-7.28 (m, 1H), 6.85 (ddd, J=4.3, 2.9, 1.5 Hz, 1H), 2.58 (dd, J=1.7, 0.8 Hz, 3H). ¹³C{¹H}-NMR (CDCl₃, 125 MHz): 147.7, 142.1, 134.0, 131.7, 128.9, 120.9, 117.9, 116.8, 112.7, 10.9. IR (neat): 3101, 2926, 2011, 1538, 1486, 1401, 1006, 828, 712. Mass: ESI+, calc. for C₁₂H₁₀BrN₂S 292.97481 [M+H]⁺, found 292.97526.

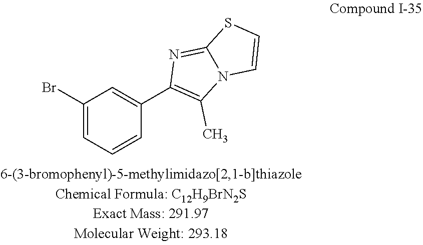 [SMILES: Cc1c(-c2cccc(Br)c2)nc2sccn12]

Purified using pentanes-EtOAc (15:5 to 14:6 v:v). Yellow solid (12%, MP=91-93° C.). ¹H NMR (CDCl₃, 500 MHz): 7.89 (t, J=1.8 Hz, 1H), 7.67-7.61 (m, 1H), 7.41 (dddd, J=8.5, 4.4, 2.1, 1.1 Hz, 1H), 7.33-7.27 (m, 2H), 6.88-6.82 (m, 1H), 2.62-2.57 (m, 3H). ¹³C{¹H}-NMR (CDCl₃, 125 MHz): 147.8, 141.7, 137.2, 130.2, 130.1, 129.8, 125.8, 122.8, 118.3, 116.8, 112.8, 10.9. IR (neat): 3047, 2927, 2011, 1596, 1483, 1367, 773, 698. Mass: DART+, calc. for C₁₂H₁₀BrN₂S 292.97426 [M+H]⁺, found 292.97413.

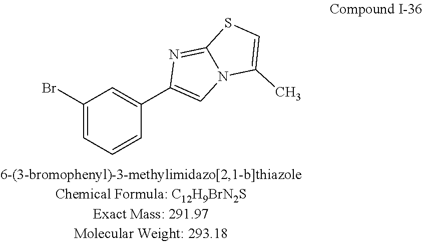 [SMILES: Cc1csc2nc(-c3cccc(Br)c3)cn12]

Purified using pentanes-EtOAc (15:5 v:v). Orange solid (29%, MP=95-98° C.). ¹H-NMR (CDCl₃, 500 MHz): 8.00 (t, J=1.8 Hz, 1H), 7.76 (ddd, J=7.8, 1.6, 1.0 Hz, 1H), 7.62 (s, 1H), 7.39 (ddd, J=7.9, 2.0, 1.0 Hz, 1H), 7.25 (t, J=7.9 Hz, 1H), 6.43 (q, J=1.3 Hz, 1H), 2.42 (d, J=1.3 Hz, 3H). ¹³C{¹H}-NMR (CDCl₃, 125 MHz): 150.2, 146.4, 136.5, 130.3, 130.3, 128.3, 127.8, 123.8, 123.0, 107.2, 106.6, 13.5. IR (neat): 3153, 3112, 2007, 1600, 1472, 1404, 1065, 893, 773, 729. Mass: DART+, calc. for C₁₂H₁₀BrN₂S 292.97426 [M+H]⁺, found 292.97453.

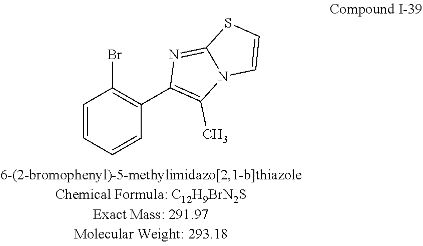 [SMILES: Cc1c(-c2ccccc2Br)nc2sccn12]

Purified using pentanes-EtOAc (15:5 v:v). Orange solid (22%, MP=144-147° C.). ¹H-NMR (CDCl₃, 500 MHz): 7.68-7.64 (m, 1H), 7.49-7.45 (m, 1H), 7.36 (td, J=7.5, 1.2 Hz, 1H), 7.31 (d, J=4.5 Hz, 1H), 7.22 (ddd, J=8.1, 7.4, 1.7 Hz, 1H), 6.85 (d, J=4.5 Hz, 1H), 2.38 (s, 3H). ¹³C{¹H}-NMR (CDCl₃, 125 MHz): 147.3, 142.8, 135.8, 133.0, 132.7, 129.4, 127.3, 123.9, 119.3, 116.9, 112.5, 10.7. IR (neat): 3132, 3036, 2927, 2007, 1476, 1425, 1356, 1007, 856, 766, 722, 664. Mass: DART+, calc. for C₁₂H₁₀N₂SBr 292.97426 [M+H]⁺, found 292.97354.

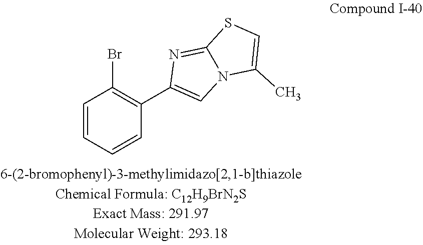 [SMILES: Cc1csc2nc(-c3ccccc3Br)cn12]

Purified using pentanes-EtOAc (16:4 to 15:5 v:v). Yellow solid (29%, MP=72-77° C.). ¹H-NMR (CDCl₃, 500 MHz): 8.08 (dd, J=7.9, 1.8 Hz, 1H), 8.06 (s, 1H), 7.64 (dd, J=8.0, 1.2 Hz, 1H), 7.41-7.36 (m, 1H), 7.14 (ddd, J=8.0, 7.3, 1.8 Hz, 1H), 6.44 (q, J=1.2 Hz, 1H), 2.45 (d, J=1.3 Hz, 3H). ¹³C{¹H}-NMR (CDCl₃, 125 MHz): 149.0, 144.9, 134.6, 133.8, 131.2, 128.5, 128.0, 127.7, 120.9, 110.3, 107.2, 13.6. IR (neat): 3064, 2008, 1473, 1293, 1181, 1020, 732, 670. Mass: DART+, calc. for C₁₂H₁₀N₂SBr 292.97426 [M+H]⁺, found 292.97354.

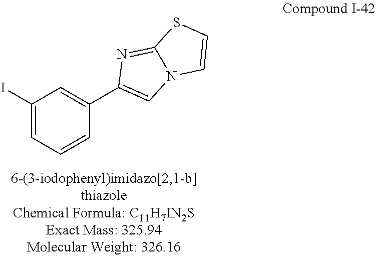 [SMILES: Ic1cccc(-c2cn3ccsc3n2)c1]

Purified using pentanes-EtOAc (16:4 to 15:5 v:v). Yellow solid (34%, MP=117-120° C.). ¹H-NMR (CDCl₃, 500 MHz): 8.20 (t, J=1.7 Hz, 1H), 7.77 (ddd, J=7.8, 1.7, 1.0 Hz, 1H), 7.73 (s, 1H), 7.60 (ddd, J=7.9, 1.8, 1.1 Hz, 1H), 7.43 (d, J=4.5 Hz, 1H), 7.12 (td, J=7.8, 0.4 Hz, 1H), 6.84 (d, J=4.5 Hz, 1H). ¹³C{¹H}-NMR (CDCl₃, 125 MHz): 150.5, 146.4, 136.4, 136.4, 134.2, 130.5, 124.4, 118.6, 113.0, 108.5, 94.9. IR (neat): 3143, 3116, 3060, 2007, 1548, 1462, 1195, 1058, 852, 787, 722. Mass: DART+, calc. for C₁₁H₇IN₂S 326.94474 [M+H]⁺, found 326.94479.

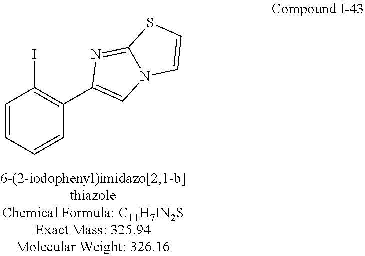 [SMILES: Ic1ccccc1-c1cn2ccsc2n1]

Purified using DCM-EtOAc (97:3 to 96:4 v:v). Pale orange solid (27%, MP=62-64° C.). ¹H-NMR (CDCl₃, 500 MHz): 8.05 (s, 1H), 7.98-7.94 (m, 1H), 7.81 (dd, J=7.8, 1.6 Hz, 1H), 7.46 (d, J=4.5 Hz, 1H), 7.40 (ddd, J=7.8, 7.3, 1.3 Hz, 1H), 6.99 (ddd, J=7.9, 7.3, 1.7 Hz, 1H), 6.85 (d, J=4.5 Hz, 1H). ¹³C{¹H}-NMR (CDCl₃, 125 MHz): 149.3, 148.0, 140.5, 138.8, 131.1, 129.0, 128.3, 118.7, 112.9, 111.4, 96.4. IR (neat): 3160, 3112, 3009, 2011, 1473, 1191, 1006, 766, 732. Mass: DART+, calc. for C₁₁H₈N₂SI 326.94474 [M+H]⁺, found 326.94408.

### Synthesis and Characterization of Compounds 1-46, 1-50 to 1-86 and 1-160 to I-163

### Step 1. Synthesis of (hetero)aryl ketones

### Step 1.1. Synthesis of 1-(4-(difluoromethoxy)phenyl)ethan-1-one

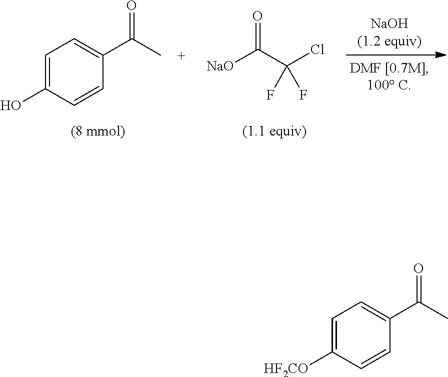

1-(4-(difluoromethoxy)phenyl)ethan-1-one was synthesized from the corresponding commercially available 4-hydroxyacetophenone according to literature procedure (Yates, C. M. WO 2018/165520 A1, 2018).

### Step 1.2. Synthesis of 1-(1-methoxynaphthalen-2-yl)ethan-1-one

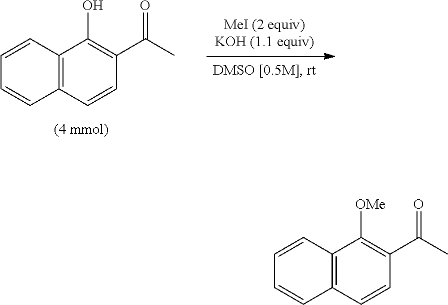

1-(1-methoxynaphthalen-2-yl)ethan-1-one was synthesized from the corresponding commercially available 1-(1-hydroxynaphthalen-2-yl)ethan-1-one according to literature procedure (Bolchi, C. et al. Bioorganic Med. Chem. 2004, 12 (18), 4937-4951).

### Step 1.3. Synthesis of 2-acetylpyridines

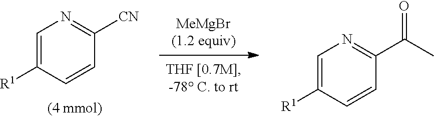

2-acetylpyridines were synthesized from the corresponding commercially available 2-cyanopyridines (wherein R′ is as defined for the substituent groups on A in the compounds of Formula I) according to literature procedure (Ikeda, E.; et al. EP1671941A1, 2006).

### Step 2. Synthesis of α-bromoketones

### Step 2.1. Synthesis of α-bromophenones

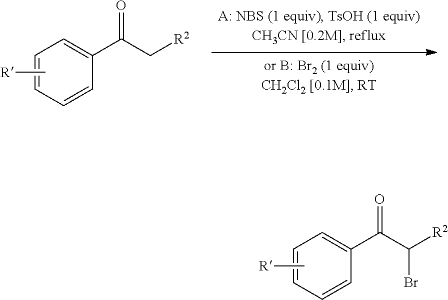

Method A (for R²=H): α-bromo-acetophenone analogues were synthesized from the corresponding commercially available acetophenone (wherein R′ is as defined for the substituent groups on A in the compounds of Formula I) according to literature procedures (Chundawat, T. S. et al. Med. Chem. Res. 2016, 25 (10), 2335-2348). The crude material was used in the next step without further purification.

Method B (for R²=CH₃): α-bromo-propiophenone analogues were synthesized from the corresponding commercially available propiophenone (wherein R′ is as defined for the substituent groups on A in the compounds of Formula I) according to literature procedures (Lagoja, I. M. et al. J. Med. Chem. 2003, 46 (8), 1546-1553) The crude material was used in the next step without further purification.

For R′=4-CN, R²=H and R′=4-CF₃, R²=H the commercially available α-bromoacetophenone was used.

### Step 2.2. Synthesis of α-bromopyridinones

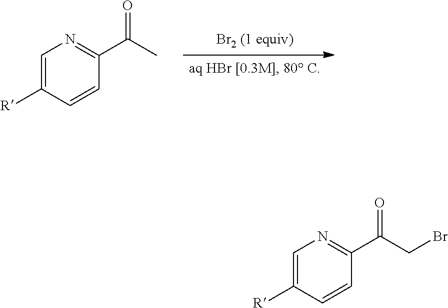

α-bromo-acetylpyridine analogues (wherein R′ is as defined for the substituent groups on A in the compounds of Formula I) were synthesized according to a modified literature procedure (Schuster, I.; Egger, H. U.S. Ser. No. 00/562,2982A, 1997). The substituted acetylpyridine (1 mmol, 1 equiv) was weighed into a 2 dram vial. Aqueous hydrobromic acid (48% solution, 2.4 mL) was added and the mixture was submerged in an oil bath at 80° C. To a separate vial was added bromine (0.05 mL, 1 equiv) followed by aqueous hydrobromic acid (48% solution, 0.65 mL), and this mixture was added dropwise to the reaction stirring at 80° C. After 1 hour, the mixture was cooled to room temperature and saturated NaHCO₃ was added until the pH was neutral. Ethyl acetate and water were added and the layers were separated. The organic phase was washed with an aqueous saturated solution of sodium thiosulfate then a saturated solution of brine and dried over magnesium sulfate. The solution was concentrated to afford the α-bromo-acetylpyridine, which was used in the next step without further purification.

### Step 2.3. Synthesis of α-bromothiophenones and 2-bromo-1-(1-methoxynaphthalen-2-yl)ethan-1-one

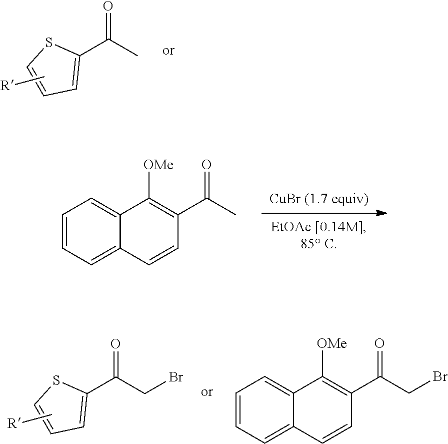

α-bromo-acetylthiophene analogues (wherein R′ is as defined for the substituent groups on A in the compounds of Formula I) were synthesized from the corresponding commercially available acetylthiophene according to a modified literature procedure (Schiffrer, E. S. et al. Medchemcomm 2019, 10 (11), 1958-1965). The synthesis of 2-bromo-1-(1-methoxynaphthalen-2-yl)ethan-1-one was accomplished using the same procedure. To a round bottom flask under argon was added the ketone (4 mmol, 1 equiv) followed by ethyl acetate (29 mL, 0.14 M). Copper (II) bromide (1.52 g, 6.8 mmol, 1.7 equiv) was added and the mixture was stirred at 85° C. overnight. The mixture was cooled to room temperature, filtered over a pad of celite then washed twice with a solution of saturated ammonium chloride. The organic layer was dried over MgSO₄ then concentrated to give the α-bromoketone, which was used in the next step without further purification.

### Step 3. Synthesis of imidazo[2,1-b]thiazoles

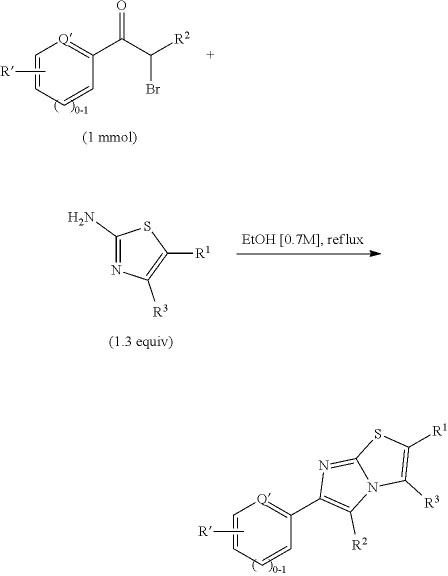

The imidazo[2,1-b]thiazoles were prepared according to a modified literature procedure (Pyl, T. et al. Justus Liebigs Ann. Chem. 1961, 643 (1), 145-153). To a 2 dram vial was added the α-bromoketone (wherein R′ is as defined for the substituent groups on A in the compounds of Formula I a,d Q′ is C, N, or S, 1 mmol, 1 equiv), 2-aminothiazole (1.3 mmol, 1.3 equiv), and EtOH (1.6 mL) and the reaction mixture was stirred at reflux until disappearance of the α-bromoketone was evident by TLC. The mixture was concentrated, then purified by column chromatography using the given eluent to provide the imidazo[2,1-b]thiazole. For the analogues using 2-amino-4,5-dimethylthiazole, the HCl salt of the heterocycle was commercially available, so NEt₃ (1.3 mmol, 1.3 equiv) was added to the reaction.

Using the above procedures, the following compounds were prepared:

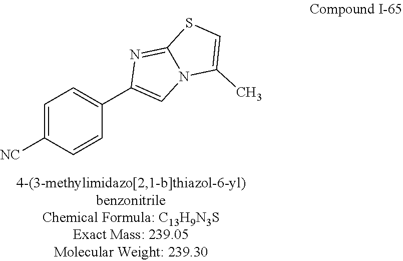 [SMILES: Cc1csc2nc(-c3ccc(C#N)cc3)cn12]

Purified using pentanes-EtOAc (11:9 to 10:10 v:v), triturated with methanol. White solid (26%). ¹H-NMR (CDCl₃, 500 MHz): 7.90 (d, J=8.5 Hz, 2H), 7.69 (s, 1H), 7.64 (d, J=8.5 Hz, 2H), 6.46 (q, J=1.3 Hz, 1H), 2.42 (d, J=1.3 Hz, 3H). ¹³C{¹H}-NMR (CDCl₃, 125 MHz): 150.6, 145.8, 138.8, 132.6, 127.8, 125.5, 119.3, 110.3, 107.8, 107.8, 13.5.

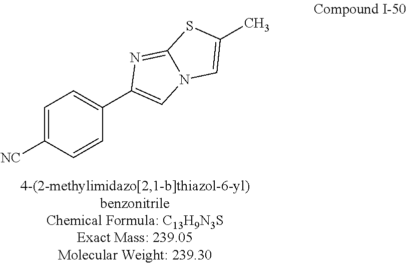 [SMILES: Cc1cn2cc(-c3ccc(C#N)cc3)nc2s1]

Purified using pentanes-EtOAc (12:8 to 8:12 v:v), triturated with methanol. Pale orange solid (30%). ¹H-NMR (CDCl₃, 500 MHz): 7.87 (d, J=8.4 Hz, 2H), 7.70 (s, 1H), 7.64 (d, J=8.4 Hz, 2H), 7.15 (q, J=1.4 Hz, 1H), 2.43 (d, J=1.4 Hz, 3H). ¹³C{¹H}-NMR (CDCl₃, 125 MHz): 150.5, 144.7, 138.8, 132.6, 127.8, 125.4, 119.3, 115.1, 110.2, 109.4, 14.3.

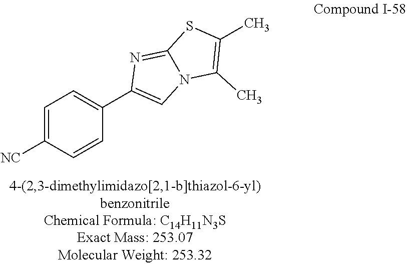 [SMILES: Cc1sc2nc(-c3ccc(C#N)cc3)cn2c1C]

Purified using pentanes-EtOAc (12:8 to 15:5 v:v), triturated with methanol. Pale grey solid (35%). ¹H-NMR (CDCl₃, 500 MHz): 7.9 (d, J=8.1 Hz, 2H), 7.6 (d, J=8.1 Hz, 2H), 7.6 (d, J=1.0 Hz, 1H), 2.3 (s, 3H), 2.3 (s, 3H). ¹³C{¹H}-NMR (CDCl₃, 125 MHz): 148.4, 144.5, 139.0, 132.6, 125.3, 122.8, 120.0, 119.4, 110.1, 107.8, 12.9, 11.1.

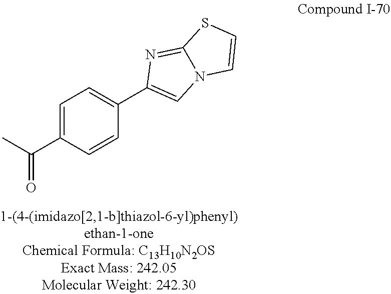 [SMILES: CC(=O)c1ccc(-c2cn3ccsc3n2)cc1]

Purified using pentanes-EtOAc (8:12 to 6:14 v:v), triturated with methanol. White solid (17%, MP=195-198° C.). ¹H-NMR (CDCl₃, 500 MHz): 7.99 (d, J=8.6 Hz, 2H), 7.91 (d, J=8.6 Hz, 2H), 7.84 (s, 1H), 7.45 (d, J=4.2 Hz, 1H), 6.87 (d, J=4.2 Hz, 1H), 2.61 (s, 3H). ¹³C{¹H}-NMR (CDCl₃, 125 MHz): 197.8, 150.8, 146.7, 138.7, 136.0, 129.1, 125.2, 118.6, 113.4, 109.4, 26.7. IR (neat): 3151, 3124, 3082, 1666, 1607, 1553, 1461, 1273, 1198, 847, 748, 724. Mass: ESI+, calc. for C₁₃H₁₁N₂OS 243.0587 [M+H]⁺, found 243.0580.

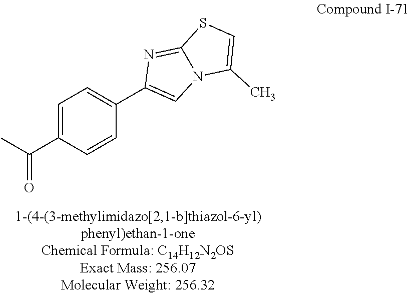 [SMILES: CC(=O)c1ccc(-c2cn3c(C)csc3n2)cc1]

Purified using pentanes-EtOAc (10:10 v:v). Pale yellow solid (17%, MP=158-161° C.). ¹H-NMR (CDCl₃, 400 MHz): 7.99 (d, J=8.6 Hz, 2H), 7.92 (d, J=8.6 Hz, 2H), 7.71 (s, 1H), 6.44 (q, J=1.3 Hz, 1H), 2.61 (s, 3H), 2.43 (d, J=1.3 Hz, 3H). ¹³C{¹H}-NMR (CDCl₃, 125 MHz): 197.8, 150.5, 146.6, 138.9, 135.9, 129.1, 127.8, 125.1, 107.6, 107.5, 26.7, 13.5. IR (neat): 3130, 3002, 2918, 1669, 1604, 1497, 1350, 1267, 1172, 841, 745, 719. Mass: DART+, calc. for C₁₄H₁₃N₂OS 257.0743 [M+H]⁺, found 257.0745.

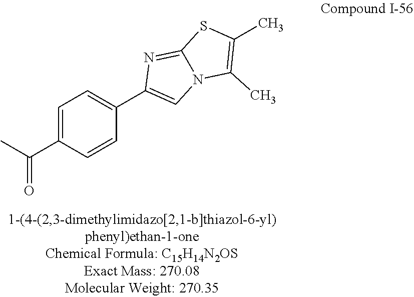 [SMILES: CC(=O)c1ccc(-c2cn3c(C)c(C)sc3n2)cc1]

Purified using pentanes-EtOAc (10:10 v:v), triturated with methanol. Yellow solid (15%, MP=195-198° C.). ¹H-NMR (CDCl₃, 500 MHz): 7.96 (d, J=8.6 Hz, 2H), 7.88 (d, J=8.6 Hz, 2H), 7.59 (s, 1H), 2.59 (s, 3H), 2.32 (d, J=1.1 Hz, 3H), 2.30 (d, J=1.1 Hz, 3H). ¹³C{¹H}-NMR (CDCl₃, 125 MHz): 197.7, 148.1, 145.1, 139.0, 135.6, 129.0, 124.8, 122.8, 119.6, 107.6, 26.7, 12.8, 11.1. IR (neat): 3142, 2969, 2921, 1678, 1604, 1410, 1270, 1184, 844, 719. Mass: DART+, calc. for C₁₅H₁₅N₂OS 271.0900 [M+H]⁺, found 271.0903.

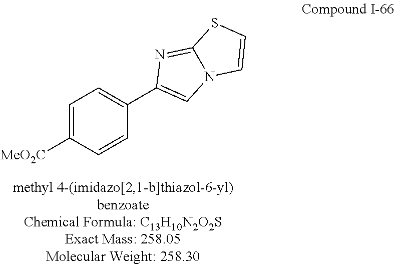 [SMILES: COC(=O)c1ccc(-c2cn3ccsc3n2)cc1]

Purified using pentanes-EtOAc (12:8 to 10:10 v:v), triturated with methanol. White solid (32%). ¹H-NMR (CDCl₃, 500 MHz): 8.06 (d, J=8.5 Hz, 2H), 7.88 (d, J=8.5 Hz, 2H), 7.82 (s, 1H), 7.43 (d, J=4.4 Hz, 1H), 6.85 (d, J=4.4 Hz, 1H), 3.92 (s, 3H). ¹³C{¹H}-NMR (CDCl₃, 125 MHz): 167.1, 150.8, 146.9, 138.6, 130.2, 128.9, 125.0, 118.6, 113.2, 109.3, 52.2.

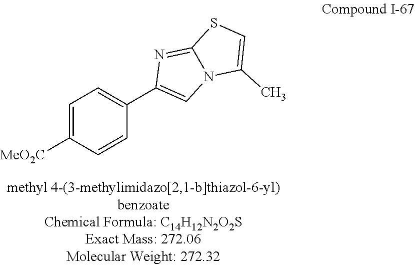 [SMILES: COC(=O)c1ccc(-c2cn3c(C)csc3n2)cc1]

Purified using pentanes-EtOAc (14:6 to 12:8 v:v). Pale yellow solid (33%, MP=145-149° C.). ¹H-NMR (CDCl₃, 500 MHz): 8.05 (d, J=8.8 Hz, 2H), 7.89 (d, J=8.8 Hz, 2H), 7.68 (s, 1H), 6.42 (q, J=1.4 Hz, 1H), 3.91 (s, 3H), 2.41 (d, 1.4 Hz, 3H). ¹³C{¹H}-NMR (CDCl₃, 125 MHz): 167.1, 150.4, 146.7, 138.8, 130.2, 128.7, 127.8, 124.9, 107.4, 107.4, 52.2, 13.5. IR (neat): 3085, 2954, 1723, 1613, 1273, 1192, 1103, 859, 778, 716. Mass: DART+, calc. for C₁₄H₁₃N₂O₂S 273.0692 [M+H]⁺, found 273.0691.

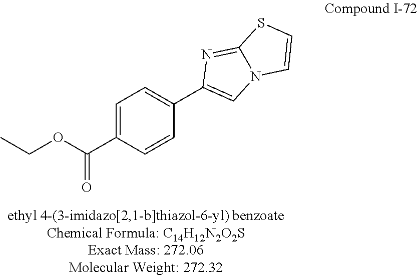 [SMILES: CCOC(=O)c1ccc(-c2cn3ccsc3n2)cc1]

Purified using pentanes-EtOAc (14:6 to 13:7 v:v). White solid (46%). ¹H-NMR (CDCl₃, 400 MHz): 8.06 (d, J=8.7 Hz, 2H), 7.87 (d, J=8.7 Hz, 2H), 7.80 (s, 1H), 7.42 (d, J=4.5 Hz, 1H), 6.83 (d, J=4.5 Hz, 1H), 4.38 (q, J=7.1 Hz, 2H), 1.40 (t, J=7.1 Hz, 3H). ¹³C{¹H}-NMR (CDCl₃, 125 MHz): 166.6, 150.7, 146.8, 138.5, 130.2, 129.2, 124.9, 118.6, 113.2, 109.3, 61.0, 14.5.

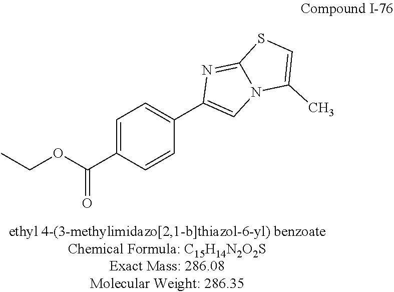 [SMILES: CCOC(=O)c1ccc(-c2cn3c(C)csc3n2)cc1]

Purified using pentanes-EtOAc (14:6 to 13:7 v:v). Pale orange solid (36%, MP=109-113° C.). ¹H-NMR (CDCl₃, 400 MHz): 8.06 (d, J=8.6 Hz, 2H), 7.88 (d, J=8.6 Hz, 2H), 7.68 (s, 1H), 6.41 (q, J=1.3 Hz, 1H), 4.38 (q, J=7.1 Hz, 2H), 2.40 (d, J=1.3 Hz, 3H), 1.40 (t, J=7.1 Hz, 3H). ¹³C{¹H}-NMR (CDCl₃, 125 MHz): 166.6, 150.3, 146.7, 138.6, 130.1, 129.1, 127.8, 124.9, 107.4, 107.4, 61.0, 14.5, 13.5. IR (neat): 3064, 2984, 2915, 1717, 1610, 1470, 1273, 1175, 1112, 864, 778, 721. Mass: DART+, calc. for C₁₅H₁₅N₂O₂S 287.0849 [M+H]⁺, found 287.0857.

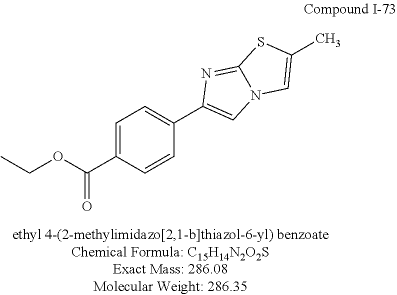 [SMILES: CCOC(=O)c1ccc(-c2cn3cc(C)sc3n2)cc1]

Purified using pentanes-EtOAc (15:5 to 13:7 v:v), triturated with methanol. Pale yellow solid (33%, MP=155-158° C.). ¹H-NMR (CDCl₃, 400 MHz): 8.04 (d, J=8.6 Hz, 2H), 7.84 (d, J=8.6 Hz, 2H), 7.67 (s, 1H), 7.10 (q, J=1.4 Hz, 1H), 4.37 (q, J=7.1 Hz, 2H), 2.40 (d, J=1.4 Hz, 3H), 1.39 (t, J=7.1 Hz, 3H). ¹³C{¹H}-NMR (CDCl₃, 125 MHz): 166.7, 150.2, 145.6, 138.6, 130.1, 128.9, 127.2, 124.7, 115.1, 109.0, 61.0, 14.5, 14.2. IR (neat): 3160, 3064, 2993, 2963, 1705, 1610, 1455, 1267, 1175, 1091, 874, 781, 730. Mass: DART+, calc. for C₁₅H₁₅N₂O₂S 287.0849 [M+H]⁺, found 287.0856.

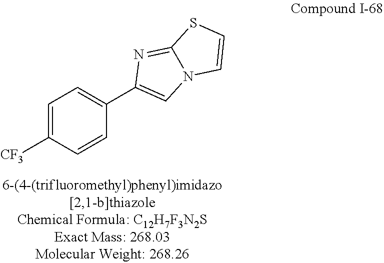 [SMILES: FC(F)(F)c1ccc(-c2cn3ccsc3n2)cc1]

Purified using pentanes-EtOAc (14:6 to 13:7 v:v). Pale yellow solid (42%). ¹H-NMR (CDCl₃, 500 MHz): 7.92 (d, J=8.1 Hz, 2H), 7.79 (s, 1H), 7.64 (d, J=8.1 Hz, 2H), 7.43 (d, J=4.5 Hz, 1H), 6.85 (d, J=4.5 Hz, 1H). ¹³C{¹H}-NMR (CDCl₃, 125 MHz): 150.8, 146.5, 137.7 (q, J=1.4 Hz), 129.2 (q, J=32.4 Hz), 125.8 (q, J=3.9 Hz), 125.4, 124.4 (q, J=271.8 Hz), 118.6, 113.3, 109.1. ¹⁹F{¹H}-NMR (CDCl₃, 375 MHz): -62.4.

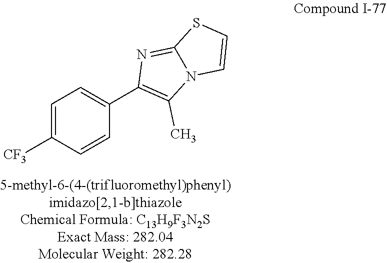 [SMILES: Cc1c(-c2ccc(C(F)(F)F)cc2)nc2sccn12]

Purified using pentanes-EtOAc (15:5 to 13:7 v:v). White solid (31%, MP=158-161° C.). ¹H-NMR (CDCl₃, 400 MHz): 7.83 (d, J=8.1 Hz, 2H), 7.66 (d, J=8.1 Hz, 2H), 7.30 (d, J=4.5 Hz, 1H), 6.85 (d, J=4.5 Hz, 1H), 2.61 (s, 3H). ¹³C{¹H}-NMR (CDCl₃, 125 MHz): 148.0, 141.8, 138.7 (q, J=1.3 Hz), 128.6 (q, J=32.4 Hz), 127.3, 125.5 (q, J=3.8 Hz), 124.5 (d, J=271.9 Hz), 118.8, 116.8, 113.0, 11.0. ¹⁹F{¹H}-NMR (CDCl₃, 375 MHz): -62.3. IR (neat): 3109, 1616, 1547, 1481, 1327, 1100, 1067, 841, 679. Mass: DART+, calc. for C₁₃H₁₀N₂F₃S 283.0511 [M+H]⁺, found 283.0511.

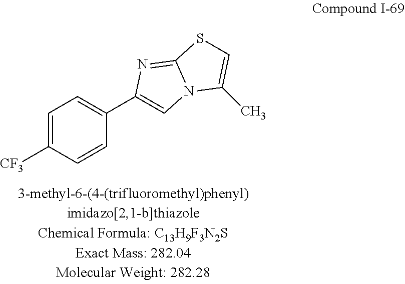 [SMILES: Cc1csc2nc(-c3ccc(C(F)(F)F)cc3)cn12]

Purified using pentanes-EtOAc (15:5 to 14:6 v:v). Orange solid (40%). ¹H-NMR (CDCl₃, 500 MHz): 7.93 (d, J=7.9, 2H), 7.67 (s, 1H), 7.63 (d, J=7.9 Hz, 2H), 6.43 (q, J=1.3 Hz, 1H), 2.42 (d, J=1.3 Hz, 3H). ¹³C{¹H}-NMR (CDCl₃, 125 MHz): 150.4, 146.4, 137.8 (q, J=1.5 Hz), 129.1 (q, J=32.4 Hz), 127.8, 125.8 (q, J=3.8 Hz), 125.3, 124.5 (q, J=271.8 Hz), 107.5, 107.2, 13.5. ¹⁹F{¹H}-NMR (CDCl₃, 375 MHz): -62.4.

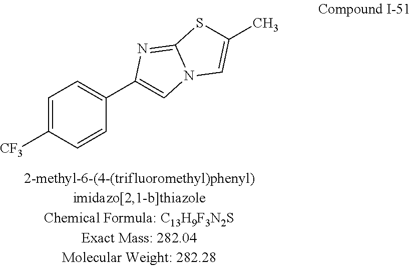 [SMILES: Cc1cn2cc(-c3ccc(C(F)(F)F)cc3)nc2s1]

Purified using pentanes-EtOAc (16:4 to 14:6 v:v), triturated with methanol. White solid (33%, MP=206-210° C.). ¹H-NMR (CDCl₃, 500 MHz): 7.90 (d, J=8.0 Hz, 2H), 7.68 (s, 1H), 7.62 (d, J=8.0 Hz, 2H), 7.15 (q, J=1.4 Hz, 1H), 2.43 (d, J=1.4 Hz, 3H). ¹³C{¹H}-NMR (CDCl₃, 125 MHz): 150.2, 145.0, 137.6, 129.1 (q, J=32.4 Hz), 127.5, 125.8 (q, J=3.9 Hz), 125.2, 124.4 (q, J=271.7 Hz), 115.2, 108.8, 14.2. ¹⁹F{¹H}-NMR (CDCl₃, 375 MHz): -62.4. IR (neat): 3145, 3115, 2936, 1616, 1497, 1321, 1157, 1103, 1067, 856, 722, 692. Mass: DART+, calc. for C₁₃H₉N₂F₃S 283.0511 [M+H]⁺, found 283.0513.

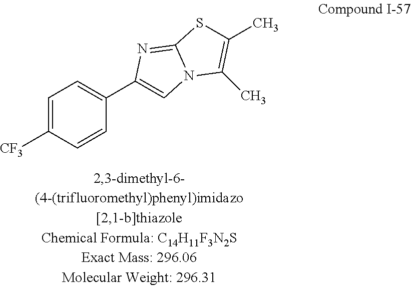 [SMILES: Cc1sc2nc(-c3ccc(C(F)(F)F)cc3)cn2c1C]

Purified using pentanes-EtOAc (16:4 to 15:5 v:v), triturated with methanol. Pale pink solid (18%). ¹H-NMR (CDCl₃, 500 MHz): 7.91 (d, J=8.6 Hz, 2H), 7.62 (d, J=8.6 Hz, 2H), 7.58 (s, 1H), 2.33 (q, J=1.0 Hz, 3H), 2.31 (q, J=1.0 Hz, 3H). ¹³C{¹H}-NMR (CDCl₃, 125 MHz): 148.0, 144.9, 137.8, 128.9 (q, J=32.4 Hz), 125.7 (q, J=3.9 Hz), 125.1, 124.5 (q, J=271.7 Hz), 122.9, 119.7, 107.2, 12.8, 11.1. ¹⁹F{¹H}-NMR (CDCl₃, 375 MHz): -62.4.

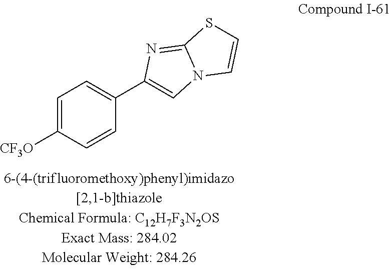

Purified using pentanes-EtOAc (12:8 to 11:9 v:v). Pale yellow solid (44%). ¹H-NMR (CDCl₃, 500 MHz): 7.82 (d, J=8.9 Hz, 2H), 7.71 (s, 1H), 7.40 (d, J=4.5 Hz, 1H), 7.23 (d, J=8.9 Hz, 2H), 6.82 (d, J=4.5 Hz, 1H). ¹³C{¹H}-NMR (CDCl₃, 125 MHz): 150.5, 148.5 (q, J=1.8 Hz), 146.7, 133.1, 126.6, 121.3, 120.6 (q, J=257.0 Hz), 118.6, 112.9, 108.3. ¹⁹F{¹H}-NMR (CDCl₃, 375 MHz): -57.8.

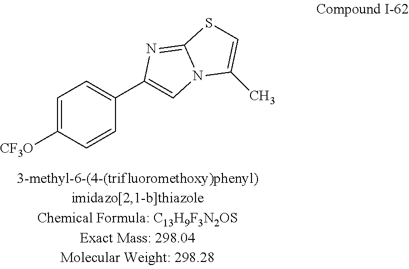

Purified using pentanes-EtOAc (15:5 to 14:6 v:v). Orange solid (43%). ¹H-NMR (CDCl₃, 500 MHz): 7.84 (d, J=8.9 Hz, 2H), 7.59 (s, 1H), 7.23 (dq, J=8.9 Hz, 2H), 6.41 (q, J=1.3 Hz, 1H), 2.41 (d, J=1.3 Hz, 3H). ¹³C{¹H}-NMR (CDCl₃, 125 MHz): 150.2, 148.5 (q, J=1.9 Hz), 146.6, 133.2, 127.8, 126.5, 121.3, 120.7 (q, J=257.0 Hz), 107.1, 106.3, 13.5. ¹⁹F{¹H}-NMR (CDCl₃, 375 MHz): -57.8.

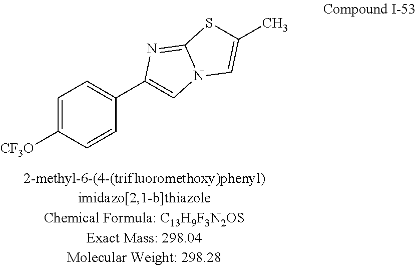

Purified using pentanes-EtOAc (15:5 v:v). Yellow solid (28%, MP=138-143° C.). ¹H-NMR (CDCl₃, 500 MHz): 7.80 (d, J=8.8 Hz, 2H), 7.57 (s, 1H), 7.22 (dd, J=8.8 Hz, 2H), 7.10 (q, J=1.4 Hz, 1H), 2.40 (d, J=1.4 Hz, 3H). ¹³C{¹H}-NMR (CDCl₃, 125 MHz): 150.0, 148.4 (q, J=1.9 Hz), 145.3, 133.1, 126.9, 126.4, 121.3, 120.6 (q, J=256.9 Hz), 115.2, 108.0, 14.2. ¹⁹F{¹H}-NMR (CDCl₃, 375 MHz): -57.8. IR (neat): 3145, 3109, 2939, 1544, 1497, 1258, 1201, 1142, 847, 787, 736, 656. Mass: DART+, calc. for C₁₃H₁₀ON₂F₃S 299.0460 [M+H]⁺, found 299.0458.

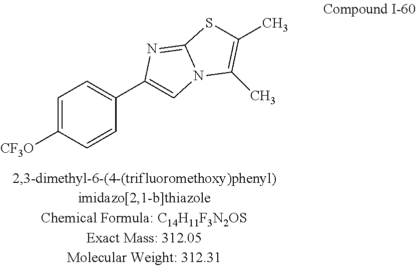

Purified using pentanes-EtOAc (16:4 to 15:5 v:v), triturated with methanol. White solid (13%). ¹H-NMR (CDCl₃, 500 MHz): 7.82 (d, J=8.8 Hz, 2H), 7.49 (s, 1H), 7.22 (d, J=8.8, 2H), 2.32 (q, J=1.0 Hz, 3H), 2.30 (q, J=1.0 Hz, 3H). ¹³C{¹H}-NMR (CDCl₃, 125 MHz): 148.3 (q, J=1.8 Hz), 147.8, 145.2, 133.4, 126.3, 122.8, 121.3, 120.7 (q, J=256.8 Hz), 119.2, 106.3, 12.8, 11.1. ¹⁹F{¹H}-NMR (CDCl₃, 375 MHz): -57.8.

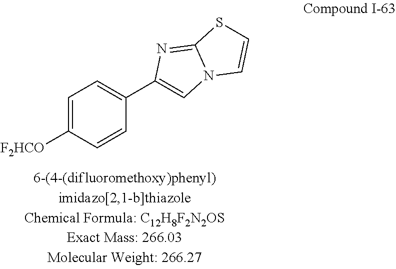 [SMILES: FC(F)Oc1ccc(-c2cn3ccsc3n2)cc1]

Purified using pentanes-EtOAc (13:7 to 11:9 v:v). White solid (41%). ¹H-NMR (CDCl₃, 400 MHz): 7.80 (d, J=8.9 Hz, 2H), 7.69 (s, 1H), 7.40 (d, J=4.5 Hz, 1H), 7.14 (d, J=8.9 Hz, 2H), 6.81 (d, J=4.5 Hz, 1H), 6.53 (t, J=74.1 Hz, 1H). ¹³C{¹H}-NMR (CDCl₃, 125 MHz): 150.6 (t, J=2.8 Hz), 150.4, 146.9, 131.7, 126.7, 119.8, 118.6, 116.1 (t, J=259.4 Hz), 112.7, 108.0. ¹⁹F{¹H}-NMR (CDCl₃, 375 MHz): -80.5.

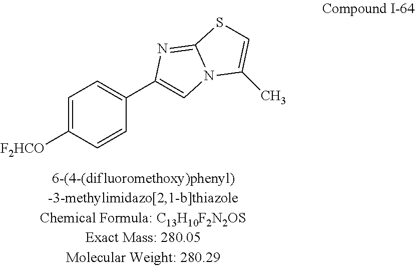 [SMILES: Cc1csc2nc(-c3ccc(OC(F)F)cc3)cn12]

Purified using pentanes-EtOAc (14:6 v:v). Orange solid (41%). ¹H-NMR (CDCl₃, 400 MHz): 7.81 (d, J=8.8 Hz, 2H), 7.57 (s, 1H), 7.13 (d, J=8.8 Hz, 2H), 6.53 (t, J=74.1 Hz, 1H), 6.39 (q, J=1.3 Hz, 1H), 2.40 (d, J=1.3 Hz, 3H). ¹³C{¹H}-NMR (CDCl₃, 125 MHz): 150.5 (t, J=2.9 Hz), 150.0, 146.8, 131.9, 127.8, 126.6, 119.8, 116.1 (t, J=259.3 Hz), 106.9, 106.1, 13.5. ¹⁹F{¹H}-NMR (CDCl₃, 375 MHz): -80.5.

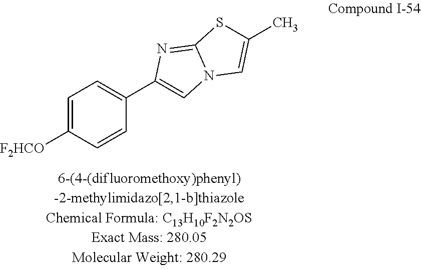 [SMILES: Cc1cn2cc(-c3ccc(OC(F)F)cc3)nc2s1]

Purified using pentanes-EtOAc (14:6 to 13:7 v:v). Yellow solid (32%, MP=125-129° C.). ¹H-NMR (CDCl₃, 500 MHz): 7.78 (d, J=8.8 Hz, 2H), 7.57 (s, 1H), 7.15-7.10 (m, 3H), 6.52 (t, J=74.1 Hz, 1H), 2.41 (d, J=1.4 Hz, 3H). ¹³C{¹H}-NMR (CDCl₃, 125 MHz): 150.5 (t, J=2.8 Hz), 149.9, 145.5, 131.7, 126.9, 126.5, 119.8, 116.2 (t, J=259.3 Hz), 115.2, 107.8, 14.2. ¹⁹F{¹H}-NMR (CDCl₃, 375 MHz): -80.5. IR (neat): 3145, 3109, 2987, 2939, 1547, 1470, 1380, 1222, 1121, 1031, 847, 739. Mass: DART+, calc. for C₁₃H₁₁ON₂F₂S 281.0555 [M+H]⁺, found 281.0545.

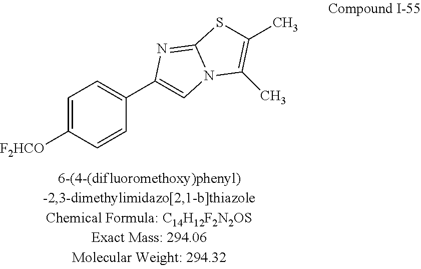 [SMILES: Cc1sc2nc(-c3ccc(OC(F)F)cc3)cn2c1C]

Purified using pentanes-EtOAc (15:5 to 14:6 v:v). Yellow solid (52%). ¹H-NMR (CDCl₃, 500 MHz): 7.80 (d, J=8.8 Hz, 2H), 7.48 (s, 1H), 7.12 (d, J=8.8 Hz, 2H), 6.52 (t, J=74.1 Hz, 1H), 2.33 (q, J=0.9 Hz, 3H), 2.30 (q, J=0.9 Hz, 3H). ¹³C{¹H}-NMR (CDCl₃, 125 MHz): 150.4 (t, J=2.8 Hz), 147.7, 145.3, 131.9, 126.5, 122.9, 119.8, 119.1, 116.2 (t, J=259.2 Hz), 106.1, 12.8, 11.1. ¹⁹F{¹H}-NMR (CDCl₃, 375 MHz): -80.5.

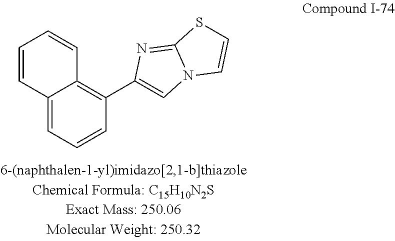 [SMILES: c1ccc2c(-c3cn4ccsc4n3)cccc2c1]

Purified using pentanes-EtOAc (13:7 v:v). Brown oil (42%). ¹H-NMR (CDCl₃, 400 MHz): 8.63-8.58 (m, 1H), 7.91-7.88 (m, 1H), 7.85 (dt, J=8.3, 1.2 Hz, 1H), 7.75 (dd, J=7.1, 1.3 Hz, 1H), 7.70 (s, 1H), 7.55-7.48 (m, 3H), 7.45 (d, J=4.5 Hz, 1H), 6.83 (d, J=4.5 Hz, 1H). ¹³C{¹H}-NMR (CDCl₃, 125 MHz): 149.8, 147.2, 134.1, 132.0, 131.4, 128.4, 128.3, 127.2, 126.4, 126.0, 125.9, 125.5, 118.6, 112.6, 111.2.

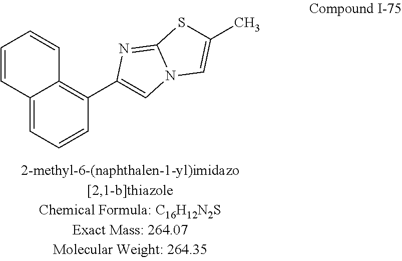 [SMILES: Cc1cn2cc(-c3cccc4ccccc34)nc2s1]

Purified using pentanes-EtOAc (15:5 to 14:6 v:v). Brown oil (33%). ¹H-NMR (CDCl₃, 400 MHz): 8.63-8.57 (m, 1H), 7.91-7.86 (m, 1H), 7.83 (dt, J=8.3, 1.2 Hz, 1H), 7.74 (dd, J=7.1, 1.3 Hz, 1H), 7.59 (s, 1H), 7.55-7.46 (m, 3H), 7.17 (q, J=1.4 Hz, 1H), 2.44 (d, J=1.4 Hz, 3H). ¹³C{¹H}-NMR (CDCl₃, 125 MHz): 149.3, 145.8, 134.1, 132.0, 131.4, 128.4, 128.2, 127.1, 126.6, 126.4, 126.1, 125.8, 125.5, 115.2, 110.9, 14.2. IR (neat): 3106, 3052, 2924, 1592, 1466, 1252, 1207, 804, 778, 730, 667. Mass: DART+, calc. for C₁₆H₁₃N₂S 265.0794 [M+H]⁺, found 265.0795.

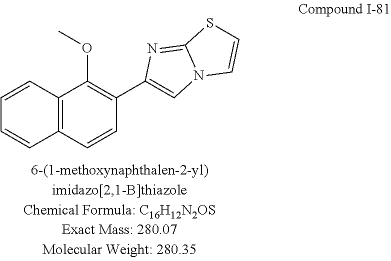 [SMILES: COc1c(-c2cn3ccsc3n2)ccc2ccccc12]

Purified using DCM-MeOH (99:1 to 98:2 v:v). Orange solid (31%, MP=130-133° C.). ¹H-NMR (CDCl₃, 500 MHz): 8.35 (d, J=8.7 Hz, 1H), 8.24 (s, 1H), 8.18-8.15 (m, 1H), 7.86 (ddd, J=8.0, 1.3, 0.7 Hz, 1H), 7.72 (dd, J=8.6, 0.7 Hz, 1H), 7.53 (ddd, J=8.3, 6.8, 1.3 Hz, 1H), 7.49-7.45 (m, 2H), 6.84 (dd, J=4.4, 0.5 Hz, 1H), 3.92 (s, 3H). ¹³C{¹H}-NMR (CDCl₃, 125 MHz): 152.4, 149.4, 143.5, 134.5, 128.4, 128.2, 126.2, 126.1, 126.0, 124.5, 122.9, 122.4, 118.7, 112.7, 112.6, 60.5. IR (neat): 3118, 2936, 2850, 1559, 1464, 1371, 1198, 1073, 987, 831, 760, 712. Mass: DART+, calc. for C₁₆H₁₃N₂OS 281.0743 [M+H]⁺, found 281.0739.

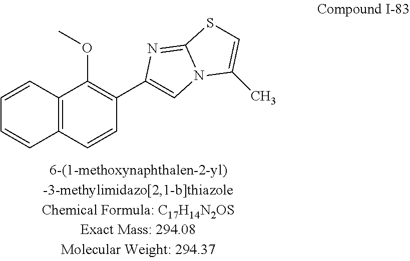 [SMILES: COc1c(-c2cn3c(C)csc3n2)ccc2ccccc12]

Purified using pentanes-EtOAc (15:5 to 14:6 v:v). Brown oil (13%). ¹H-NMR (CDCl₃, 400 MHz): 8.36 (d, J=8.8 Hz, 1H), 8.17 (ddt, J=8.4, 1.4, 0.8 Hz, 1H), 8.13 (s, 1H), 7.86 (d, J=8.1 Hz, 1H), 7.72 (dd, J=8.8, 0.8 Hz, 1H), 7.53 (ddd, J=8.4, 6.8, 1.4 Hz, 1H), 7.47 (ddd, J=8.1, 6.8, 1.4 Hz, 1H), 6.43 (q, J=1.3 Hz, 1H), 3.93 (s, 3H), 2.46 (d, J=1.3 Hz, 3H). ¹³C{¹H}-NMR (CDCl₃, 125 MHz): 152.4, 149.0, 143.3, 134.4, 128.4, 128.2, 127.9, 126.2, 126.0, 124.5, 123.0, 122.3, 110.7, 107.0, 60.6, 13.6. IR (neat): 3115, 3061, 2933, 2847, 1473, 1371, 1276, 1186, 1073, 987, 823, 751, 661. Mass: DART+, calc. for C₁₇H₁₅N₂OS 295.0900 [M+H]⁺, found 295.0899.

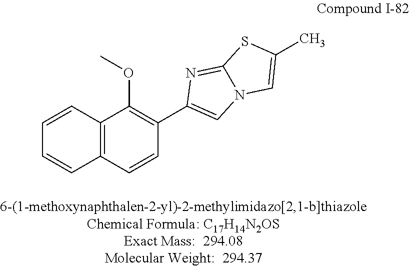 [SMILES: COc1c(-c2cn3cc(C)sc3n2)ccc2ccccc12]

Purified using DCM-MeOH (99:1 to 98:2 v:v). White solid (31%, MP=165-168° C.). ¹H-NMR (CDCl₃, 500 MHz): 8.34 (d, J=8.7 Hz, 1H), 8.15 (dd, J=8.3, 1.3 Hz, 1H), 8.11 (s, 1H), 7.85 (ddd, J=8.1, 1.3, 0.7 Hz, 1H), 7.70 (dd, J=8.7, 0.7 Hz, 1H), 7.52 (ddd, J=8.3, 6.8, 1.3 Hz, 1H), 7.46 (ddd, J=8.1, 6.8, 1.3 Hz, 1H), 7.17 (q, J=1.4 Hz, 1H), 3.90 (s, 3H), 2.42 (d, J=1.4 Hz, 3H). ¹³C{¹H}-NMR (CDCl₃, 125 MHz): 152.2, 148.9, 142.1, 134.3, 128.4, 128.2, 126.6, 126.1, 126.1, 125.9, 124.4, 123.0, 122.3, 115.3, 112.4, 60.4, 14.2. IR (neat): 3169, 2924, 2852, 1476, 1371, 1213, 1070, 987, 822, 763. Mass: DART+, calc. for C₁₇H₁₄N₂OS 295.0900 [M+H]⁺, found 295.0896.

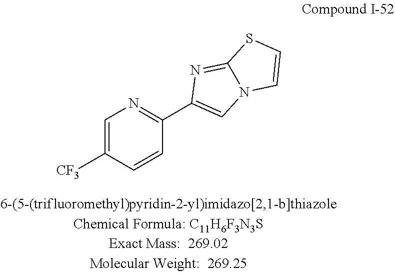 [SMILES: FC(F)(F)c1ccc(-c2cn3ccsc3n2)nc1]

Purified using pentanes-EtOAc (12:8 to 10:10 v:v), triturated with methanol. Pale orange solid (25%, 176-180° C. sublimates). ¹H-NMR (CDCl₃, 500 MHz): 8.80 (dd, J=2.5, 0.9 Hz, 1H), 8.21 (s, 1H), 8.14 (dd, J=8.3, 0.9 Hz, 1H), 7.96 (dd, J=8.3, 2.5 Hz, 1H), 7.49 (d, J=4.5 Hz, 1H), 6.91 (d, J=4.5 Hz, 1H). ¹³C{¹H}-NMR (CDCl₃, 125 MHz): 156.3, 150.8, 146.7, 146.4 (q, J=4.2 Hz), 134.1 (q, J=3.6 Hz), 124.7 (q, J=33.0 Hz), 123.9 (q, J=271.8 Hz), 119.3, 118.8, 114.0, 112.4. ¹⁹F{¹H}-NMR (CDCl₃, 375 MHz): -62.2. IR (neat): 3174, 3141, 3109, 3067, 1613, 1529, 1461, 1321, 1148, 1106, 1061, 865, 730, 673. Mass: DART+, calc. for C₁₁H₇N₃F₃S 270.0307 [M+H]⁺, found 270.0306.

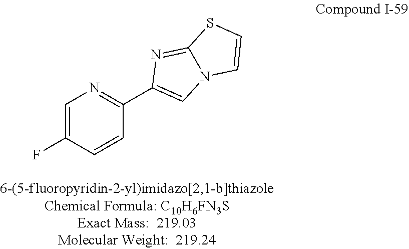 [SMILES: Fc1ccc(-c2cn3ccsc3n2)nc1]

Purified using DCM-MeOH (99:1 v:v). Orange solid (35%). ¹H-NMR (CDCl₃, 500 MHz): 8.40 (dd, J=2.9, 0.6 Hz, 1H), 8.03-7.99 (m, 2H), 7.45-7.41 (m, 2H), 6.84 (d, J=4.5 Hz, 1H). ¹³C{¹H}-NMR (CDCl₃, 125 MHz): 158.7 (d, J=255.1 Hz), 150.3, 149.5 (d, J=3.8 Hz), 147.1, 137.5 (d, J=24.0 Hz), 123.6 (d, J=18.5 Hz), 120.7 (d, J=4.4 Hz), 118.7, 113.2, 110.6. ¹⁹F{¹H}-NMR (CDCl₃, 375 MHz): -129.1.

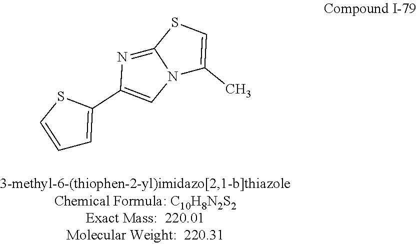

Purified using pentanes-EtOAc (15:5 to 14:6 v:v). Orange solid (35%). ¹H-NMR (CDCl₃, 400 MHz): 7.51 (s, 1H), 7.33 (dd, J=3.5, 1.2 Hz, 1H), 7.22 (dd, J=5.1, 1.2 Hz, 1H), 7.04 (dd, J=5.1, 3.5 Hz, 1H), 6.38 (q, J=1.3 Hz, 1H), 2.38 (d, J=1.3 Hz, 3H). ¹³C{¹H}-NMR (CDCl₃, 125 MHz): 149.8, 142.7, 138.0, 127.7, 127.7, 124.1, 122.6, 107.0, 105.6, 13.5.

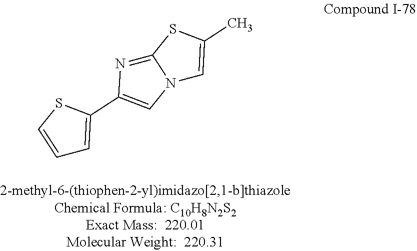

Purified using pentanes-EtOAc (14:6 v:v). Pale orange solid (37%). ¹H-NMR (CDCl₃, 400 MHz): 7.50 (s, 1H), 7.29 (dd, J=3.6, 1.1 Hz, 1H), 7.20 (dd, J=5.1, 1.1 Hz, 1H), 7.07 (q, J=1.4 Hz, 1H), 7.03 (dd, J=5.1, 3.6 Hz, 1H), 2.38 (d, J=1.4 Hz, 3H). ¹³C{¹H}-NMR (CDCl₃, 125 MHz): 149.6, 141.6, 138.0, 127.7, 126.7, 123.8, 122.3, 115.1, 107.2, 14.1.

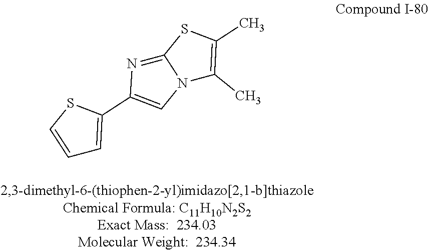

Purified using pentanes-EtOAc (16:4 to 15:5 v:v). Orange solid (49%). ¹H-NMR (CDCl₃, 400 MHz): 7.41 (s, 1H), 7.30 (dd, J=3.5, 1.2 Hz, 1H), 7.19 (dd, J=5.1, 1.2 Hz, 1H), 7.03 (dd, J=5.1, 3.5 Hz, 1H), 2.30 (q, J=1.0 Hz, 3H), 2.28 (q, J=1.0 Hz, 3H). ¹³C{¹H}-NMR (CDCl₃, 125 MHz): 147.4, 141.3, 138.2, 127.7, 123.7, 122.8, 122.2, 119.0, 105.6, 12.7, 11.1.

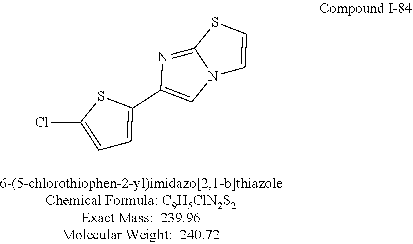 [SMILES: Clc1ccc(-c2cn3ccsc3n2)s1]

Purified using pentanes-EtOAc (13:7 to 12:8 v:v). Orange solid (46%). ¹H-NMR (CDCl₃, 500 MHz): 7.57 (s, 1H), 7.38 (dd, J=4.5, 0.5 Hz, 1H), 7.06 (d, J=3.9 Hz, 1H), 6.84 (dd, J=3.9, 0.5 Hz, 1H), 6.81 (dd, J=4.5, 0.5 Hz, 1H). ¹³C{¹H}-NMR (CDCl₃, 125 MHz): 150.3, 142.0, 136.5, 128.6, 126.8, 121.6, 118.5, 113.0, 107.4.

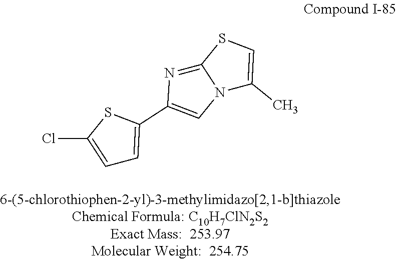

Purified using pentanes-EtOAc (14:6 to 13:7 v:v). Orange solid (35%). ¹H-NMR (CDCl₃, 500 MHz): 7.45 (s, 1H), 7.07 (d, J=3.8 Hz, 1H), 6.84 (d, J=3.8 Hz, 1H), 6.40 (q, J=1.4 Hz, 1H), 2.39 (d, J=1.4 Hz, 3H). ¹³C{¹H}-NMR (CDCl₃, 125 MHz): 149.9, 141.9, 136.7, 128.4, 127.8, 126.8, 121.5, 107.2, 105.5 13.5.

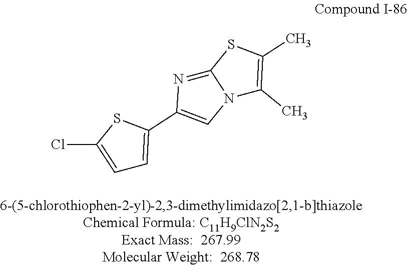 [SMILES: Cc1sc2nc(-c3ccc(Cl)s3)cn2c1C]

Purified using pentanes-EtOAc (14:6 v:v). Orange solid (49%). ¹H-NMR (CDCl₃, 500 MHz): 7.35 (s, 1H), 7.03 (d, J=3.9 Hz, 1H), 6.82 (d, J=3.9 Hz, 1H), 2.31 (q, J=1.1 Hz, 3H), 2.27 (q, J=1.1 Hz, 3H). ¹³C{¹H}-NMR (CDCl₃, 125 MHz): 147.6, 140.6, 137.0, 128.0, 126.7, 122.8, 121.1, 119.3, 105.5, 12.8, 11.1.

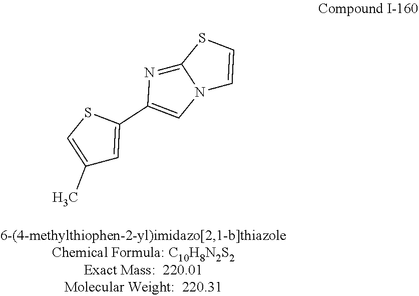

Purified using pentanes-EtOAc (14:6 to 12:8 v:v). Pale red solid (34%, MP=94-98° C.). ¹H-NMR (CDCl₃, 500 MHz): 7.59 (s, 1H), 7.36 (d, J=4.5 Hz, 1H), 7.14 (d, J=1.2 Hz, 1H), 6.80 (p, J=1.2 Hz, 1H), 6.78 (d, J=4.5 Hz, 1H), 2.26 (d, J=1.2 Hz, 3H). ¹³C{¹H}-NMR (CDCl₃, 125 MHz): 150.1, 143.0, 138.3, 137.4, 125.0, 119.5, 118.5, 112.6, 107.3, 15.9. IR (neat): 3114, 3076, 2924, 1553, 1461, 1243, 1181, 897, 834, 721. Mass: DART+, calc. for C₁₀H₉N₂S₂ 221.0202 [M+H]⁺, found 221.0203.

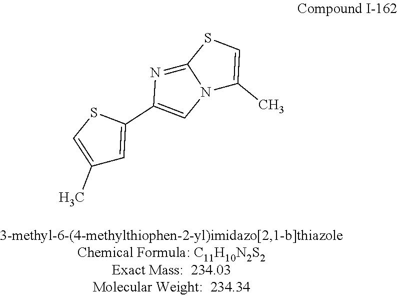

Purified using pentanes-EtOAc (15:5 to 13:7 v:v). Orange solid (40%, MP=104-108° C.). ¹H-NMR (CDCl₃, 500 MHz): 7.47 (s, 1H), 7.16 (d, J=1.1 Hz, 1H), 6.79 (p, J=1.1 Hz, 1H), 6.37 (q, J=1.3 Hz, 1H), 2.38 (d, J=1.3 Hz, 3H), 2.27 (d, J=1.1 Hz, 3H). ¹³C{¹H}-NMR (CDCl₃, 125 MHz): 149.7, 142.9, 138.3, 137.6, 127.7, 125.0, 119.4, 106.9, 105.4, 15.9, 13.5. IR (neat): 3100, 2915, 1469, 1434, 1276, 1175, 1043, 891, 825, 728. Mass: DART+, calc. for C₁₁H₁₁N₂S₂ 235.0358 [M+H]⁺, found 235.0352.

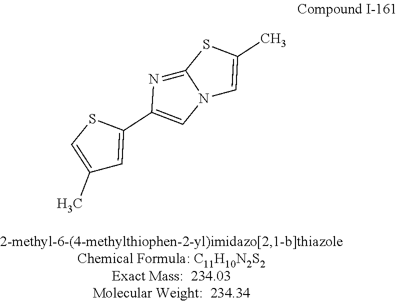

Purified using pentanes-EtOAc (15:5 to 13:7 v:v). Orange solid (39%, MP=123-127° C.). ¹H-NMR (CDCl₃, 500 MHz): 7.46 (s, 1H), 7.11 (d, J=1.1 Hz, 1H), 7.06 (q, J=1.4 Hz, 1H), 6.77 (p, J=1.1 Hz, 1H), 2.38 (d, J=1.4 Hz, 3H), 2.26 (d, J=1.1 Hz, 3H). ¹³C{¹H}-NMR (CDCl₃, 125 MHz): 149.5, 141.7, 138.2, 137.7, 126.6, 124.7, 119.2, 115.1, 107.0, 15.9, 14.1. IR (neat): 3148, 3100, 2918, 1466, 1261, 1183, 897, 844, 784, 733. Mass: DART+, calc. for C₁₁H₁₁N₂S2 235.0358 [M+H]⁺, found 235.0356.

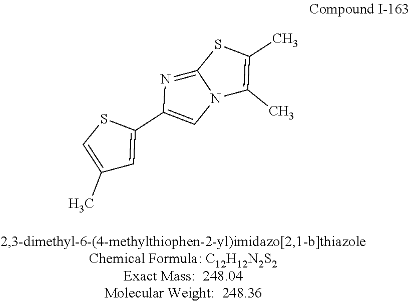 [SMILES: Cc1csc(-c2cn3c(C)c(C)sc3n2)c1]

Purified using pentanes-EtOAc (17:3 to 15:5 v:v). Orange solid (49%, MP=143-147° C.). ¹H-NMR (CDCl₃, 500 MHz): 7.38 (s, 1H), 7.13 (d, J=1.2 Hz, 1H), 6.77 (p, J=1.2 Hz, 1H), 2.30 (q, J=1.0 Hz, 3H), 2.28 (q, J=1.0 Hz, 3H), 2.26 (d, J=1.2 Hz, 3H). ¹³C{¹H}-NMR (CDCl₃, 125 MHz): 147.4, 141.5, 138.2, 137.9, 124.6, 122.8, 119.0, 118.9, 105.4, 15.9, 12.7, 11.1. IR (neat): 3157, 3094, 3064, 2918,1467,1255,1180,888,822,766,707. Mass: DART+, calc. for C₁₂H₁₃N₂S₂ 249.0515 [M+H]⁺, found 249.0513.

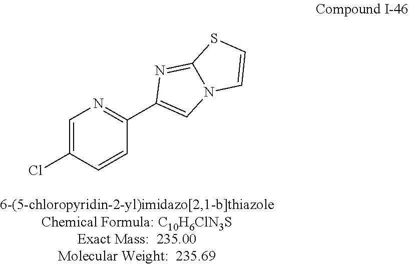 [SMILES: Clc1ccc(-c2cn3ccsc3n2)nc1]

### Biological Assays

C. elegans Dose-Response Experiments

Forty microliters of an HB101 E. coli bacterial suspension in liquid NGM (nematode growth media—see ref. 42 for the recipe) was added to each well of a 96-well flat-bottom culture plate, after which approximately 25 synchronized L1 worms, in 10 μl of M9 buffer (see ref. 43 for the recipe), were added to each well. The synchronized L1 worms were obtained from an embryo preparation performed the previous day (see ref. 43 for the protocol). For the L1 assays, 0.5 μl of test compound solution (or DMSO alone) was immediately added to the wells using a multichannel pipette; the final DMSO concentration is 1% (v/v). The worms were incubated for 3 days at 20° C. and the number of viable animals was counted. A dead worm was considered any worm that failed to move after vigorous agitation of the plate, and that appeared morphologically “dead”, i.e. clear appearance and unresolved internal structures.

At least three biological replicates were performed for each dose-response assay. For each biological replicate, two technical replicates were performed and the numbers of viable animals for each technical replicate were combined (i.e. ˜ 50 worms assayed per concentration). The number of viable worms at each concentration was divided by the corresponding DMSO control value to give the “relative viability” for each concentration. The “relative viability” values were then averaged across the biological replicates. Minimum lethal concentration was defined as the lowest concentration at which at least 20% of worms die.

Danio rerio (Zebrafish) Culture and Dose-Response Experiments

Zebrafish were maintained and handled under the guidance and approval of the Canadian Council on Animal Care and the Hospital for Sick Children Laboratory Animal Services. Dose-response assays were performed using zebrafish (Danio rerio) at 3 days post-fertilization. Three wild-type (TLAB) embryos were analyzed in each well of a 24-well plate containing 1 mL of E3 medium (5 mM NaCl, 0.17 mM KCl, 0.33 mM CaCl₂, 0.33 mM MgSO₄). Test compounds were added to each well to reach final concentrations of 45 μM, 15 μM, and 5 μM (0.5% v/v DMSO), as well as a DMSO control well. The viability of the fish was assessed after 3 days of test compound exposure using a Zeiss standard dissection microscope. The LC₁₀₀ was defined as the lowest concentration tested that resulted in all of the fish dying.

### HepG2 Cell Proliferation Assay

HepG2 cells, which are liver-derived, were counted using a haemocytometer, diluted, and seeded in 384-well plates to a final density of 5×10⁴ cells/mL in 100 uL of RPMI-1640 medium supplemented with 10% heat inactivated fetal bovine serum (Gibco) and 1.2× Antibiotic-Antimycotic (Gibco). Cells were incubated at 37° C. with 5% CO₂ for 24 hours. Subsequently, a 2-fold dilution series of test compound was added to cells at a final volume of 200 uL and incubated at 37° C. with 5% CO₂ for 72 hours. After 72 hours, Alamar Blue (Invitrogen) was added to the Hep G2 cells at a final concentration of 0.5× and plates were incubated at 37° C. for 4 hours. Fluorescence was measured at Ex560 nm/Em590 nm and corrected for background from the medium. All assays were performed in technical triplicates and in at least two biological replicates. The IC₅₀ value was defined as the concentration that inhibits cell proliferation by 50% of the untreated control cells.

Meloidogyne incognita In Vitro Larval Mobility Assays

M. incognita (Kofoid & White) Chitwood Race 1 (originally isolated in Maryland) was used for all experiments, and was maintained on pepper (Capsicum annuum L.) cv. PA-136 in a greenhouse as previously described⁴⁴. Infective J2 juveniles were collected as described in ref. 45. The microwell dose-response experiments were carried out similarly to previously described protocols^{44,46}. In brief, 50 μL of deionized water was added to the wells of 96-well polystyrene plates and 0.5 μL of test compound (or DMSO alone) was added to each well. Approximately 35 J2s were then added to each well in 49.5 μL of deionized water. The chemicals were tested at 45 μM, and the final concentration of DMSO in each well was 0.5% (v/v). DMSO alone was added to 3 separate wells. The plates were covered with plastic adhesive strips, and the lids of the plates were sealed with parafilm. The plates were incubated at 25° C. without shaking for 4 days. The percent of worms that were immobile (% immobile) was quantified by counting the number of immobile worms after 4 days of incubation, dividing the number of immobile worms by the total number of worms in the well, and then multiplying by 100. Two replicates were performed for each treatment. The z-score for each treatment was calculated using the mean and standard deviation of the untreated DMSO control wells. The average % immobile value was calculated across the two replicates. A test compound was considered most promising if the z-score was greater than 1.645 in both replicates.

### Meloidogyne Chitwoodi In Vitro Larval Mobility Assays

M. chitwoodi race 1 (the strain commonly found in the pacific northwest of the United States) was used for all experiments, and was maintained on tomato plants (Solanum lycopersicum ‘Rutgers’) as previously described⁴⁷. The microwell dose-response experiments were carried out similarly to previously described protocols^{44,46}. In brief, 100 J2s, in 10 μL of deionized water, were added to the wells of 96-well polystyrene plates, after which 190 μL of deionized water containing dissolved test compound, or DMSO alone, was added to each well. The chemicals were tested at 45 μM, and the final concentration of DMSO in each well was 0.5% (v/v). The wells were covered with a plastic adhesive strip, and the lids of the plates were sealed with parafilm. The plates were incubated at 25° C. for 2 days. The percent of worms that were immobile (% immobile) was quantified by counting the number of immobile worms after 2 days of incubation, dividing the number of immobile worms by the total number of worms in the well, and then multiplying by 100. Three replicates were performed for each chemical treatment, and for the untreated DMSO controls as well. The average % immobile value was calculated across the three replicates. A chemical was considered most promising if a student's t-test comparing the mean of the treatment with the mean of the untreated controls resulted in a p-value less than 1.0×10⁻².

### Meloidogyne Hapla In Vitro Egg Hatch Assays

The M. hapla were originally obtained from a soil sample of muck soil from Ste-Clotilde, Quebec, Canada. Using tweezers, egg masses were carefully removed from root material from infested carrot roots. Eggs were liberated from the egg masses using a mild sodium hypochlorite bleaching protocol, whereby egg masses were bleached with a 10% commercial bleach (sodium hypochlorite) solution for 4 minutes with vigorous shaking, and then washed 5 times with deionized water. 50 μL of deionized water was added to each well of two 96-well polystyrene culture plates, and 0.5 μL of chemical (or DMSO alone) was added to each well. Approximately 90 eggs were added to each well in 49.5 ul of deionized water. The plates were sealed with parafilm and incubated for 8 days at room temperature without shaking in a Tupperware box filled with wet paper towels. After the 8-day incubation, the number of hatched eggs was counted for each treatment and for the untreated controls. Each chemical treatment was tested only once. 8 separate untreated DMSO controls were included in the experiment. The z-score for each treatment was calculated using the mean and standard deviation of the untreated DMSO control wells. A chemical was considered to robustly inhibit egg hatching if its Z-score was greater than 3

Meloidogyne incognita Infectivity Assays

An M. incognita population originally collected from grape (Vitis vinifera) in Parlier, California, was used for all experiments, and they were maintained on tomato plants (Solanum lycopersicum ‘Rutgers’) as previously described⁴⁷. Infective J2 juveniles were collected as described in ref. 47.

For the infectivity assays, 90 grams of soil (1:1 sand:loam mix) was added to each cell of several 6-cell plastic garden packs. The soil was drenched with 18 mL of deionized water containing dissolved test compound or DMSO alone. 2,500 infective J2 juveniles were then added to the soil in 2 mL of deionized water, for a total water volume of 20 mL. The final concentration of the test compound in waterways 45 μM. The DMSO concentration varied from 0.1% to 0.8% (v/v) depending on the stock concentration of the test compound. The highest DMSO concentration was used as the DMSO control. The J2s were incubated in the soil and chemical for 24 hours, after which two- to three-week old tomato seedlings were transplanted into the soil (one plant per cell). Two replicates were performed for each chemical treatment, and four replicates were done for the DMSO controls. The whole experiment was replicated twice, in two different batches on two different days, for a total of four replicates for each test compound treatment, and eight replicates for the DMSO controls. Inoculated plants were grown for 8 weeks in a greenhouse, as described⁴⁷, under long-day conditions (16-h photoperiod) with 26/18° C. day/night temperatures. After 8 weeks, the plants were destructively harvested. The tops were removed and discarded, and roots were gently washed with water to remove adhering soil. Eggs were extracted by placing rinsed roots in 0.6% sodium hypochlorite and agitating at 300 rpm for 3 min. Roots were then rinsed over nested 250—and 25.4-μm sieves, with eggs collected from the latter and suspended in water. Roots were dried in a 65° C. oven for at least 24 hours, after which dry roots were weighed. The number of eggs from each plant root was counted on a dissection microscope using a haemocytometer, and the number of eggs per milligram of root was calculated by dividing the total egg number by the mass of the dried root material. An average was taken across the replicates performed on the same day, and then normalized to the DMSO control average. To calculate percent effectiveness at inhibiting reproduction, the normalized values were subtracted from 1, and then multiplied by 100. An average percent effectiveness value was then calculated across the two different batches carried out on different days.

### Meloidogyne Chitwoodi Infectivity Assays

M. chitwoodi race 1 (the strain commonly found in the pacific northwest of the United States) was used for all experiments, and was maintained on tomato plants (Solanum lycopersicum ‘Rutgers’) as previously described⁴⁷. The M. chitwoodi infectivity assays were performed identically to the M. incognita infectivity assays (see above), with the exception that egg counts were not normalized to the mass of the roots. Four technical replicates were performed in a single batch. An average was taken across the four replicates performed on the same day, and then normalized to the DMSO control average. To calculate percent effectiveness at inhibiting reproduction, the normalized values were subtracted from 1, and then multiplied by 100.

### Results

Compounds of Formula I Demonstrate Nematicidal Activity Against the Free-Living Nematode Caenorhabditis elegans

C. elegans is a small, free-living, and easy-to-culture nematode that permits the facile assessment of nematode killing by small molecules. As a first approach to test the nematicidal potential of compounds 1-1 to 1-47, dose-response assays were performed with C. elegans. Compound 1-18 was not available at the time of testing, and was therefore omitted from this analysis. 27 of the compounds killed C. elegans with minimum lethal concentrations less than or equal to 100 μM (Table 1). Compounds 1-10, 1-31, and 1-32 were the most potently lethal nematicides in this assay, having minimum lethal concentrations of 6.25 μM. The remaining compounds were relatively inactive against C. elegans. These results suggest that compounds of Formula I can potently kill nematodes.

TABLE 1
Effects of compounds of Formula I on the viability of C. elegans.

| Compound | Minimum Lethal | Minimum Lethal |
|---|---|---|
| No. | Concentration (μM) | Concentration (ppm) |
| I-1 | >100 | >21.4 |
| I-2 | 50 | 14.8 |
| I-3 | >100 | >22.8 |
| I-4 | 100 | 22.8 |
| I-5 | 25 | 5.7 |
| I-6 | >100 | >22.8 |
| I-7 | 50 | 11.4 |
| I-8 | 50 | 12.1 |
| I-9 | 100 | 24.2 |
| I-10 | 6.25 | 1.5 |
| I-11 | 100 | 22.8 |
| I-12 | 100 | 25.6 |
| I-13 | >100 | >23.2 |
| I-14 | 100 | 21.8 |
| I-15 | >100 | >23.2 |
| I-16 | >100 | >23.2 |
| I-17 | 100 | 23.2 |
| I-18 | n.d. | n.d. |
| I-19 | >100 | >23.2 |
| I-20 | >100 | >23.2 |
| I-21 | 50 | 11.6 |
| I-22 | 12.5 | 3.1 |
| I-23 | 100 | 23.5 |
| I-24 | 100 | 24.9 |
| I-25 | >100 | >24.9 |
| I-26 | 25 | 6.2 |
| I-27 | 100 | 23.5 |
| I-28 | >100 | >24.9 |
| I-29 | 100 | 24.9 |
| I-30 | >100 | >24.9 |
| I-31 | 6.25 | 1.7 |
| I-32 | 6.25 | 1.7 |
| I-33 | 12.5 | 3.7 |
| I-34 | 50 | 14.0 |
| I-35 | >50 | 14.7 |
| I-36 | >100 | >29.3 |
| I-37 | >100 | >29.3 |
| I-38 | 100 | 27.9 |
| I-39 | >100 | >29.3 |
| I-40 | >100 | >29.3 |
| I-41 | >100 | >29.3 |
| I-42 | 25 | 8.2 |
| I-43 | 50 | 16.3 |
| I-44 | >100 | >21.5 |
| I-45 | >100 | >20.1 |
| I-46 | 100 | 23.6 |
| I-47 | 50 | 10.9 |
| tioxazafen | 3.125 | 0.7 |

n.d. not determined

Compounds of Formula I Inhibit the Movement of Infective Larvae from the Plant-Parasitic Root-Knot Nematode Species Meloidgyne Incognita and Meloidogyne Chitwoodi In Vitro

Encouraged by their nematicidal activity against C. elegans, next the activity of compounds of Formula I against plant parasitic nematodes (PPNs) was assessed. To that end, in vitro experiments were performed to test the effects of an expanded set of compounds of Formula I on the movement of infective J2 larvae from the plant-parasitic root-knot nematode species M. incognita. The infective J2 larvae were treated with 45 μM (˜10 ppm) of the test compounds for 4 days, and the percentage of nematodes that were immobile for each treatment was assessed. This was performed in duplicate. Three untreated DMSO controls were also performed for each replicate. The commercial nematicides tioxazafen, fluopyram, abamectin, fenamiphos, oxamyl, and iprodione were used as positive controls. A compound having a percent immobile count greater than 1.645 standard deviations above the mean for the untreated samples (i.e. a Z-score>1.645) in both replicates was most promising. Based on these criteria a false positive would be obtained at a relatively low rate of 1 in every 400 untreated samples. Of the 155 compounds of Formula I tested, 44 were found to have a Z-score>1.645 (Table 2). Compounds 1-14, 1-23, 1-34, 1-46, 1-47, 1-49, 1-63, 1-84, 1-89, 1-93, 1-113, 1-122, 1-128, 1-152, 1-154, 1-157, and 1-159 resulted in at least 75% immobility. With the exception of iprodione, all of the positive control nematicides were active.

TABLE 2
Effects of compounds of Formula I on the movement of M. incognita J2 larvae in vitro.

| | | | Replicate 1 | | Replicate 2 | | |
|---|---|---|---|---|---|---|---|
| Compound | Concentration | Concentration | % Immobile | | % Immobile | | Average |
| Name | (μM) | (ppm) | [z-score] | | [z-score] | | % Immobile^{a} |
| I-2 | 45 | 13.3 | 93.85 | [14.54] | 43.10 | [6.03] | 68.47 |
| I-3 | 45 | 10.3 | 56.67 | [6.94] | 25.00 | [2.76] | 40.83 |
| I-6 | 45 | 10.3 | 43.48 | [4.25] | 27.59 | [3.22] | 35.53 |
| I-7 | 45 | 10.3 | 74.47 | [10.58] | 29.41 | [3.55] | 51.94 |
| I-13 | 45 | 10.5 | 35.29 | [2.58] | 21.57 | [2.14] | 28.43 |
| I-14 | 45 | 9.8 | 98.21 | [15.43] | 82.22 | [13.11] | 90.22 |
| I-21 | 45 | 10.5 | 50.98 | [5.78] | 40.82 | [5.62] | 45.90 |
| I-22 | 45 | 11.2 | 59.65 | [7.55] | 33.33 | [4.26] | 46.49 |
| I-23 | 45 | 10.6 | 94.44 | [14.66] | 85.00 | [13.61] | 89.72 |
| I-24 | 45 | 11.2 | 30.91 | [1.68] | 36.21 | [4.78] | 33.56 |
| I-26 | 45 | 11.2 | 54.35 | [6.47] | 20.37 | [1.92] | 37.36 |
| I-27 | 45 | 10.6 | 56.00 | [6.81] | 27.91 | [3.28] | 41.95 |
| I-30 | 45 | 11.2 | 63.49 | [8.34] | 40.82 | [5.62] | 52.15 |
| I-31 | 45 | 12.1 | 34.09 | [2.33] | 36.17 | [4.78] | 35.13 |
| I-33 | 45 | 13.2 | 31.37 | [1.78] | 32.76 | [4.16] | 32.07 |
| I-34 | 45 | 12.6 | 96.08 | [15.00] | 87.76 | [14.11] | 91.92 |
| I-41 | 45 | 13.2 | 38.89 | [3.31] | 46.34 | [6.62] | 42.62 |
| I-42 | 45 | 14.7 | 51.52 | [5.89] | 70.73 | [11.03] | 61.12 |
| I-45 | 45 | 9.1 | 32.08 | [1.92] | 27.45 | [3.20] | 29.76 |
| I-46 | 45 | 10.6 | 73.24 | [10.33] | 77.08 | [12.18] | 75.16 |
| I-47 | 45 | 9.8 | 95.92 | [14.96] | 95.56 | [15.52] | 95.74 |
| I-48 | 45 | 12.7 | 100.00 | [15.80] | 38.00 | [5.11] | 69.00 |
| I-49 | 45 | 10.7 | 100.00 | [15.80] | 100.00 | [16.33] | 100.00 |
| I-53 | 45 | 13.4 | 38.98 | [3.33] | 47.27 | [6.79] | 43.13 |
| I-54 | 45 | 12.6 | 31.15 | [1.73] | 21.67 | [2.15] | 26.41 |
| I-61 | 45 | 12.8 | 66.04 | [8.86] | 48.15 | [6.94] | 57.09 |
| I-63 | 45 | 12.0 | 100.00 | [15.80] | 100.00 | [16.33] | 100.00 |
| I-84 | 45 | 10.8 | 89.06 | [13.56] | 100.00 | [16.33] | 94.53 |
| I-87 | 45 | 10.1 | 56.86 | [6.98] | 63.27 | [9.68] | 60.06 |
| I-89 | 45 | 9.8 | 74.65 | [10.62] | 77.59 | [12.27] | 76.12 |
| I-93 | 45 | 11.8 | 100.00 | [15.80] | 76.92 | [12.15] | 88.46 |
| I-113 | 45 | 13.4 | 100.00 | [15.80] | 94.44 | [15.32] | 97.22 |
| I-116 | 45 | 9.1 | 44.23 | [4.40] | 23.21 | [2.43] | 33.72 |
| I-122 | 45 | 10.3 | 95.92 | [14.96] | 77.78 | [12.31] | 86.85 |
| I-128 | 45 | 11.3 | 81.03 | [11.92] | 85.45 | [13.69] | 83.24 |
| I-129 | 45 | 11.6 | 33.33 | [2.18] | 19.61 | [1.78] | 26.47 |
| I-141 | 45 | 11.8 | 31.91 | [1.89] | 32.20 | [4.06] | 32.06 |
| I-142 | 45 | 10.4 | 36.17 | [2.76] | 19.05 | [1.68] | 27.61 |
| I-144 | 45 | 12.6 | 43.10 | [4.17] | 47.62 | [6.85] | 45.36 |
| I-152 | 45 | 10.9 | 100.00 | [15.80] | 91.30 | [14.75] | 95.65 |
| I-154 | 45 | 12.3 | 73.58 | [10.40] | 100.00 | [16.33] | 86.79 |
| I-157 | 45 | 11.1 | 98.04 | [15.40] | 94.00 | [15.24] | 96.02 |
| I-158 | 45 | 11.2 | 37.93 | [3.12] | 56.82 | [8.51] | 47.37 |
| I-159 | 45 | 12.6 | 97.78 | [15.34] | 100.00 | [16.33] | 98.89 |
| Tioxazafen | 45 | 10.3 | 100.00 | [14.82] | 100.00 | [21.99] | 100.00 |
| Fluopyram | 45 | 17.9 | 100.00 | [14.82] | 100.00 | [21.99] | 100.00 |
| Abamectin | 45 | 39.3 | 94.34 | [13.88] | 89.47 | [19.43] | 91.91 |
| Fenamiphos | 45 | 13.7 | 100.00 | [14.82] | 100.00 | [21.99] | 100.00 |
| Oxamyl | 45 | 9.9 | 67.16 | [9.35] | 66.67 | [13.88] | 66.92 |
| Iprodione | 45 | 14.9 | 10.91 | [−0.02] | 16.67 | [1.71] | 13.79 |
| Untreated 1 | — | — | 21.28 | [1.71] | 7.02 | [−0.63] | 14.15 |
| Untreated 2 | — | — | 18.64 | [1.27] | 6.15 | [−0.84] | 12.40 |
| Untreated 3 | — | — | 28.13 | [2.85] | 16.13 | [1.58] | 22.13 |

^{a}% immobile values are the average of the two experimental replicates.

The in vitro effects of a smaller subset of compounds of formula I on the movement of J2 larvae from the root-knot nematode species M. chitwoodi, after 2 days of exposure at 45 μM (˜10 ppm) was also tested. Both the treatment and untreated DMSO control conditions were performed in triplicate. The commercial nematicides tioxazafen and fluopyram were used as positive controls. Of the eight compounds tested, treatment with compounds I-2, 1-7, 1-10, 1-13, 1-22, 1-33, 1-46, and 1-47 resulted in average % immobility values that are statistically significantly different from the untreated control average (i.e. student's t-test p-value<1.0×10⁻²) (Table 3). As expected, treatment with both positive control nematicides resulted in average % immobility values that are statistically significantly different from the untreated control average (Table 3). Compounds 1-2, 1-7, 1-10, and 1-22 resulted in % immobility greater than 75%. Together with the M. incognita data, these results demonstrate that compounds of formula I are effective at inhibiting the movement of PPNs at low parts per million values.

TABLE 3
Effects of compounds of Formula I on the movement
of M. chitwoodi J2 larvae in vitro.

| Compound | Concentration | Concentration | | |
|---|---|---|---|---|
| Name | (μM) | (ppm) | % Immobile^{a} | p-value^{b} |
| I-1 | 45 | 9.6 | 48.2 | 1.4 × 10⁻² |
| I-2 | 45 | 13.3 | 95.8 | 3.9 × 10⁻⁶ |
| I-7 | 45 | 10.3 | 78.8 | 6.6 × 10⁻⁴ |
| I-10 | 45 | 10.9 | 100 | 2.2 × 10⁻⁸ |
| I-13 | 45 | 10.5 | 32.4 | 1.9 × 10⁻³ |
| I-22 | 45 | 11.2 | 75.3 | 8.3 × 10⁻⁵ |
| I-33 | 45 | 13.2 | 58.1 | 1.8 × 10⁻³ |
| I-45 | 45 | 9.1 | 13.8 | 0.7 × 10⁰ |
| I-46 | 45 | 10.6 | 56.4 | 1.0 × 10⁻³ |
| I-47 | 45 | 9.8 | 62.7 | 1.7 × 10⁻⁵ |
| tioxazafen | 45 | 10.3 | 42.7 | 6.2 × 10⁻³ |
| fluopyram | 45 | 17.9 | 100 | 2.2 × 10⁻⁸ |
| untreated | — | — | 12.6 | 1 |

^{b}p-value was determined using student's t-test comparing the mean of the treatment group versus the mean of the untreated group.

Compounds of Formula I can Inhibit Egg-Hatching of the Root-Knot Nematode Meloidogyne hapla

In addition to the movement assays described above, the effects of the expanded set of compounds of Formula I on the hatching of M. hapla eggs was tested. Eggs were liberated from root egg masses using a mild bleaching procedure. Eggs were treated with 45 μM (˜10 ppm) of each test compound, and after 8 days at room temperature the number of hatched eggs was quantified. Eight untreated DMSO controls were also included in the test. The commercial nematicides tioxazafen, fluopyram, abamectin, fenamiphos, oxamyl, and iprodione were used as positive controls. This experiment was performed only once. A compound having a number-of-hatched-eggs value more than 3 standard deviations below the mean for the untreated samples (i.e. a Z-score>3) as most promising. Based on these criteria a false positive would be obtained at a relatively low rate of 1 in every 740 untreated samples. Of the 159 compounds tested, 44 were to have a Z-score>3 (Table 4). Compounds 1-48, 1-84, 1-154, and 1-161 completely inhibited egg hatching. With the exception of oxamyl and iprodione, all of the positive control nematicides had a Z-score>3 in this assay. These results suggest that in addition to inhibiting the movement of infective larvae, the compounds of Formula I can inhibit egg hatching of PPNs.

TABLE 4
Effects of compounds of Formula I on the
hatching of M. hapla eggs in vitro.

| | | | Number of | |
|---|---|---|---|---|
| Compound | Concentration | Concentration | Hatched Eggs | |
| Name | (μM) | (ppm) | [z-score] | |
| I-4 | 45 | 10.3 | 4 | [3.31] |
| I-7 | 45 | 10.3 | 1 | [4.30] |
| I-9 | 45 | 10.9 | 2 | [3.97] |
| I-11 | 45 | 10.3 | 3 | [3.64] |
| I-14 | 45 | 9.8 | 1 | [4.30] |
| I-17 | 45 | 10.5 | 3 | [3.64] |
| I-22 | 45 | 11.2 | 1 | [4.30] |
| I-23 | 45 | 10.6 | 1 | [4.30] |
| I-25 | 45 | 11.2 | 4 | [3.31] |
| I-27 | 45 | 10.6 | 4 | [3.31] |
| I-29 | 45 | 11.2 | 4 | [3.31] |
| I-31 | 45 | 12.1 | 1 | [4.30] |
| I-34 | 45 | 12.6 | 1 | [4.30] |
| I-35 | 45 | 13.2 | 2 | [3.97] |
| I-43 | 45 | 14.7 | 4 | [3.31] |
| I-47 | 45 | 9.8 | 1 | [4.30] |
| I-48 | 45 | 12.7 | 0 | [4.63] |
| I-49 | 45 | 10.7 | 2 | [3.97] |
| I-61 | 45 | 12.8 | 1 | [4.30] |
| I-62 | 45 | 13.4 | 3 | [3.64] |
| I-63 | 45 | 12.0 | 2 | [3.97] |
| I-64 | 45 | 12.6 | 4 | [3.31] |
| I-68 | 45 | 12.1 | 4 | [3.31] |
| I-72 | 45 | 12.3 | 4 | [3.31] |
| I-78 | 45 | 9.9 | 3 | [3.64] |
| I-81 | 45 | 12.6 | 3 | [3.64] |
| I-84 | 45 | 10.8 | 0 | [4.63] |
| I-85 | 45 | 11.5 | 4 | [3.31] |
| I-86 | 45 | 12.1 | 4 | [3.31] |
| I-87 | 45 | 10.1 | 3 | [3.64] |
| I-89 | 45 | 9.8 | 3 | [3.64] |
| I-94 | 45 | 12.6 | 4 | [3.31] |
| I-101 | 45 | 11.3 | 2 | [3.97] |
| I-108 | 45 | 12.4 | 4 | [3.31] |
| I-109 | 45 | 12.3 | 4 | [3.31] |
| I-116 | 45 | 9.1 | 2 | [3.97] |
| I-118 | 45 | 13.2 | 2 | [3.97] |
| I-122 | 45 | 10.3 | 1 | [4.30] |
| I-132 | 45 | 10.3 | 3 | [3.64] |
| I-135 | 45 | 12.3 | 4 | [3.31] |
| I-144 | 45 | 12.6 | 4 | [3.31] |
| I-153 | 45 | 11.0 | 2 | [3.97] |
| I-154 | 45 | 12.3 | 0 | [4.63] |
| I-161 | 45 | 10.5 | 0 | [4.63] |
| Tioxazafen | 45 | 10.3 | 1 | [4.30] |
| Fluopyram | 45 | 17.9 | 0 | [4.63] |
| Abamectin | 45 | 39.3 | 1 | [4.30] |
| Fenamiphos | 45 | 13.7 | 2 | [3.97] |
| Oxamyl | 45 | 9.9 | 9 | [1.65] |
| Iprodione | 45 | 14.9 | 15 | [−0.33] |
| Untreated 1 | — | — | 13 | [0.33] |
| Untreated 2 | — | — | 9 | [1.65] |
| Untreated 3 | — | — | 14 | [0.00] |
| Untreated 4 | — | — | 11 | [0.99] |
| Untreated 5 | — | — | 16 | [−0.66] |
| Untreated 6 | — | — | 18 | [−1.32] |
| Untreated 7 | — | — | 17 | [0.00] |
| Untreated 8 | — | — | 14 | [0.00] |

Compounds of Formula I can Inhibit the Infection of Tomato Plant Roots by the Plant-Parasitic Nematodes Meloidogyne incognita and Meloidogyne Chitwoodi

The inhibition of movement observed with compounds of Formula I in the in vitro assay was promising, however it is good to confirm that compounds that are active in vitro do not lose activity in soil-based experiments. The loss of activity that occurs when transitioning from in vitro assays to soil-based experiments could be a result of the compounds adsorbing onto the various components of the soil mixture, thereby reducing their aqueous concentration. The converse is also true; compounds that do not obviously inhibit the movement of nematodes in vitro can sometimes prevent root infection in soil-based experiments. Commercially useful nematicides ideally prevent the infection of plant roots in the soil. Thus, to assess their “real-world” potential, 25 compounds of Formula I were tested for their ability to prevent root infection of tomato plants in soil (Table 5). 24 of the 25 compounds were tested against M. incognita, and 2 out of 25 compounds were tested against M. chitwoodi. Before planting, the test compounds were diluted in water and then added to the soil, after which infective J2 larvae were added to the soil in water. The final concentration for all of the compounds was 45 μM (˜10 ppm). The nematodes were incubated in the test compounds in soil for 24 hours, after which tomato seedlings were planted. The nematodes were given 8 weeks to infect the roots and produce eggs, after which the number of eggs per unit mass of roots was calculated. The percent effectiveness at inhibiting nematode reproduction in the roots, relative to the DMSO control, was then calculated for each compound. This value is used as a proxy to assess the infectivity of the nematodes. 18 out of 25 compounds tested against M. incognita reduced nematode reproduction in the roots to a level below that of the untreated samples (Table 5). Compound 1-46 has a percent effectiveness (37.7%) similar to that of the commercial nematicide tioxazafen (43.7%), used here as a positive control. The two compounds tested against M. chitwoodi, 1-7 and 1-10, were 59.3% and 63.7% effective at inhibiting reproduction, respectively, relative to the untreated samples (Table 5). A positive control was not included alongside the M. chitwoodi experiments. These results show that compounds of Formula I, at low parts per million concentrations, can inhibit plant root infection by parasitic nematodes in the soil, and support the real-world utility of these compounds as nematicidal agents. Furthermore, treatment of tomato plants with compounds of formula I did not reduce root weights relative to the DMSO control, suggesting that these compounds do not have obvious phytotoxic effects on root growth (Table 6).

TABLE 5
Effects of compounds of Formula I on the reproduction of root-knot nematodes in roots.

| | | | % effectiveness at | % effectiveness at |
|---|---|---|---|---|
| Compound | Concentration | Concentration | inhibiting reproduction | inhibiting reproduction |
| Name | (μM) | (ppm) | of M. incognita | of M. chitwoodi |
| I-7 | 45 | 10.3 | n.d. | 59.3 |
| I-10 | 45 | 10.9 | 18.5 | 63.7 |
| I-13 | 45 | 10.5 | 0.0 | n.d. |
| I-14 | 45 | 9.8 | 17.6 | n.d. |
| I-15 | 45 | 10.5 | 5.8 | n.d. |
| I-16 | 45 | 10.5 | 0.0 | n.d. |
| I-17 | 45 | 10.5 | 21.8 | n.d. |
| I-18 | 45 | 9.8 | 0.0 | n.d. |
| I-19 | 45 | 10.5 | 16.6 | n.d. |
| I-21 | 45 | 10.5 | 24.4 | n.d. |
| I-24 | 45 | 11.2 | 23.6 | n.d. |
| I-25 | 45 | 11.2 | 24.6 | n.d. |
| I-26 | 45 | 11.2 | 0.0 | n.d. |
| I-27 | 45 | 10.6 | 0.0 | n.d. |
| I-28 | 45 | 11.2 | 17.4 | n.d. |
| I-29 | 45 | 11.2 | 15.0 | n.d. |
| I-30 | 45 | 11.2 | 16.0 | n.d. |
| I-31 | 45 | 12.1 | 18.1 | n.d. |
| I-32 | 45 | 12.1 | 4.4 | n.d. |
| I-33 | 45 | 13.2 | 7.9 | n.d. |
| I-35 | 45 | 13.2 | 10.2 | n.d. |
| I-37 | 45 | 13.2 | 21.6 | n.d. |
| I-38 | 45 | 12.6 | 0.0 | n.d. |
| I-41 | 45 | 13.2 | 6.6 | n.d. |
| I-46 | 45 | 10.6 | 37.7 | n.d. |
| tioxazafen | 45 | 10.3 | 43.7 | n.d. |

n.d. not determined

TABLE 6
Effects of compounds of Formula I on the root mass of tomato plants.

| Compound | Concentration | Concentration | Normalized root mass |
|---|---|---|---|
| Name | (μM) | (ppm) | (relative to DMSO control) |
| I-10 | 45 | 10.9 | 1.30 |
| I-13 | 45 | 10.5 | 1.12 |
| I-14 | 45 | 9.8 | 1.28 |
| I-15 | 45 | 10.5 | 1.35 |
| I-16 | 45 | 10.5 | 1.15 |
| I-17 | 45 | 10.5 | 1.30 |
| I-18 | 45 | 9.8 | 1.13 |
| I-19 | 45 | 10.5 | 1.27 |
| I-21 | 45 | 10.5 | 1.24 |
| I-24 | 45 | 11.2 | 1.34 |
| I-25 | 45 | 11.2 | 1.27 |
| I-26 | 45 | 11.2 | 1.43 |
| I-27 | 45 | 10.6 | 1.42 |
| I-28 | 45 | 11.2 | 1.37 |
| I-29 | 45 | 11.2 | 1.14 |
| I-30 | 45 | 11.2 | 1.30 |
| I-31 | 45 | 12.1 | 1.25 |
| I-32 | 45 | 12.1 | 1.31 |
| I-33 | 45 | 13.2 | 1.45 |
| I-35 | 45 | 13.2 | 1.26 |
| I-37 | 45 | 13.2 | 1.31 |
| I-38 | 45 | 12.6 | 1.21 |
| I-41 | 45 | 13.2 | 1.18 |
| I-46 | 45 | 10.6 | 1.56 |
| tioxazafen | 45 | 10.3 | 1.09 |

### Compounds of Formula I are Selectively Active Against PPNs

In order to replace the commercial nematicides that are being phased out due to unfavourable ecotoxicity, newly discovered nematicides ideally demonstrate selectivity for parasitic nematodes relative to non-target species such as fish and humans. In addition, recently marketed next-generation nematicides, such as fluensulfone and fluazaindolizine, are selective for PPNs over nematodes that do not parasitize plants, many of which can be beneficial to the soil^{32,33,48,49}. To test the selectivity of compound 1-46 for PPNs its activity was assessed in human HepG2 cells, zebrafish embryos, and the free-living nematode C. elegans. Compound 1-46 was selected for these experiments because it is the most robustly active of all of the compounds tested in the soil-based infectivity assays (Table 5). Similar to the commercial nematicide tioxazafen, compound 1-46 is relatively inactive against human HepG2 cells, with an IC₅₀ greater than 100 μM (˜23 ppm) (Table 7). Compound 1-46 is non-lethal to zebrafish at concentrations up to 45 μM, which is the highest concentration tested, and so its LC₅₀ in this organism is greater than 45 μM (Table 7). In contrast, tioxazafen has an LC₅₀ value in zebrafish of at most 15 μM, suggesting that it is at least 3 times more potent at killing fish than compound 1-46 (Table 7). Compound 1-46 is also relatively inactive against the free-living nematode C. elegans, with a minimum lethal concentration of 100 μM (Table 7). In comparison, tioxazafen kills C. elegans at concentrations as low as 3.125 μM (Table 7), suggesting that it is 32 times more potent at killing C. elegans than compound 1-46. Altogether, these results suggest that compounds of Formula I can be similarly effective as commercial nematicides against PPNs in soil-based infection assays, but have selectivity for parasitic nematodes that is comparable to, or better than, commercially used compounds.

TABLE 7
Bioactivity summary for compound I-46 of Formula
I and the commercial nematicide tioxazafen.

| | | | | % effectiveness at |
|---|---|---|---|---|
| Compound | HepG2 | Zebrafish | C. elegans | inhibiting reproduction |
| Name | IC₅₀ (μM)^{a, b} | LC₁₀₀ (μM)^{c} | MLC (μM)^{d} | of M. incognita at 45 μM |
| I-46 | >100 | >45 | 100 | 37.7 |
| tioxazafen | >100 | 15 | 3.125 | 43.7 |

^{a}HepG2 cells are derived from human liver.
^{b}IC₅₀ is the concentration at which HepG2 cell proliferation is inhibited to 50% of untreated control cells.
^{c}LC₁₀₀ is the lowest concentration at which all of the fish are dead.
^{d}MLC is the minimum lethal concentration (see Materials and Methods for a more complete definition).

### FULL CITATIONS FOR DOCUMENTS REFERRED TO IN THE APPLICATION

(1) United Nations, Department of Economic and Social Affairs, and Population Division. (2017) World Population Prospects: The 2017 Revision, Key Findings and Advance Tables.
(2) Alexandratos, N., and Bruinsma, J. (2012) World agriculture towards 2030/2050: the 2012 revision.
(3) Godfray, H. C. J., and Garnett, T. (2014) Food security and sustainable intensification. Philos. Trans. R. Soc. B 369, 20120273.
(4) Popp, J., Peto, K., and Nagy, J. (2013) Pesticide productivity and food security. A review. Agron. Sustain. Dev. 33, 243-255.
(5) Foley, J. A., Ramankutty, N., Brauman, K. A., Cassidy, E. S., Gerber, J. S., Johnston, M., Mueller, N. D., Connell, C. O., Ray, D. K., West, P. C., Balzer, C., Bennett, E. M., Sheehan, J., Siebert, S., Carpenter, S. R., Hill, J., Monfreda, C., Polasky, S., Rockstro, J., Tilman, D., and Zaks, D. P. M. (2011) Solutions for a cultivated planet. Nature 478, 337-342.
(6) Foley, J. A., Defries, R., Asner, G. P., Barford, C., Bonan, G., Carpenter, S. R., Chapin, F. S., Coe, M. T., Daily, G. C., Gibbs, H. K., Helkowski, J. H., Holloway, T., Howard, E. A., Kucharik, C. J., Monfreda, C., Patz, J. A., Prentice, I. C., Ramankutty, N., and Snyder, P. K. (2005) R EVIEW Global Consequences of Land Use. Science (80-.). 309, 570-574.
(7) Cunningham, S. A., Attwood, S. J., Bawa, K. S., Benton, T. G., Broadhurst, L. M., Didham, 25 R. K., Mcintyre, S., Perfecto, I., Samways, M. J., Tscharntke, T., Vandermeer, J., Villard, M., Young, A. G., and Lindenmayer, D. B. (2013) To close the yield-gap while saving biodiversity will require multiple locally relevant strategies. Agric. Ecosyst. Environ. 173, 20-27.
(8) Hunter, M. C., Smith, R. G., Schipanski, M. E., Atwood, L. W., and Mortensen, D. A. (2017) Agriculture in 2050: Recalibrating Targets for Sustainable Intensification. Bioscience 67, 386-391.
(9) Tilman, D., Balzer, C., Hill, J., and Befort, B. L. (2011) Global food demand and the sustainable intensification of agriculture. Proc. Natl. Acad. Sci. 108, 20260-20264.
(10) Oerke, E.-C. (2006) Crop losses to pests. J. Agric. Sci. 144, 31-43.
(11) Grisi, L., Leite, R. C., de Souza Martins, J. R., de Barros, A. T. M., Andreotti, R., de Leon, A. A. P., Pereira, J. B., and Villela, H. S. (2014) Reassessment of the potential economic impact of cattle parasites in Brazil. Brazilian J. Vet. Parasitol. 23, 150-156.
(12) Trudgill, D. L., and Blok, V. C. (2001) APOMICTIC, POLYPHAGOUS ROOT-KNOT NEMATODES: Exceptionally Successful and Damaging Biotrophic Root Pathogens. Annu. Rev. Phytopathol. 39, 53-77.
(13) Nicol, J. M., Turner, S. J., Coyne, D. L., den Nijs, L., Hockland, S., and Tahna Maafi, Z. (2011) Current Nematode Threats to World Agriculture, in Genomics and Molecular Genetics of Plant-Nematode Interactions (Jones, J., Gheysen, G., and Fenoll, C., Eds.), pp 21-43. Springer Science+Business Media.
(14) Jones, J. T., Haegeman, A., Danchin, E. G. J., Gaur, H. S., Helder, J., Jones, M. G. K., Kikuchi, T., Manzanilla-L6pez, R., Palomares-Rius, J. E., Wesemael, W. M. L., and Perry, R. N. (2013) Top 10 plant-parasitic nematodes in molecular plant pathology. Mol. Plant Pathol. 14, 946-961.
(15) Bernard, G. C., Egnin, M., and Bonsi, C. (2017) The Impact of Plant-Parasitic Nematodes on Agriculture and Methods of Control, in Nematology—Concepts, Diagnosis, and Control (Shah, M. M., and Mahamood, M., Eds.), pp 121-138. InTech.
(16) Githigia, S. M., Thamsborg, S. M., Munyua, W. K., and Maingi, N. (2001) Impact of gastrointestinal helminths on production in goats in Kenya. Small Rumin. Res. 42, 21-29.
(17) Mavrot, F., Hertzberg, H., and Torgerson, P. (2015) Effect of gastro-intestinal nematode infection on sheep performance: a systematic review and meta-analysis. Parasit. Vectors 8, 1-11.
(18) Roeber, F., Jex, A. R., and Gasser, R. B. (2013) Impact of gastrointestinal parasitic nematodes of sheep, and the role of advanced molecular tools for exploring epidemiology and drug resistance—an Australian perspective. Parasit. Vectors 6, 1-13.
(19) Singh, S. K., Hodda, M., and Ash, G. J. (2013) Plant-parasitic nematodes of potential phytosanitary importance, their main hosts and reported yield losses. EPPO Bull. 43, 334-374.
(20) Sasser, J. N., and Freckman, D. W. (1987) A world perspective on nematology: the role of the society, in Vistas on nematology (Veech, J. A., and Dickson, D. W., Eds.), pp 7-14. Society of Nematology.
(21) McCarter, J. P. (2009) Molecular Approaches Toward Resistance to Plant-Parasitic Nematodes, in Cell Biology of Plant Nematode Parasitism (Berg, R. H., and Taylor, C. G., Eds.), pp 239-267. Springer-Verlag.
(22) Abd-Elgawad, M., and Askary, T. H. (2015) Impact of Phytonematodes on Agriculture Economy, in Biocontrol Agents of Phytonematodes (Askary, T. H., and Martinelli, P. R. P., Eds.), pp 3-49. CABI.
(23) Sasser, J. N. (1977) Worldwide Dissemination and Importance of the Root-knot Nematodes, Meloidogyne spp. J. Nematol. 9, 26-29.
(24) Abad, P., Gouzy, J., Aury, J. M., Castagnone-Sereno, P., Danchin, E. G. J., Deleury, E., Perfus-Barbeoch, L., Anthouard, V., Artiguenave, F., Blok, V. C., Caillaud, M. C., Coutinho, P. M., Dasilva, C., De Luca, F., Deau, F., Esquibet, M., Flutre, T., Goldstone, J. V., Hamamouch, N., Hewezi, T., Jaillon, O., Jubin, C., Leonetti, P., Magliano, M., Maier, T. R., Markov, G. V., McVeigh, P., Pesole, G., Poulain, J., Robinson-Rechavi, M., Sallet, E., Segurens, B., Steinbach, D., Tytgat, T., Ugarte, E., Van Ghelder, C., Veronico, P., Baum, T. J., Blaxter, M., Bleve-Zacheo, T., Davis, E. L., Ewbank, J. J., Favery, B., Grenier, E., Henrissat, B., Jones, J. T., Laudet, V., Maule, A. G., Quesneville, H., Rosso, M. N., Schiex, T., Smant, G., Weissenbach, J., and Wincker, P. (2008) Genome sequence of the metazoan plant-parasitic nematode Meloidogyne incognita. Nat. Biotechnol. 26, 909-915.
(25) Sutherland, I. A., and Leathwick, D. M. (2011) Anthelmintic resistance in nematode parasites of cattle: A global issue? Trends Parasitol. 27, 176-181.
(26) Sargison, N. (2011) Responsible use of anthelmintics for nematode control in sheep and cattle. In Pract. 33, 318-327.
(27) Jones, R. K. (2017) Nematode Control and Nematicides: Developments Since 1982 and Future Trends, in Nematology in South Africa: A View from the 21st Century (Fourie, H., Spaull, V., Jones, R., Daneel, M., and De Waele, D., Eds.), pp 129-150. Springer, Cham.
(28) Chitwood, D. J. (2003) Nematicides. United States Department of Agriculture
Agricultural Research Service.
(29) United Nations. (2002) Consolidated List of Products Whose Consumption and/or Sale Have Been Banned, Withdrawn, Severely Restricted or not Approved by Governments: Chemicals. Econ. Soc. Aff. New York.
(30) (2003) COUNCIL DECISION of 18 Mar. 2003 concerning the non-inclusion of aldicarb in Annex I to Council Directive 91/414/EEC and the withdrawal of authorisations for plant protection products containing this active substance. Off. J. Eur. Union.
(31) Zasada, I. A., Halbrendt, J. M., Kokalis-Burelle, N., Lamondia, J., Mckenry, M. V, and Noling, J. W. (2010) Managing Nematodes Without Methyl Bromide. Annu. Rev. Phytopathol. 25 48, 311-328.
(32) Lahm, G. P., Desaeger, J., Smith, B. K., Pahutski, T. F., Rivera, M. A., Meloro, T., Kucharczyk, R., Lett, R. M., Daly, A., Smith, B. T., Cordova, D., Thoden, T., and Wiles, J. A. (2017) The discovery of fluazaindolizine: A new product for the control of plant parasitic nematodes. Bioorg. Med. Chem. Lett. 27, 1572-1575.
(33) Kearn, J., Ludlow, E., Dillon, J., Connor, V. O., and Holden-Dye, L. (2014) Fluensulfone is a nematicide with a mode of action distinct from anticholinesterases and macrocyclic lactones. Pestic. Biochem. Physiol. 109, 44-57.
(34) Slomczynska, U., South, M. S., Bunkers, G. J., Edgecomb, D., Wyse-Pester, D., Selness, S., Ding, Y., Christiansen, J., Ediger, K., Miller, W., Charumilind, P., Hartmann, G., Williams, J., Dimmic, M., Shortt, B., Haakenson, W., Wideman, A., Crawford, M., Hresko, M., and Mccarter, J. (2015) Tioxazafen: A New Broad-Spectrum Seed Treatment Nematicide, in Discovery and Synthesis of Crop Protection Products (Maienfisch, P., and Stevenson, T. M., Eds.), pp 129-147. American Chemical Society.
(35) D′Errico, G., Giacometti, R., Roversi, P. F., D'Errico, F. P., and Woo, S. L. (2017) Mode of action and efficacy of iprodione against the root-knot nematode Meloidogyne incognita. Ann. Appl. Biol. 171, 506-510.
(36) Faske, T. R., and Hurd, K. (2015) Sensitivity of Meloidogyne incognita and Rotylenchulus reniformis to Fluopyram. J. Nematol. 47, 316-321.
(37) Monfort, W. S., Kirkpatrick, T. L., Long, D. L., and Rideout, S. (2006) Efficacy of a Novel Nematocidal Seed Treatment against Meloidogyne incognita on Cotton. J. Nematol. 38, 245-249.
(38) Kaplan, R. M. (2004) Drug resistance in nematodes of veterinary importance: a status report. Trends Parasitol. 20, 477-481.
(39) Bartley, D. J., Devin, L., Nath, M., and Morrison, A. A. (2015) Selection and characterisation of monepantel resistance in Teladorsagia circumcincta isolates. Int. J. Parasitol. Drugs Drug Resist. 5, 69-76.
(40) Sales, N., and Love, S. (2016) Resistance of Haemonchus sp. to monepantel and reduced efficacy of a derquantel/abamectin combination confirmed in sheep in NSW, Australia. Vet. Parasitol. 228, 193-196.
(41) Lewis, J. A., and Fleming, J. T. (1995) Basic Culture Methods, in Caenorhabditis elegans: Modern Biological Analysis of an Organism (Epstein, H. F., and Shakes, D. C., Eds.), pp 3-29. Academic Press, Inc.
(42) Burns, A. R., Luciani, G. M., Musso, G., Bagg, R., Yeo, M., Zhang, Y., Rajendran, L., Glavin, J., Hunter, R., Redman, E., Stasiuk, S., Schertzberg, M., Angus McQuibban, G., Caffrey, C. R., Cutler, S. R., Tyers, M., Giaever, G., Nislow, C., Fraser, A. G., MacRae, C. A., Gilleard, J., and Roy, P. J. (2015) Caenorhabditis elegans is a useful model for anthelmintic discovery. Nat. Commun. 6, 7485.
(43) Burns, A. R., Kwok, T. C. Y., Howard, A., Houston, E., Johanson, K., Chan, A., Cutler, S. R., McCourt, P., and Roy, P. J. (2006) High-throughput screening of small molecules for bioactivity and target identification in Caenorhabditis elegans. Nat. Protoc. 1, 1906-1914.
(44) Jindapunnapat, K., Reetz, N. D., Macdonald, M. H., Bhagavathy, G., and Meyer, S. L. 10 F. (2018) Activity of Vetiver Extracts and Essential Oil against Meloidogyne incognita. J. Nematol. 50, 147-162.
(45) Meyer, S. L. F., Chauhan, K. R., and MacDonald, M. H. (2016) EVALUATION OF ROSELLE (HIBISCUS SABDARIFFA) LEAF AND POMEGRANATE (PUNICA GRANATUM) FRUIT RIND FOR ACTIVITY AGAINST MELOIDOGYNE INCOGNITA. Nematropica 46, 85-96.
(46) Meyer, S. L. F., Zasada, I. A., Roberts, D. P., Vinyard, B. T., Lakshman, D. K., Lee, J.- K., Chitwood, D. J., and Carta, L. K. (2006) Plantago lanceolata and Plantago rugelii Extracts are Toxic to Meloidogyne incognita but not to Certain Microbes. J. Nematol. 38, 333-338.
(47) Wram, C. L. and Zasada, I. A. (2019) Short-Term Effects of Sublethal doses of Nematicides on Meloidogyne incognita. Phytopathology. 109(9), 1605-1613.
(48) Akhtar, M. and Malik, A. (2000) Roles of organic soil amendments and soil organisms in the biological control of plant-parasitic nematodes: a review. Bioresource Technology. 74(1), 35-47.
(49) Lacey L. A. and Georgis R. (2012) Entomopathogenic Nematodes for Control of Insect Pests Above and Below Ground with Comments on Commercial Production. Journal of Nematology. 44(2), 218-225.

1. A method for treating or preventing a nematode infection in a plant comprising administering to a plant in need thereof, an effective amount of one or more compounds of Formula (I) and/or salts and/or solvates thereof, 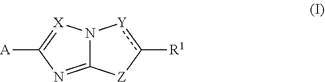
wherein: [symbol]
is a single bond or a double bond;
X is CR² or N;
Y is CR³ or N, when [symbol] is a double bond, or
Y is CHR³ or NR⁴ when [symbol] is a single bond;
Z is O or S;
A is a 6 or 10 membered aryl or a 5, 6, 8, 9 or 10 membered heteroaryl that is unsubstituted or substituted with one to five substituents independently selected from halo, cyano, NH₂, CO₂H, C(O)H, C₁₋₄alkyl, C(O)C₁₋₄alkyl, CO₂C₁₋₄alkyl, OC(O)C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), OC₁₋₄alkyleneOC₁₋₄alkyl, C₁₋₄alkyleneOC₁₋₄alkyl and OC₁₋₄alkyl;
R¹, R² and R³ are independently selected from H, halo, cyano, NH₂, CO₂H, C(O)H, C₁₋₄alkyl, C(O)C₁₋₄alkyl, CO₂C₁₋₄alkyl, NHC₁₋₄alkyl, N(C₁₋₄alkyl)(C₁₋₄alkyl), NHC(O)C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, OC₁₋₄alkyl, OC₁₋₄alkyleneOC₁₋₄alkyl, C₃₋₆cycloalkyl and C₃₋₆heterocycloalkyl;
R⁴ is selected from H, C₁₋₄alkyl, C(O)C₁₋₄alkyl, CO₂C₁₋₄alkyl, C(O)NH₂, C(O)NHC₁₋₄alkyl, C(O)N(C₁₋₄alkyl)(C₁₋₄alkyl), C₁₋₄alkyleneOC₁₋₄alkyl, C₃₋₆cycloalkyl and C₃₋₆heterocycloalkyl, and
each alkyl group in the compound of Formula I is optionally halo-substituted, provided that:
when A is unsubstituted phenyl or phenyl monosubstituted on the 4-position with halo, Z is S and Y is CR³, then at least one of R¹—R⁴ is other than H and R¹ and/or R3 are not halo, C₁₋₄alkyl, NH₂ or C(O)H; and
when A is pyridinyl or pyrimidinyl, Y is CR² and [symbol] is a double bond, then R¹ and R2 are each not C(O)NH₂, C(O)NHC₁₋₄alkyl or C(O)NHC₁₋₄fluoroalkyl.

2. The method of claim 1, wherein [symbol] is double bond.

3. The method of claim 1, wherein X is CR² or wherein X is O.

4. The method of claim 1, wherein Y is CR³¹ or wherein Y is N, or wherein Y is CHR³.

5. (canceled)

6. The method of claim 1, wherein Z is S.

7. (canceled)

8. (canceled)

9. The method of claim 1, wherein each alkyl group in the compound of Formula I is optionally fluoro-substituted.

10. The method of claim 1, wherein A is phenyl or naphthyl, each of which is unsubstituted or substituted.

11. The method of claim 1, wherein A is unsubstituted or substituted phenyl.

12. The method of claim 1, wherein A is substituted phenyl or substituted naphthyl.

13. The method of claim 1, wherein when A is a substituted 6 or 10 membered aryl, the substituents are independently selected from one to three of halo, cyano, NH₂, CO₂H, C(O)H, C₁₋₄alkyl, C(O)C₁₋₄ alkyl, CO₂C₁₋₄alkyl, OC(O)C₁₋₄alkyl, NHC₁₋₄alkyl, NHC(O)C₁₋₄alkyl, C(O)NH₂, C(O)NHC₁₋₄alkyl, OC₁₋₄alkyleneOC₁₋₄alkyl, C₁₋₄alkyleneOC₁₋₄alkyl and OC₁₋₄alkyl, and each alkyl group is optionally fluoro-substituted.

14. (canceled)

15. (canceled)

16. The method of claim 1, wherein when A is a 5, 6, 8, 9 or 10 membered heteroaryl that is unsubstituted or substituted.

17. (canceled)

18. (canceled)

19. (canceled)

20. (canceled)

21. (canceled)

22. (canceled)

23. The method claim 1, wherein X is CR² and Y is CR³ and R¹, R² and R³ are independently selected from H, Cl, Br, I, cyano, NH₂, CO₂H, C(O)H, CH₃, CF₃, CHF₂, OCH₃, OCF₃, OCHF₂, CH₂CH₃, OCH₂CH₃, CH(CH₃)₂, C(CH₃)₃, C(O)CH₃, C(O)CH₂CH₃, C(O)OCH₃, NHCH₃, NHC(O)CH₃, NHC(O)CH₂CH₃, C(O)NH₂, C(O)NHCH₃, CH₂OCH₃, OCH₂OCH₃, cyclopentyl, cyclohexyl, pyrolidin-1yl and piperidin-1-yl.

24. (canceled)

25. (canceled)

26. (canceled)

27. (canceled)

28. (canceled)

29. (canceled)

30. The method of claim 1, wherein Y is NR⁴ and R⁴ is selected from H, CH₃, CF₃, CHF₂, CH₂CH₃, CH(CH₃)₂, C(CH₃)₃, C(O)OCH₃, C(O)CH₃ and C(O)CH₂CH₃.

31. (canceled)

32. (canceled)

33. (canceled)

34. (canceled)

35. (canceled)

36. (canceled)

37. (canceled)

38. (canceled)

39. (canceled)

40. (canceled)

41. The method of claim 1, wherein the one or more compounds Formula (I) is selected from:

| Cpd | |
|---|---|
| No. | Structure |
| I-1 | 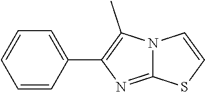 |
| I-2 | 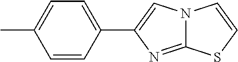 |
| I-3 | 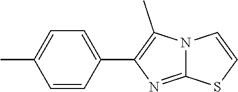 [SMILES: Cc1ccc(-c2nc3sccn3c2C)cc1] |
| I-4 | 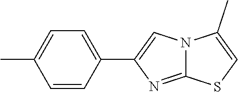 [SMILES: Cc1ccc(-c2cn3c(C)csc3n2)cc1] |
| I-5 | 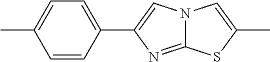 |
| I-6 | 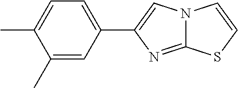 [SMILES: Cc1ccc(-c2cn3ccsc3n2)cc1C] |
| I-7 | 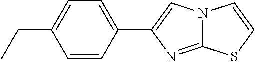 [SMILES: CCc1ccc(-c2cn3ccsc3n2)cc1] |
| I-8 | 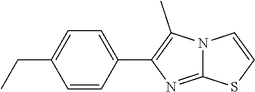 [SMILES: CCc1ccc(-c2nc3sccn3c2C)cc1] |
| I-9 | 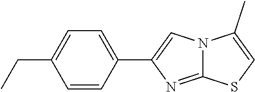 [SMILES: CCc1ccc(-c2cn3c(C)csc3n2)cc1] |
| I-10 | 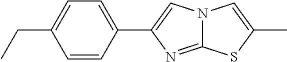 [SMILES: CCc1ccc(-c2cn3cc(C)sc3n2)cc1] |
| I-11 | 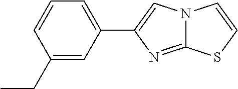 [SMILES: CCc1cccc(-c2cn3ccsc3n2)c1] |
| I-12 | 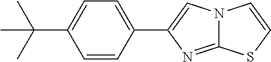 [SMILES: CC(C)(C)c1ccc(-c2cn3ccsc3n2)cc1] |
| I-13 | 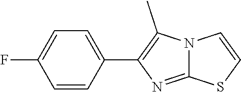 [SMILES: Cc1c(-c2ccc(F)cc2)nc2sccn12] |
| I-14 | 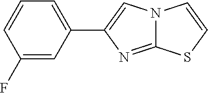 [SMILES: Fc1cccc(-c2cn3ccsc3n2)c1] |
| I-15 | 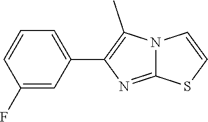 [SMILES: Cc1c(-c2cccc(F)c2)nc2sccn12] |
| I-16 | 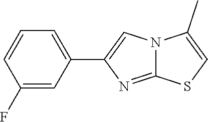 [SMILES: Cc1csc2nc(-c3cccc(F)c3)cn12] |
| I-17 | 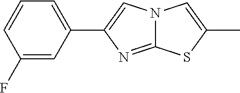 [SMILES: Cc1cn2cc(-c3cccc(F)c3)nc2s1] |
| I-18 | 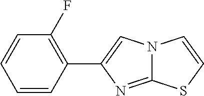 [SMILES: Fc1ccccc1-c1cn2ccsc2n1] |
| I-19 | 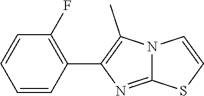 [SMILES: Cc1c(-c2ccccc2F)nc2sccn12] |
| I-20 | 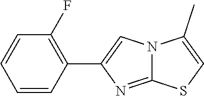 [SMILES: Cc1csc2nc(-c3ccccc3F)cn12] |
| I-21 | 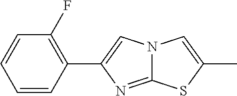 [SMILES: Cc1cn2cc(-c3ccccc3F)nc2s1] |
| I-22 | 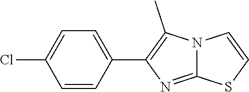 [SMILES: Cc1c(-c2ccc(Cl)cc2)nc2sccn12] |
| I-23 | 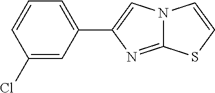 [SMILES: Clc1cccc(-c2cn3ccsc3n2)c1] |
| I-24 | 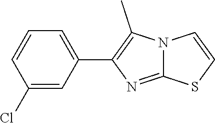 [SMILES: Cc1c(-c2cccc(Cl)c2)nc2sccn12] |
| I-25 | 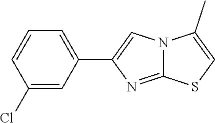 [SMILES: Cc1csc2nc(-c3cccc(Cl)c3)cn12] |
| I-26 | 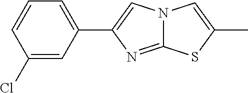 [SMILES: Cc1cn2cc(-c3cccc(Cl)c3)nc2s1] |
| I-27 | 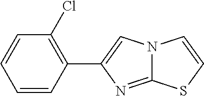 [SMILES: Clc1ccccc1-c1cn2ccsc2n1] |
| I-28 | 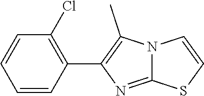 [SMILES: Cc1c(-c2ccccc2Cl)nc2sccn12] |
| I-29 | 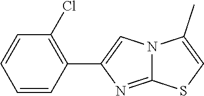 [SMILES: Cc1csc2nc(-c3ccccc3Cl)cn12] |
| I-30 | 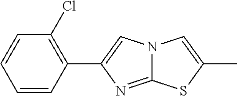 [SMILES: Cc1cn2cc(-c3ccccc3Cl)nc2s1] |
| I-31 | 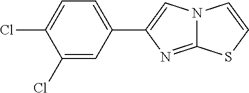 [SMILES: Clc1ccc(-c2cn3ccsc3n2)cc1Cl] |
| I-32 | 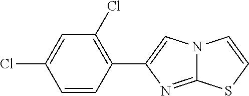 [SMILES: Clc1ccc(-c2cn3ccsc3n2)c(Cl)c1] |
| I-33 | 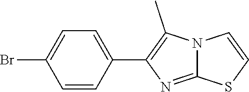 [SMILES: Cc1c(-c2ccc(Br)cc2)nc2sccn12] |
| I-34 | 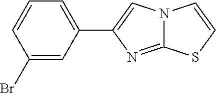 [SMILES: Brc1cccc(-c2cn3ccsc3n2)c1] |
| I-35 | 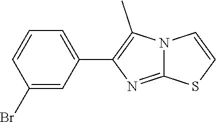 [SMILES: Cc1c(-c2cccc(Br)c2)nc2sccn12] |
| I-36 | 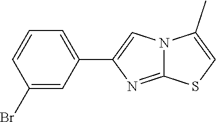 [SMILES: Cc1csc2nc(-c3cccc(Br)c3)cn12] |
| I-37 | 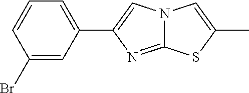 [SMILES: Cc1cn2cc(-c3cccc(Br)c3)nc2s1] |
| I-38 | 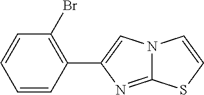 [SMILES: Brc1ccccc1-c1cn2ccsc2n1] |
| I-39 | 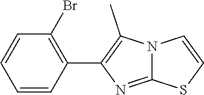 [SMILES: Cc1c(-c2ccccc2Br)nc2sccn12] |
| I-40 | 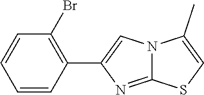 [SMILES: Cc1csc2nc(-c3ccccc3Br)cn12] |
| I-41 | 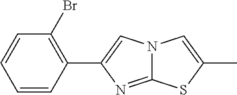 [SMILES: Cc1cn2cc(-c3ccccc3Br)nc2s1] |
| I-42 | 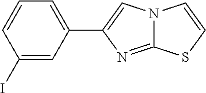 [SMILES: Ic1cccc(-c2cn3ccsc3n2)c1] |
| I-43 | 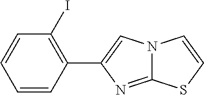 [SMILES: Ic1ccccc1-c1cn2ccsc2n1] |
| I-44 | 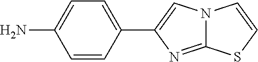 [SMILES: Nc1ccc(-c2cn3ccsc3n2)cc1] |
| I-45 | 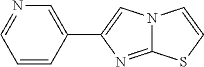 [SMILES: c1cncc(-c2cn3ccsc3n2)c1] |
| I-46 | 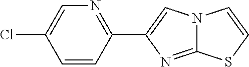 [SMILES: Clc1ccc(-c2cn3ccsc3n2)nc1] |
| I-47 | 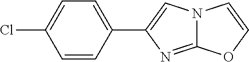 [SMILES: Clc1ccc(-c2cn3ccoc3n2)cc1] |
| I-48 | 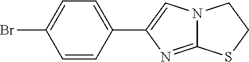 [SMILES: Brc1ccc(-c2cn3c(n2)SCC3)cc1] |
| I-49 | 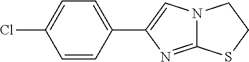 [SMILES: Clc1ccc(-c2cn3c(n2)SCC3)cc1] |
| I-50 | 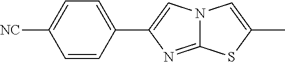 [SMILES: Cc1cn2cc(-c3ccc(C#N)cc3)nc2s1] |
| I-51 | 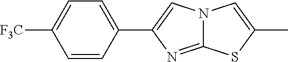 [SMILES: Cc1cn2cc(-c3ccc(C(F)(F)F)cc3)nc2s1] |
| I-52 | 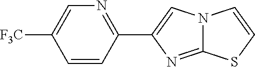 [SMILES: FC(F)(F)c1ccc(-c2cn3ccsc3n2)nc1] |
| I-53 | 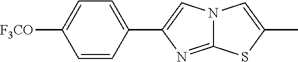 [SMILES: Cc1cn2cc(-c3ccc(OC(F)(F)F)cc3)nc2s1] |
| I-54 | 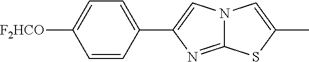 [SMILES: Cc1cn2cc(-c3ccc(OC(F)F)cc3)nc2s1] |
| I-55 | 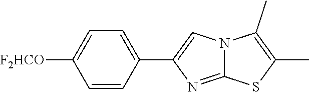 [SMILES: Cc1sc2nc(-c3ccc(OC(F)F)cc3)cn2c1C] |
| I-56 | 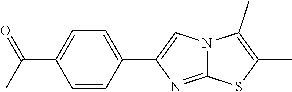 [SMILES: CC(=O)c1ccc(-c2cn3c(C)c(C)sc3n2)cc1] |
| I-57 | 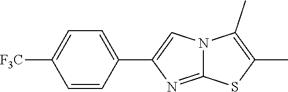 [SMILES: Cc1sc2nc(-c3ccc(C(F)(F)F)cc3)cn2c1C] |
| I-58 | 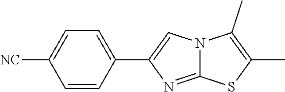 [SMILES: Cc1sc2nc(-c3ccc(C#N)cc3)cn2c1C] |
| I-59 | 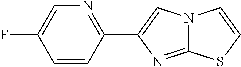 [SMILES: Fc1ccc(-c2cn3ccsc3n2)nc1] |
| I-60 | 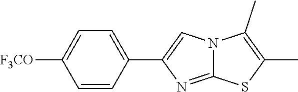 [SMILES: Cc1sc2nc(-c3ccc(OC(F)(F)F)cc3)cn2c1C] |
| I-61 | 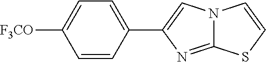 [SMILES: FC(F)(F)Oc1ccc(-c2cn3ccsc3n2)cc1] |
| I-62 | 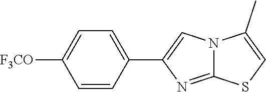 [SMILES: Cc1csc2nc(-c3ccc(OC(F)(F)F)cc3)cn12] |
| I-63 | 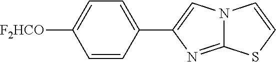 [SMILES: FC(F)Oc1ccc(-c2cn3ccsc3n2)cc1] |
| I-64 | 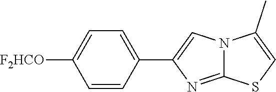 [SMILES: Cc1csc2nc(-c3ccc(OC(F)F)cc3)cn12] |
| I-65 | 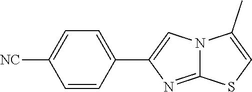 [SMILES: Cc1csc2nc(-c3ccc(C#N)cc3)cn12] |
| I-66 | 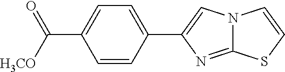 [SMILES: COC(=O)c1ccc(-c2cn3ccsc3n2)cc1] |
| I-67 | 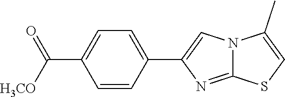 [SMILES: COC(=O)c1ccc(-c2cn3c(C)csc3n2)cc1] |
| I-68 | 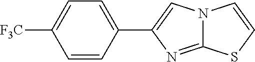 [SMILES: FC(F)(F)c1ccc(-c2cn3ccsc3n2)cc1] |
| I-69 | 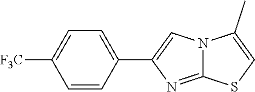 [SMILES: Cc1csc2nc(-c3ccc(C(F)(F)F)cc3)cn12] |
| I-70 | 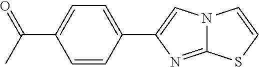 [SMILES: CC(=O)c1ccc(-c2cn3ccsc3n2)cc1] |
| I-71 | 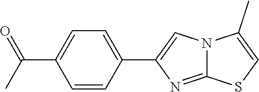 [SMILES: CC(=O)c1ccc(-c2cn3c(C)csc3n2)cc1] |
| I-72 | 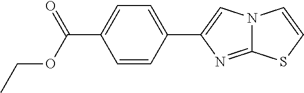 [SMILES: CCOC(=O)c1ccc(-c2cn3ccsc3n2)cc1] |
| I-73 | 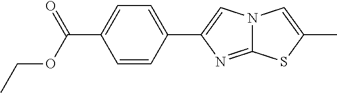 [SMILES: CCOC(=O)c1ccc(-c2cn3cc(C)sc3n2)cc1] |
| I-74 | 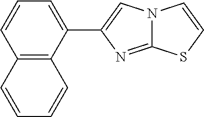 [SMILES: c1ccc2c(-c3cn4ccsc4n3)cccc2c1] |
| I-75 | 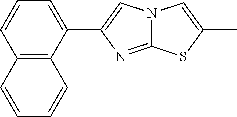 [SMILES: Cc1cn2cc(-c3cccc4ccccc34)nc2s1] |
| I-76 | 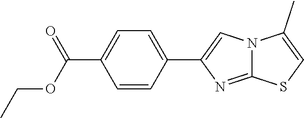 [SMILES: CCOC(=O)c1ccc(-c2cn3c(C)csc3n2)cc1] |
| I-77 | 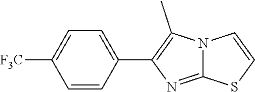 [SMILES: Cc1c(-c2ccc(C(F)(F)F)cc2)nc2sccn12] |
| I-78 | 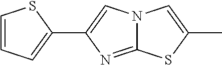 |
| I-79 | 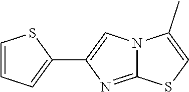 |
| I-80 | 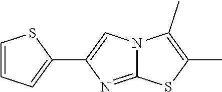 |
| I-81 | 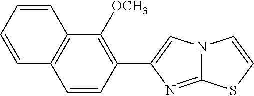 [SMILES: COc1c(-c2cn3ccsc3n2)ccc2ccccc12] |
| I-82 | 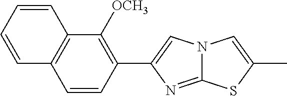 [SMILES: COc1c(-c2cn3cc(C)sc3n2)ccc2ccccc12] |
| I-83 | 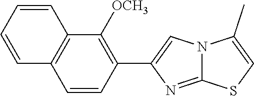 [SMILES: COc1c(-c2cn3c(C)csc3n2)ccc2ccccc12] |
| I-84 | 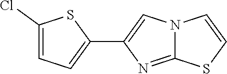 [SMILES: Clc1ccc(-c2cn3ccsc3n2)s1] |
| I-85 | 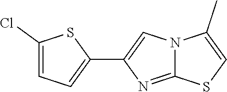 |
| I-86 | 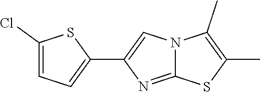 [SMILES: Cc1sc2nc(-c3ccc(Cl)s3)cn2c1C] |
| I-87 | 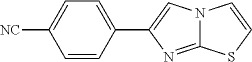 [SMILES: N#Cc1ccc(-c2cn3ccsc3n2)cc1] |
| I-88 | 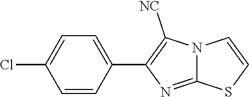 [SMILES: N#Cc1c(-c2ccc(Cl)cc2)nc2sccn12] |
| I-89 | 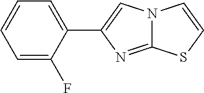 [SMILES: Fc1ccccc1-c1cn2ccsc2n1] |
| I-90 | 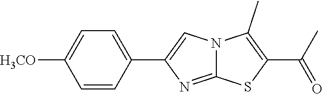 [SMILES: COc1ccc(-c2cn3c(C)c(C(C)=O)sc3n2)cc1] |
| I-91 | 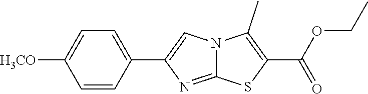 [SMILES: CCOC(=O)c1sc2nc(-c3ccc(OC)cc3)cn2c1C] |
| I-92 | 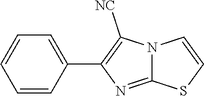 [SMILES: N#Cc1c(-c2ccccc2)nc2sccn12] |
| I-93 | 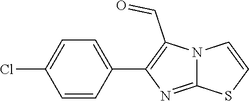 [SMILES: O=Cc1c(-c2ccc(Cl)cc2)nc2sccn12] |
| I-94 | 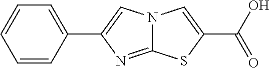 [SMILES: O=C(O)c1cn2cc(-c3ccccc3)nc2s1] |
| I-95 | 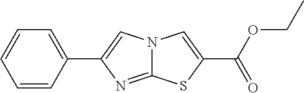 [SMILES: CCOC(=O)c1cn2cc(-c3ccccc3)nc2s1] |
| I-96 | 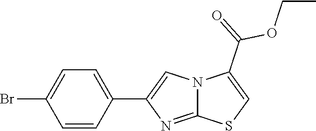 [SMILES: CCOC(=O)c1csc2nc(-c3ccc(Br)cc3)cn12] |
| I-97 | 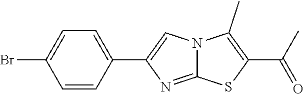 [SMILES: CC(=O)c1sc2nc(-c3ccc(Br)cc3)cn2c1C] |
| I-98 | 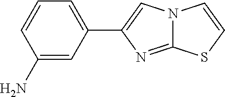 [SMILES: Nc1cccc(-c2cn3ccsc3n2)c1] |
| I-99 | 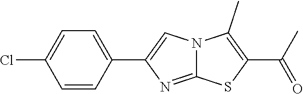 [SMILES: CC(=O)c1sc2nc(-c3ccc(Cl)cc3)cn2c1C] |
| I-100 | 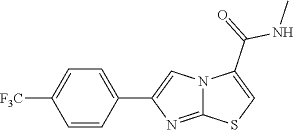 [SMILES: CNC(=O)c1csc2nc(-c3ccc(C(F)(F)F)cc3)cn12] |
| I-101 | 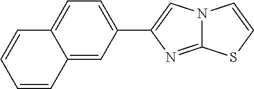 [SMILES: c1ccc2cc(-c3cn4ccsc4n3)ccc2c1] |
| I-102 | 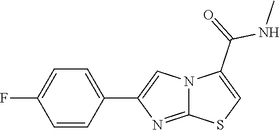 [SMILES: CNC(=O)c1csc2nc(-c3ccc(F)cc3)cn12] |
| I-103 | 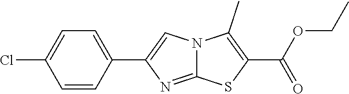 [SMILES: CCOC(=O)c1sc2nc(-c3ccc(Cl)cc3)cn2c1C] |
| I-104 | 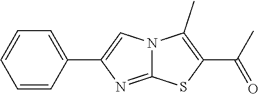 [SMILES: CC(=O)c1sc2nc(-c3ccccc3)cn2c1C] |
| I-105 | 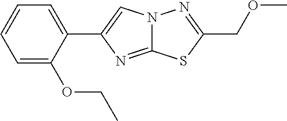 [SMILES: CCOc1ccccc1-c1cn2nc(COC)sc2n1] |
| I-106 | 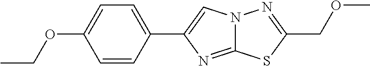 [SMILES: CCOc1ccc(-c2cn3nc(COC)sc3n2)cc1] |
| I-107 | 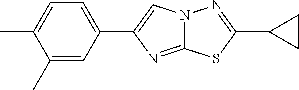 [SMILES: Cc1ccc(-c2cn3nc(C4CC4)sc3n2)cc1C] |
| I-108 | 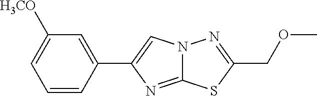 [SMILES: COCc1nn2cc(-c3cccc(OC)c3)nc2s1] |
| I-109 | 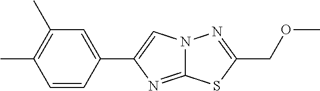 |
| I-110 | 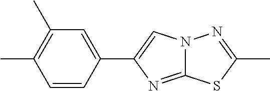 |
| I-111 | 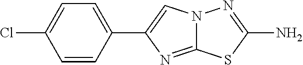 [SMILES: Nc1nn2cc(-c3ccc(Cl)cc3)nc2s1] |
| I-112 | 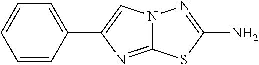 [SMILES: Nc1nn2cc(-c3ccccc3)nc2s1] |
| I-113 | 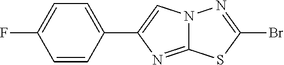 [SMILES: Fc1ccc(-c2cn3nc(Br)sc3n2)cc1] |
| I-114 | 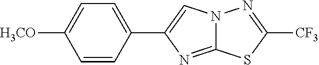 [SMILES: COc1ccc(-c2cn3nc(C(F)(F)F)sc3n2)cc1] |
| I-115 | 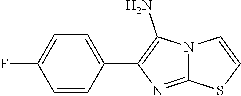 [SMILES: Nc1c(-c2ccc(F)cc2)nc2sccn12] |
| I-116 | 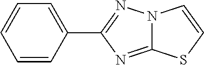 [SMILES: c1ccc(-c2nc3sccn3n2)cc1] |
| I-117 | 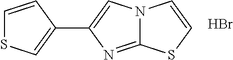 [SMILES: c1cc(-c2cn3ccsc3n2)cs1 ; Br] |
| I-118 | 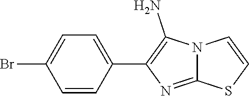 [SMILES: Nc1c(-c2ccc(Br)cc2)nc2sccn12] |
| I-119 | 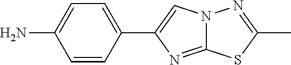 [SMILES: Cc1nn2cc(-c3ccc(N)cc3)nc2s1] |
| I-120 | 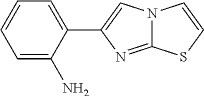 [SMILES: Nc1ccccc1-c1cn2ccsc2n1] |
| I-121 | 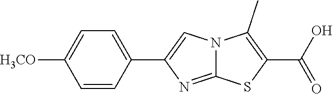 [SMILES: COc1ccc(-c2cn3c(C)c(C(=O)O)sc3n2)cc1] |
| I-122 | 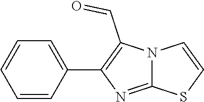 [SMILES: O=Cc1c(-c2ccccc2)nc2sccn12] |
| I-123 | 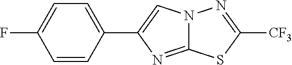 [SMILES: Fc1ccc(-c2cn3nc(C(F)(F)F)sc3n2)cc1] |
| I-124 | 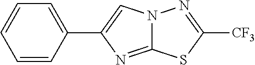 [SMILES: FC(F)(F)c1nn2cc(-c3ccccc3)nc2s1] |
| I-125 | 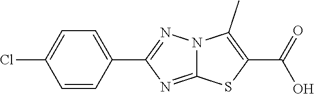 [SMILES: Cc1c(C(=O)O)sc2nc(-c3ccc(Cl)cc3)nn12] |
| I-126 | 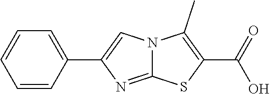 [SMILES: Cc1c(C(=O)O)sc2nc(-c3ccccc3)cn12] |
| I-127 | 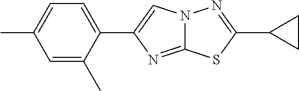 [SMILES: Cc1ccc(-c2cn3nc(C4CC4)sc3n2)c(C)c1] |
| I-128 | 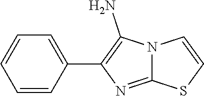 [SMILES: Nc1c(-c2ccccc2)nc2sccn12] |
| I-129 | 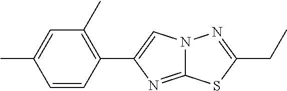 [SMILES: CCc1nn2cc(-c3ccc(C)cc3C)nc2s1] |
| I-130 | 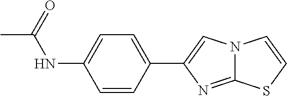 [SMILES: CC(=O)Nc1ccc(-c2cn3ccsc3n2)cc1] |
| I-131 | 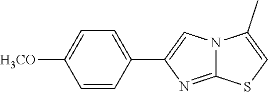 [SMILES: COc1ccc(-c2cn3c(C)csc3n2)cc1] |
| I-132 | 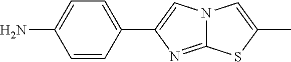 [SMILES: Cc1cn2cc(-c3ccc(N)cc3)nc2s1] |
| I-133 | 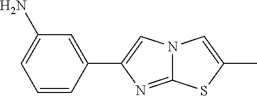 [SMILES: Cc1cn2cc(-c3cccc(N)c3)nc2s1] |
| I-134 | 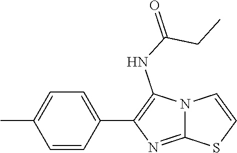 [SMILES: CCC(=O)Nc1c(-c2ccc(C)cc2)nc2sccn12] |
| I-135 | 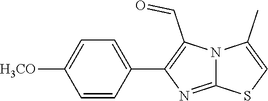 [SMILES: COc1ccc(-c2nc3scc(C)n3c2C=O)cc1] |
| I-136 | 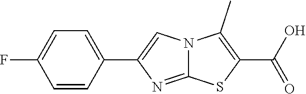 [SMILES: Cc1c(C(=O)O)sc2nc(-c3ccc(F)cc3)cn12] |
| I-137 | 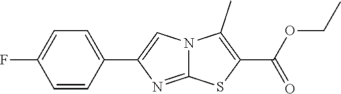 [SMILES: CCOC(=O)c1sc2nc(-c3ccc(F)cc3)cn2c1C] |
| I-138 | 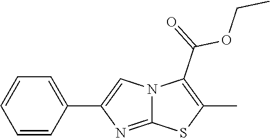 [SMILES: CCOC(=O)c1c(C)sc2nc(-c3ccccc3)cn12] |
| I-139 | 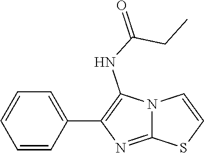 [SMILES: CCC(=O)Nc1c(-c2ccccc2)nc2sccn12] |
| I-140 | 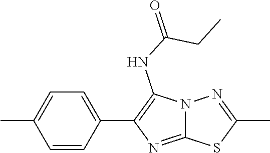 |
| I-141 | 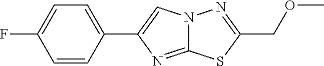 [SMILES: COCc1nn2cc(-c3ccc(F)cc3)nc2s1] |
| I-142 | 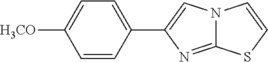 [SMILES: COc1ccc(-c2cn3ccsc3n2)cc1] |
| I-143 | 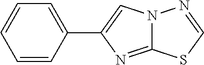 [SMILES: c1ccc(-c2cn3ncsc3n2)cc1] |
| I-144 | 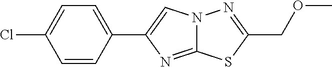 [SMILES: COCc1nn2cc(-c3ccc(Cl)cc3)nc2s1] |
| I-145 | 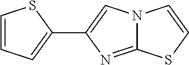 [SMILES: c1csc(-c2cn3ccsc3n2)c1] |
| I-146 | 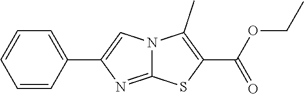 [SMILES: CCOC(=O)c1sc2nc(-c3ccccc3)cn2c1C] |
| I-147 | 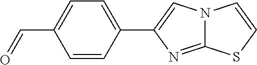 [SMILES: O=Cc1ccc(-c2cn3ccsc3n2)cc1] |
| I-148 | 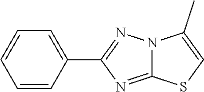 |
| I-149 | 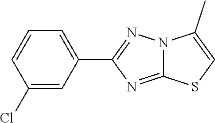 [SMILES: Cc1csc2nc(-c3cccc(Cl)c3)nn12] |
| I-150 | 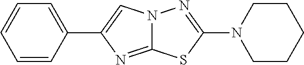 [SMILES: c1ccc(-c2cn3nc(N4CCCCC4)sc3n2)cc1] |
| I-151 | 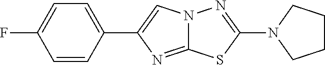 [SMILES: Fc1ccc(-c2cn3nc(N4CCCC4)sc3n2)cc1] |
| I-152 | 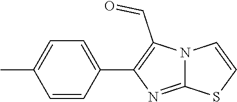 [SMILES: Cc1ccc(-c2nc3sccn3c2C=O)cc1] |
| I-153 | 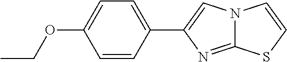 [SMILES: CCOc1ccc(-c2cn3ccsc3n2)cc1] |
| I-154 | 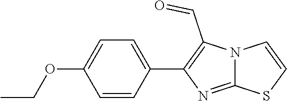 [SMILES: CCOc1ccc(-c2nc3sccn3c2C=O)cc1] |
| I-155 | 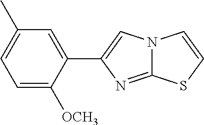 [SMILES: COc1ccc(C)cc1-c1cn2ccsc2n1] |
| I-156 | 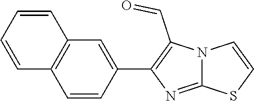 [SMILES: O=Cc1c(-c2ccc3ccccc3c2)nc2sccn12] |
| I-157 | 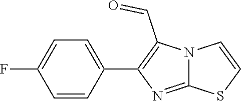 [SMILES: O=Cc1c(-c2ccc(F)cc2)nc2sccn12] |
| I-158 | 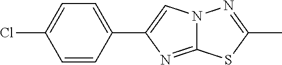 [SMILES: Cc1nn2cc(-c3ccc(Cl)cc3)nc2s1] |
| I-159 | 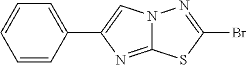 [SMILES: Brc1nn2cc(-c3ccccc3)nc2s1] |
| I-160 | 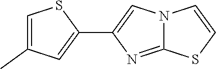 |
| I-161 | 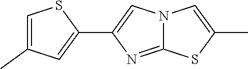 |
| I-162 | 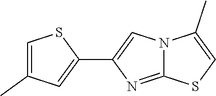 |
| | and |
| I-163 | 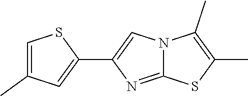 |

and/or salts and/or solvates thereof.

42. The method of claim 1, wherein the nematode infection is an infection of a nematode selected from the following genera: Meloidogyne, Heterodera, Globodera, Pratylenchus, Rotylenchulus, Hoplolaimus, Bolonolaimus, Longidorus, Paratrichodorus, Ditylenchus, Bursaphalencus, Xiphinema, Nacobbus, Aphelenchoides, Helicotylenchus, Radopholus, Hirschmanniella, Tylenchorhynchus, Trichodorus, Anguina, Criconema, Criconemella, Criconemoides, Mesocriconema, Dolichodorus, Hemicycliophora, Hemicriconemoides, Scutellonema, Tylenchulus, Subanguina, Hypsoperine, Macroposthonia, Melinius, Punctodera, and Quinisulcius.

43. (canceled)

44. (canceled)

45. The method of claim 1, wherein the plant is an agricultural crop or a cultivated plant.

46. The method of claim 1, wherein the plant is selected from one or more of soybeans, cotton, flax, hemp, jute, corn, tobacco, nuts, almonds, coffee, tea, pepper, grapevines, hops, wheat, barley, rye, oats, rice, maize, sorghum, apples, pears, plums, peaches, banana, plantains, cherries, strawberries, raspberries, blackberries, beans, lentils, peas, soya, oilseed rape, mustard, poppies, olives, sunflowers, coconut, castor, cocoa, ground nuts, spinach, asparagus, lettuce, cabbages, carrots, onions, tomatoes, potatoes, bell peppers, cucumbers, melons, pumpkins, sugar cane, sugar beet, fodder beet, avocado, cinnamon, camphor, oranges, tangerines, lemons, limes, grapefruit, latex plants, ornamental plants and turf grasses.

47. The method of claim 1, wherein the treatment further comprises treating a disease, disorder or condition arising from the nematode infection in the plant.

48. (canceled)

49. The method of claim 1, wherein the method comprises applying to the plant, to the soil surrounding the plant, and/or to the seeds of the plant an effective amount of the one or more compounds of Formula (I) and/or salts and/or solvates thereof.

50. The method of claim 1, wherein the one or more compounds of Formula (I) and/or salts and/or solvates thereof is used in combination with other known agents useful for treating or preventing a nematode infection.

51. A composition for treating or preventing a nematode infection or a disease, a disorder, or a condition arising from a nematode infection in a plant comprising an effective amount of one or more compounds of Formula (I), and/or salts and/or solvates thereof as defined in claim 1.

52-74. (canceled)
